(19) United States
(12) Patent Application Publication
(10) Pub. No.: US 2006/0019971 A1
(43) Pub. Date: Jan. 26, 2006

(54) Treatment of cardiovascular disease with inhibitors of p38 kinase

(76) Inventors: Linda S. Higgins, Palo Alto, CA (US); Moon Lim, Redwood City, CA (US)

Correspondence Address: Samuel M. Kais;Scios Inc., 6500 Paseo Padre Parkway, Fremont, CA, 94555, US

(21) Appl. No.: 10/956,402

(22) Filed: Sep. 30, 2004

Related U.S. Application Data
(60) Provisional application No. 60/507,768, filed on Sep. 30, 2003.

(51) Int. Cl. A61K 31/519

(52) U.S. Cl. 514/262.1

(57) ABSTRACT

The present invention concerns methods of treatment for acute coronary syndrome using a pharmaceutically effective amount of an inhibitor of p38 MAP kinase. More specifically, the invention concerns the treatment of cardiovascular disorders associated with atherosclerosis.

### CROSS-REFERENCE TO RELATED APPLICATIONS

This application claims priority from U.S. patent application Ser. No. 60/507,768 filed 30 Sep. 2003. The contents of that document are incorporated herein for reference.

### BACKGROUND OF THE INVENTION

1. Field of the Invention

The present invention concerns methods of treatment for acute coronary syndrome using a pharmaceutically effective amount of p38 MAP kinase. More specifically, the invention concerns the treatment of cardiovascular disease including atherosclerosis.

2. Background

C-reactive protein (CRP) is a substance produced in the liver when arteries become inflamed. Arterial inflammation plays a central role in atherosclerosis and its complications. See Libby P., Nature. 2002;420:868-874; Libby P, Aikawa M, Nat Med. 2002 ;8:1257-1262; Libby, Ridker, Maseri, Circulation. 2002;105:1135-1143. Review; Bhatt D L, Topol E J, Circulation. 2002;106:136-140.. It is involved in leukocyte recruitment, pro-inflammmatory cytokine expression and thrombosis that is responsible for myocardial infarction and strokes. CRP has long been considered as a marker of inflammation but recently emerged as one of the strongest prognostic markers of cardiovascular events such as atherosclerosis, myocardial infarction, stroke, and vascular death in a variety of settings. See Blake G J, Ridker P M, J Intern Med. 2002;252:283-294; Blake G J, Ridker P M, Arterioscler Thromb Vasc Biol. 2002;22:1512-1513; Saadeddin S M, Habbab M A, Ferns G A, Med Sci Monit. 2002;8:RA5-12; Benzaquen L R, Yu H, Rifai N. Crit Rev Clin Lab Sci. 2002;39:459-497. Elevated CRP levels predict poor prognosis of future cardiac events both in patients with coronary disease (Blake G J, Ridker P M, Circ Res. 2001; 89: 763-771) and in apparently healthy men and women (Benzaquen L R, Yu H, Rifai N, Crit Rev Clin Lab Sci. 2002;39:459-497). Given the association of arterial inflammation in coronary disease, particularly in patients presenting with high levels of CRP, anti-inflammatory therapy appears to have a therapeutic benefit. Conventional therapies for alleviating the inflammatory response include aspirin (Ridker P M, Cushman M, Stampfer M J, Tracy R P, Hennekens C H, N Engl J Med. 1997;336:973-979) and statins. Many of the patients admitted to cardiac units are under such therapy. However, nearly 75% of such patients revisit the cardiac units. Although the conventional therapies provide temporary benefit, they do not target the specific p38 mediated mechanisms which are implicated in “unstable” atheromatous plaques and acute coronary disease.

### SUMMARY OF THE INVENTION

The present invention is directed to mechanisms by which CRP and its negative effects are mediated through the p38 MAP kinase pathway. In one embodiment, the invention is directed to a method of treating various disorders associated with the presence and/or enhanced activity of CRP wherein said method comprises the administration of a pharmaceutically effective amount of p38 MAP kinase inhibitor to a patient in need thereof.

In a preferred embodiment, the invention is directed to a method of treating cardiovascular disease wherein said method comprises the administration of a pharmaceutically effective amount of an inhibitor of p38 MAP kinase to a patient in need thereof.

In another preferred embodiment, the invention is directed to a method of treating acute coronary disease wherein said method comprises the administration of a pharmaceutically effective amount of an inhibitor of p38 MAP kinase to a patient in need thereof.

In another preferred embodiment, the invention is directed to a method of treating atherosclerosis wherein said method comprises the administration of a pharmaceutically effective amount of an inhibitor of p38 MAP kinase to a patient in need thereof.

In yet another embodiment, the invention is directed to a method of treating arterial inflammation wherein said method comprises the administration of a pharmaceutically effective amount of an inhibitor of p38 MAP kinase to a patient in need thereof.

### BRIEF DESCRIPTION OF THE FIGURES

FIGS. 1a and 1b are respectively a Western blot and a graph summarizing the results of such blot. The effect of CRP on p38 MAP kinase activation in HPBMCs is illustrated in these figures. Also illustrated is the effect of a p38 inhibitor on p38 kinase activation by CRP. 1.5×10⁶ HPBMCs were plated in 48-well. After 1 hour the medium was removed and cells were incubated with or without 10 μg/ml of purified human CRP in serum-free RPMI 1640. In parallel experiments, cells were treated with 10% FCS together with the CRP treatment. To demonstrate p38 antagonism, the p38 inhibitor was added to a final concentration of 1 μM in the parallel treatment. After 5 minutes cells were lysed and phspho-p38 level was determined by Western. Phospho-p38 signal was normalized by GAPDH signal.

FIGS. 2a-c are graphs which summarize data that demonstrates CRP promotion of cytokine IL-6 secretion via p38 signaling. FIG. 2(a) illustrates both the effect of CRP on IL-6 secretion from HPBMCs and its modulation by a p38 inhibitor. 8×10⁵ HPBMCs were plated in 96-well. After 1 hour medium was removed and cells were incubated with or without 10 μg/ml of purified human CRP in serum-free RPMI 1640. In parallel experiments cells were treated with 10% FCS together with the CRP treatment. For the p38 antagonism, the p38 inhibitor was added to a final concentration of 1 μM in the parallel treatment. After 6 hours the conditioned medium was collected and IL-6 and IL-8 levels were determined by ELISA. FIG. 2(b) illustrates the effects of p38 inhibitor concentration on CRP induced IL-6 secretion from HPBMCs. 3×10⁵ HPBMCs were plated in 96-well. After 1 hour medium was removed and cells were incubated with 10 μg/ml of purified human CRP plus 10% FCS in the presence of varying concentrations of p38 inhibitor. After 4 hours conditioned medium were collected and IL-6 level was determined by ELISA. FIG. 2(c) illustrates both the effect of CRP on IL-6 gene expressions in HPBMCs and its modulation by p38 inhibitor. 5×10⁶ HPBMCs were plated in 6-well. After 1 hour medium was removed and cells were treated with vehicle or 1 μM p38 inhibitor in RPMI 1640 medium. After 1 hour cells were stimulated with 10% FCS only or 10 μg/ml CRP plus 10% FCS. After 6 hours RNA was extracted from the cells and IL-6 expression level was determined by real-time PCR as described in Experimental section.

FIG. 3a-b are graphs which summarize data that demonstrate CRP promotes chemokine IL-8 secretion via p38 signaling. FIG. 3(a) demonstrates the effect of CRP on IL-8 secretion from HPBMCs and its modulation by p38 inhibitor. 8×10⁵ HPBMCs were plated in 96-well. After 1 hour medium was removed and cells were incubated with or without 10 μg/ml of purified human CRP in serum-free RPMI 1640. In parallel experiments cells were treated with 10% FCS together with the CRP treatment. For the p38 antagonism, p38 inhibitor was added to a final concentration of 1 μM in the parallel treatment. After 6 hours conditioned medium were collected and IL-6 and IL-8 levels were determined by ELISA. FIG. 3(b) demonstrates the effect of CRP on IL-8 gene expression in HPBMCs and its modulation by p38 inhibitor. 5×10⁶ HPBMCs were plated in 6-well. After 1 hour medium was removed and cells were treated with vehicle or 1 μM p38 inhibitor in RPMI 1640 medium. After 1 hour cells were stimulated with 10% FCS only or 10 μg/ml CRP plus 10% FCS. After 2 hours RNA was extracted from the cells and IL-8 expression level was determined by real-time PCR as described in the Experimental section.

FIGS. 4a-d. are graphs which summarize data that demonstrates CRP induces TNFα and IL-1β secretion via the p38 pathway. FIGS. 4(a) and (b) demonstrate the effects of CRP on IL-1b (a) and TNFa (b) secretion from HPBMCs and their modulation by p38 inhibitor. 5×10⁶ HPBMCs were plated in 6-well. After 1 hour medium was removed and cells were treated with vehicle or 1μM p38 inhibitor in RPMI 1640 medium. After 1 hour cells were stimulated with 10% FCS only or 10 μg/ml CRP plus 10% FCS. After 6 hours conditioned medium was collected and IL-1b and TNFa levels were determined by ELISA. FIGS. 4(c) and (d) demonstrate the effects of CRP on cytokine gene expressions in HPBMCs and their modulation by p38 inhibitor. 5×10⁶ HPBMCs were plated in 6-well. After I hour medium was removed and cells were treated with vehicle or 1M p38 inhibitor in RPMI 1640 medium. After 1 hour cells were stimulated with 10% FCS only or 10 μg/ml CRP plus 10% FCS. After 2 hours RNA was extracted from the cells and TNFa (c) and IL-1b (d) expression levels were determined by real-time PCR as described in the Experimental section.

FIG. 5. is a graph which summarizes data that demonstrates the effect of CRP on COX-2 gene expression in HPBMCs and its modulation by p38 inhibitor. 5×10⁶ HPBMCs were plated in 6-well. After 1 hour medium was removed and cells were treated with vehicle or 1 μM p38 inhibitor in RPMI 1640 medium. After 1 hour cells were stimulated with 10% FCS only or 10 μg/ml CRP plus 10% FCS. After 6 hours RNA was extracted from the cells and COX-2 gene expression level was determined by real-time PCR as described in the Experimental section.

FIG. 6. is a graph which summarizes data that demonstrates the effect of CRP on ET-1 gene expression in HPBMCs and its inhibition by p38 inhibitor. 5×10⁶ HPBMCs were plated in 6-well. After 1 hour medium was removed and cells were treated with vehicle or 1 μM P38 INHIBITOR in RPMI 1640 medium. After 1 hour cells were stimulated with 10% FCS only or 10 μg/ml CRP plus 10% FCS. After 6 hours RNA was extracted from the cells and ET-1 gene expression level was determined by real-time PCR as described in the Experimental section.

FIG. 7(a-c) are a Western blot and graphs summarizing results which demonstrate that CRP augments prothrombotic Tissue Factor expression via p38 signaling. The figures illustrate the effects of CRP on TF protein and mRNA expression in HPBMCs and their modulation by p38 inhibitor. 1.5×10⁶ HPBMCs were plated in 48-well. After 1 hour medium was removed and cells were incubated with or without 10 μg/ml of purified human CRP in serum-free RPMI 1640. In parallel experiments, cells were treated with 10% FCS together with the CRP treatment. For the p38 antagonism, p38 inhibitor was added to a final concentration of 1 μM in the parallel treatment. After 16 hours cells were lysed and TF protein level was determined by Western blot. The results are shown in FIGS. 7(a) and 7(b). TF signal was normalized by GAPDH signal. As shown in FIG. 7(c), after 6 hours TF mRNA level were determined by real-time PCR as described in the Experimental section.

### DESCRIPTION OF THE INVENTION

Definitions:

A “pharmaceutically effective amount” is intended an amount of a compound that, when administered to a mammal for treating a condition, disorder or disease, is sufficient to elicit a cellular response that is clinically significant, without excessive levels of side effects.

“Mammal” refers to any animal classified as a mammal, including humans, domestic and farm animals, and zoo, sports, and pet companion animals such as a household pet and other domesticated animal such as, but not limited to, cattle, sheep, ferrets, swine, horses, poultry, rabbits, goats, dogs, cats and the like. Preferred companion animals are dogs and cats. Preferably, the mammal is human.

A “patient” is intended a mammal, preferably a human, in need of treatment of a condition, disorder or disease.

Description

Atherosclerosis is clinically recognized under two pathophysiologically distinct syndromes: stable coronary syndromes resulting from severe stenoses and unstable acute coronary syndromes resulting from different vascular inflammation frequently superimposed with thrombosis, which results in serious cardiac events. In the inflammation theory, the entry of inflammatory cells such as monocytes into the arterial wall plays a pivotal role in atherosclerosis. See for example Saadeddin S M, Habbab M A, Ferns G A, Med Sci Monit. 2002;8:RA5-12; and Benzaquen L R, Yu H, Rifai N., Crit Rev Clin Lab Sci. 2002;39:459-497. During inflammation, the endothelial cell lining on the arterial wall is activated and expresses adhesion molecules and chemokines. This facilitates the recruitment of circulating monocytes and other leukocytes. Once the recruited monocytes emigrate into vessel wall they become tissue macrophages, which can uptake lipid droplets to form foam cells. Continuous entry of these monocytes into the arterial wall promotes the lesion development. In the plaque, foam cells produce inflammatory mediators and growth factors, which can damage tissue and promote further inflammation and stenosis. In unstable acute coronary syndromes thin fibrous cap surrounding the lesion is prone to rupture by proteases produced by inflammatory leukocytes in the plaque or by vasoconstriction. This can expose activated macrophages and endothelial cells that express potent procoagulant tissue factor to circulating blood, causing thrombus formation leading to myocardial ishemia and other complications. Inflammatory markers such as CRP and IL-6 not only participate in lesion formation but also alter plaque architecture in favor of rupture.

Besides being recognized as a marker for disease and potential coronary problems, CRP appears to be an independent risk factor and directly promotes vascular inflammation. CRP is found in atheroscleotic plaques within foam cells and smooth muscle cells (Torzewski, 2000, Yasojima, 2001). It aids recruitment of monocytes into the arterial wall, promotes the uptake of native LDL by macrophages to form foam cells, and stimulates macrophages to produce the prothrombotic tissue factor and cytokines, TNFa, I1-1b and IL-6. CRP activates complement in atherosclerotic plaques. In endothelial cells CRP induces expression of adhesion molecules, such as vascular cell adhesion molecule-1 (VCAM-1), intracellular adhesion molecule-1 (ICAM-1) and E-selectin (10), and chemokine MCP-1. The pro-inflammatory effects of CRP in endothelial cells are implicated in part via IL-6 and endothelin-1. CRP sensitizes endothelial cells to destruction by cytotoxic CD4⁺ T cells. CRP has also been implicated in enhanced vasoreactivity in unstable plaques. Therefore, through several interrelated pathways, CRP exhibits deleterious effects on the vascular system and the stability of atheroscleotic plaques.

As referenced above, monocytes play a prominent role in vascular disease. Monocytes/macrophages inflammation seems to be pivotal in all stages of athersclerosis and acute coronary syndrome. As CRP is implicated in coronary syndromes, one possible route by which it exhibits an effect is by promoting various pro-inflammatory and pro-thrombotic gene expression in monocytes.

p38 MAP kinase appears to be implicated as a mediator between CRP and monocyte activity in the disease state. Accordingly, p38 antagonism in the context of CRP and vascular disease offers a novel therapy to treat underlying arterial inflammation and the complications associated with atherosclerosis including ischemia, myocardial infarction, unstable angina, stroke and sudden death.

In evaluating the role played by p38 in acute coronary disease, purified CRP was observed as activating or phosphorylating p38 kinase. As a result of p38 activation, the production and expression of certain inflammatory mediators from human peripheral blood monocytes (HPBMC's) is promoted. These effects are demonstrated below whereby CRP is observed to directly induce p38 MAP kinase activation in monocytes. In addition, CRP was also demonstrated to invoke IL-8, ET-1, COX-2, endothelin-1 and tissue factor in monocytes. This effect seems to be modulated through p38 kinase.

Pharmaceutical compositions useful for the present invention comprise an inhibitor of p38 MAP kinase as an active ingredient, a pharmaceutically acceptable carrier and optionally other therapeutic ingredients or adjuvants. The pharmaceutical compositions may be conveniently presented in unit dosage form and prepared by any of the methods well known in the art of pharmacy.

Any known route of administration may used in the present invention. The compositions or compounds useful in the present invention may be administered orally, parenterally, topically, rectally, nasally, vaginally, or via implanted reservior. Parenteral or systemic administration includes, but is not limited to, subcutaneous, intravenous, intraperitoneally, intramuscular, intra-articular, intra-synovial, intrastemol, intrathecal, intralesional, and intracranial injections. Preferably, the compositions or compounds of the present invention are administered parenterally.

The manner of administration and the formulation and dosage of the compounds useful in the invention depends on the nature of the condition, the severity of the condition, the particular subject to be treated, and the judgment of the practitioner; formulation and dosage will depend on mode of administration.

Inhibitors of p38 MAP Kinase

As used herein, the term “inhibitor” includes any suitable molecule, compound, formulation or substance that may regulate p38 MAP kinase activity. The inhibitor may be a protein or fragment thereof, a small molecule compound, or even a nucleic acid molecule. It may affect a single p38 MAP kinase isoform or more than one isoform of p38 MAP kinase. In a preferred embodiment of the invention, the inhibitor regulates the α isoform of p38 MAP kinase.

According to the present invention, the inhibitor may exhibit its regulatory effect upstream or downstream of p38 MAP kinase or on p38 MAP kinase directly. Examples of inhibitor regulated p38 activity include those where the inhibitor may decrease transcription and/or translation of p38 MAP kinase, may decrease or inhibit post-translational modification and/or cellular trafficking of p38 MAP kinase, or may shorten the half-life of p38 MAP kinase. The inhibitor may also reversibly or irreversibly bind p38 MAP kinase, inhibit its activation, inactivate its enzymatic activity, or otherwise interfere with its interaction with downstream substrates.

If acting on p38 MAP kinase directly, the inhibitor should exhibit an IC₅₀ value of about 10 μM or less, preferably 500 nm or less, more preferably 100 nm or less. In a related embodiment, the inhibitor should exhibit an IC₅₀ value relative to the p38 α isoform that is preferably at least ten fold less than that observed when the same inhibitor is tested against other p38 MAPK isoforms in the same or comparable assay.

To determine whether a candidate is an inhibitor useful for the invention, an evaluation can be done on its p38 MAP kinase activity as well as its relative IC₅₀ value. This evaluation can be accomplished through a variety of convential in vitro assays. Such assays include those that assess inhibition of kinase or ATPase activity of activated p38 MAP kinase. The assays may also assess the ability of the inhibitor to bind p38 MAP kinase or to reduce or block an identified downstream effect of activated p38 MAP kinase, e.g., cytokine secretion.

For example, conventional binding assays are fairly inexpensive and simple to run. As previously mentioned, binding of a molecule to p38 MAP kinase, in and of itself, may be inhibitory, due to steric, allosteric or charge-charge interactions. A binding assay can be performed in solution or on a solid phase using p38 MAP kinase or a fragment thereof as a target. By using this as an initial screen, one can evaluate libraries of compounds for potential p38 regulatory activity.

The target may be either free in solution, fixed to a support, expressed in or on the surface of a cell. A label (ie. radioactive, fluorescent, quenching, et cetera.) can be placed on the target, compound, or both to determine presence or absence of binding. This approach can also be used to conduct a competitive binding assay to assess the inhibition of binding of a target to a natural or artificial substrate or binding partner. In any case, one may measure, either directly or indirectly, the amount of free label versus bound label to determine binding. There are many known variations and adaptations of this approach to minimize interference with binding activity and optimize signal.

For purposes of in vitro cellular assays, the compounds that represent potential inhibitors of p38 MAP kinase function can be administered to a cell in any number of ways. Preferably, the compound or composition can be added to the medium in which the cell is growing, such as tissue culture medium for cells grown in culture. The compound is provided in standard serial dilutions or in an amount determined by analogy to known modulators. Alternatively, the potential inhibitor may be encoded by a nucleic acid that is introduced into the cell wherein the cell essentially produces the potential inhibitor itself.

Alternative assays involving in vitro analysis of potential inhibitors include those where cells (HeLa) transfected with DNA coding for relevant kinases can be activated with substances such as sorbitol, IL-1, TNF, or PMA (phorbol myristate acetate). After immunoprecipitation of cell lysates, equal aliquots of immune complexes of the kinases are pre-incubated for an adequate time with a specific concentration of the potential inhibitor followed by addition of kinase substrate buffer mix containing labeled ATP and GST-ATF2 or MBP. After incubation, kinase reactions are ceased by the addition of SDS loading buffer. Phosphorylated substrate is resolved through SDS-PAGE and visualized and quantitated in a phosphorimager. Both p38 regulation, in terms of phosphorylation, and IC₅₀ values can be determined by quantitation. See, for example Kumar,S., McDonnell, P., Gum, R., Hand, A., Lee, J., and Young, P. (1997) Biochem. Biophys. Res. Commun. 235, 533-538.

Other in vitro assays may also assess the production of TNF-A as a correlate to p38 MAP kinase activity. One such example is a human whole blood assay. In this assay, venous blood is collected from healthy male volunteers into a heparinized syringe and is used within 2 hours of collection. Test compounds are dissolved in 100% DMSO and 1 μl aliquots of drug concentrations ranging from 0 to 1 mM are dispensed into quadruplicate wells of a 24-well microtiter plate (Nunclon Delta SI, Applied Scientific, So. San Francisco, Calif.). Whole blood is added at a volume of 1 ml/well and the mixture is incubated for 15 minutes with constant shaking (Titer Plate Shaker, Lab-Line Instruments, Inc., Melrose Park, Ill.) at a humidified atmosphere of 5% CO₂ at 37° C. Whole blood is cultured either undiluted or at a final dilution of 1:10 with RPMI 1640 (Gibco 31800 +NaHCO₃, Life Technologies, Rockville, Md. and Scios, Inc., Sunnyvale, Calif.). At the end of the incubation period, 10 μl of LPS (E. coli 0111:B4, Sigma Chemical Co., St. Louis, Mo.) is added to each well to a final concentration of 1 or 0.1 μg/ml for undiluted or 1:10 diluted whole blood, respectively. The incubation is continued for an additional 2 hours. The reaction is stopped by placing the microtiter plates in an ice bath and plasma or cell-free supernates are collected by centrifugation at 3000 rpm for 10 minutes at 4° C. The plasma samples are stored at −80° C. until assayed for-TNF-α levels by ELISA, following the directions supplied by Quantikine Human TNF-α assay kit (R&D Systems, Minneapolis, Minn.). IC₅₀ values are calculated using the concentration of inhibitor that causes a 50% decrease as compared to a control.

A similar assay is an enriched mononuclear cell assay. The enriched mononuclear cell assay, begins with cryopreserved Human Peripheral Blood Mononuclear Cells (HPBMCs) (Clonetics Corp.) that are rinsed and resuspended in a warm mixture of cell growth media. The resuspended cells are then counted and seeded at 1×10⁶ cells/well in a 24-well microtitre plate. The plates are then placed in an incubator for an hour to allow the cells to settle in each well. After the cells have settled, the media is aspirated and new media containing 100 ng/ml of the cytokine stimulatory factor lipopolysaccharide (LPS) and a test chemical compound is added to each well of the microtiter plate. Thus, each well contains HPBMCs, LPS and a test chemical compound. The cells are then incubated for 2 hours, and the amount of the cytokine Tumor Necrosis Factor Alpha (TNF-α) is measured using an enzyme linked immunosorbent assay (ELISA). One such ELISA for detecting the levels of TNF-α is commercially available from R&D Systems. The amount of TNF-α production by the HPBMCs in each well is then compared to a control well to determine whether the chemical compound acts as an inhibitor of cytokine production.

IC₅₀ values are calculated using the concentration of inhibitor that causes a 50% decrease as compared to a control.

Exemplary Inhibitors

Compounds useful in the practice of the present invention include, but are not limited to, compounds of formula: 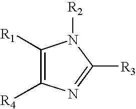
wherein

R₁ is a heteroaryl ring selected from 4-pyridyl, pyrimidinyl, quinolyl, isoquinolinyl, quinazolin-4-yl, 1-imidazolyl, 1-benzimidazolyl, 4-pyridazinyl, and a 1,2,4-triazin-5-yl ring, which heteroaryl ring is substituted one to three times with Y, N(R₁₀)C(O)R_{b}, a halo-substituted mono- or di-C₁₋₆ alkyl-substituted amino, or NHRₐ and which ring is further optionally substituted with C₁₋₄ alkyl, halogen, hydroxyl, optionally-substituted C₁₋₄ alkoxy, optionally-substituted C₁₋₄ alkylthio, optionally-substituted C₁₋₄ alkylsulfinyl, CH₂OR₁₂, amino, mono- and di-C₁₋₆ alkyl-substituted amino, NHFₐ, N(R₁₀)C(O)R_{b}, N(R₁₀)S(O)₂R_{d}, or an N-heterocyclyl ring which has from 5 to 7 members and optionally contains an additional heteroatom selected from oxygen, sulfur or NR₁₅;

Y is X₁-Rₐ;

X₁ is oxygen or sulfur;

Rₐ is C₁₋₆ alkyl, aryl, arylC₁₋₆ alkyl, heterocyclic, heterocyclylC₁₋₆ alkyl, heteroaryl, or heteroarylC₁₋₆ alkyl, wherein each of these moieties can be optionally substituted;

R_{b} is hydrogen, C₁₋₆ alkyl, C₃₋₇ cycloalkyl, aryl, arylC₁₋₄ alkyl, heteroaryl, heteroarylC₁₋₄ alkyl, heterocyclyl, or heterocyclylC₁₋₄ alkyl;

R_{d} is C₁₋₆ alkyl, C₃₋₇ cycloalkyl, aryl, arylC₁₋₄ alkyl, heteroaryl, heteroarylC₁₋₄ alkyl, heterocyclyl, or heterocyclylC₁₋₄ alkyl;

R₃ is hydrogen;

R₄ is phenyl, naphth-1-yl, naphth-2-yl, or a heteroaryl, which is optionally substituted by one or two substituents, each of which is independently selected, and which, for a 4-phenyl, 4-naphth-1-yl, 5-naphth-2-yl or 6-naphth-2-yl substituent, is halogen, cyano, nitro, —C(Z)NR₇R₁₇, —C(Z)OR₁₆, —(CR₁₀R₂₀)ᵥCOR₁₂, —SR₅, —SOR₅, —OR₁₂, halo-substituted-C₁₋₄ alkyl, C₁₋₄ alkyl, —ZC(Z)R₁₂, —NR₁₀C(Z)R₁₆, or —(CR₁₀R₂₀)ᵥNR₁₀R₂₀ and which, for other positions of substitution, is halogen, cyano, —C(Z)NR₁₃R₁₄, —C(Z)OR_{f}, —(CR₁₀R₂₀)_{m″}COR_{f}, —S(O)ₘR_{f}, —OR_{f}, —OR₁₂, halo-substituted C₁₋₄ alkyl, C₁₋₄ alkyl, —(CR₁₀R₂₀)_{m″}NR₁₀C(Z)R_{f}, —NR₁₀S(O)_{m′}R₈, —NR₁₀S(O)_{m′}NR₇R₁₇, —ZC(Z)R_{f}, —ZC(Z)R₁₂, or —(CR₁₀R₂₀)_{m″}NR₁₃R₁₄;

R_{f} is heterocyclyl, heterocyclylC₁₋₁₀ alkyl or R₈;

Z is oxygen or sulfur;

v is 0,1, or 2;

m is 0, 1, or 2;

m′ is 1 or 2;

m″ is 0, 1,2,3,4, or 5;

R₂ is C₁₋₁₀ alkyl N₃, —(CR₁₀R₂₀)_{n′}OR₉, heterocylyl, heterocycylC₁₋₁₀ alkyl, C₁₋₁₀ alkyl, halo-substituted C₁₋₁₀ alkyl, C₂₋₁₀ alkenyl, C₂₋₁₀ alkynyl, C₃₋₇ cycloalkyl, C₃₋₇ cycloalkylC₁₋₁₀ alkyl, C₅₋₇ cycloalkenyl, C₅₋₇cycloalkenylC₁₋₁₀ alkyl, aryl, arylC₁₋₁₀ alkyl, heteroaryl, heteroarylC₁₋₁₀ alkyl, (CR₁₀R₂₀)ₙOR₁₁, (CR₁₀R₂₀)ₙS(O)ₘR₁₈, (CR₁₀R₂₀)ₙNHS(O)₂R₁₈, (CR₁₀R₂₀)ₙNR₁₃R₁₄, (CR₁₀R₂₀)ₙNO₂, (CR₁₀R₂₀)ₙCN, (CR₁₀R₂₀)_{n′}SO₂R₁₈, (CR₁₀R₂₀)ₙS(O)_{m′}NR₁₃R₁₄, (CR₁₀R₂₀)ₙC(Z)R₁₁, (CR₁₀R₂₀)ₙOC(Z)R₁₁, (CR₁₀R₂₀)ₙC(Z)OR₁₁, (CR₁₀R₂₀)ₙC(Z)NR₁₃R₁₄, (CR₁₀R₂₀)ₙC(Z)NR₁₁OR₉, (CR₁₀R₂₀)ₙNR₁₀C(Z)R₁₁, (CR₁₀R₂₀)ₙNR₁₀C(Z)NR₁₃R₁₄, (CR₁₀R₂₀)ₙN(OR₆)C(Z)NR₁₃R₁₄, (CR₁₀R₂₀)ₙN(OR₆)C(Z)R₁₁, (CR₁₀R₂₀)ₙC(═NOR₆)R₁₁, (CR₁₀R₂₀)ₙNR₁₀C(═NR₁₉)NR₁₃R₁₄, (CR₁₀R₂₀)ₙOC(Z)NR₁₃R₁₄, (CR₁₀R₂₀)ₙNR₁₀C(Z)NR₁₃R₁₄, (CR₁₀R₂₀)ₙNR₁₀C(Z)OR₁₀, 5-(R₁₈)-1,2,4-oxadiazol-3-yl or 4-(R₁₂)-5-(R₁₈R₁₉)-4,5-dihydro-1,2,4-oxadiazol-3-yl; wherein the aryl, arylalkyl, heteroaryl, heteroaryl alkyl, cycloalkyl, cycloalkyl alkyl, heterocyclic and heterocyclic alkyl groups can be optionally substituted;

n is an integer having a value of 1 to 10;

n′ is 0, or an integer having a value of 1 to 10;

R₅ is hydrogen, C₁₋₄ alkyl, C₂₋₄ alkenyl, C₂₋₄ alkynyl or NR₇R₁₇, excluding the moieties ‥SR₅ being —SNR₇R₁₇ and —S(O)R₅ being —SOH;

R₆ is hydrogen, a pharmaceutically-acceptable cation, C₁₋₁₀ alkyl, C₃₋₇ cycloalkyl, aryl, arylC₁₋₄ alkyl, heteroaryl, heteroarylC₁₋₁₀ alkyl, heterocyclyl, aroyl, or C₁₋₁₀ alkanoyl;

R₇ and R₁₇ are each independently selected from hydrogen or C₁₋₄ alkyl, or R₇ and R₁₇ together with the nitrogen to which they are attached form a heterocyclic ring of 5 to 7 members which ring optionally contains an additional heteroatom selected from oxygen, sulfur or NR₁₅;

R₈ is C₁₋₁₀ alkyl, halo-substituted C₁₋₁₀ alkyl, C₂₋₁₀ alkenyl, C₂₋₁₀ alkynyl, C₃₋₇ cycloalkyl, C₅₋₇ cycloalkenyl, aryl, arylC₁₋₁₀ alkyl, heteroaryl, heteroarylC₁₋₁₀ alkyl, (CR₁₀R₂₀)ₙOR₁₁, (CR₁₀R₂₀)ₙS(O)ₘR₁₈, (CR₁₀R₂₀)ₙNHS(O)₂R₁₈, or (CR₁₀R₂₀)ₙNR₁₃R₁₄, wherein the aryl, arylalkyl, heteroaryl, and heteroaryl alkyl can be optionally substituted;

R₉ is hydrogen, —C(Z)R₁₁, optionally-substituted C₁₋₁₀ alkyl, S(O)₂R₁₈, optionally-substituted aryl or optionally-substituted arylC₁₋₄ alkyl;

R₁₀ and R₂₀ are each independently selected from hydrogen or C₁₋₄ alkyl;

R₁₁ is hydrogen, C₁₋₁₀ alkyl, C₃₋₇ cycloalkyl, heterocyclyl, heterocyclylC₁₋₁₀ alkyl, aryl, arylC₁₋₁₀ alkyl, heteroaryl or heteroarylC₁₋₁₀ alkyl;

R₁₂ is hydrogen or R₁₆;

R₁₃ and R₁₄ are each independently selected from hydrogen or optionally-substituted C₁₋₄ alkyl, optionally-substituted aryl or optionally-substituted arylC₁₋₄ alkyl, or together with the nitrogen to which they are attached form a heterocyclic ring of 5 to 7 members which ring optionally contains an additional heteroatom selected from oxygen, sulfur or NR₉;

R₁₅ is R₁₀ or C(Z)C₁₋₄ alkyl;

R₁₆ is C₁₋₄ alkyl, halo-substituted C₁₋₄ alkyl, or C₃₋₇ cycloalkyl;

R₁₈ is C₁₋₁₀ alkyl, C₃₋₇ cycloalkyl, heterocyclyl, aryl, arylC₁₋₁₀ alkyl, heterocyclyl, heterocyclylC₁₋₁₀ alkyl, heteroaryl or heteroarylC₁₋₁₀ alkyl; and

R₁₉ is hydrogen, cyano, C₁₋₄ alkyl, C₃₋₇ cycloalkyl or aryl;

or a pharmaceutically-acceptable salt thereof,

or wherein

R₁, Y, X₁, Rₐ, R_{b}, R_{d}, V, m, m′, m″, Z, n, n′, and R₅ are defined as above, and

R₂ is hydrogen, C₁₋₁₀ alkyl, halo-substituted C₁₋₁₀ alkyl, C₂₋₁₀ alkenyl, C₂₋₁₀ alkynyl, C₃₋₇ cycloalkyl, C₃₋₇ cycloalkylC₁₋₁₀ alkyl, C₅₋₇ cycloalkenyl, aryl, arylC₁₋₁₀ alkyl, heteroaryl, heteroarylC₁₋₁₀ alkyl, heterocyclyl, heterocyclylC₁₋₁₀ alkyl, (CR₁₀R₂₈)ₙOR₁₂, (CR₁₀R₂₈)_{n′}OR₁₃, (CR₁₀R₂₈)_{n′}S(O)ₘR₂₅, (CR₁₀R₂₈)ₙS(O)₂R₂₅, (CR₁₀R₂₈)_{n′}NHS(O)₂R₂₅, (CR₁₀R₂₈)_{n′}NR₈R₉, (CR₁₀R₂₈)_{n′}NO₂, (CR₁₀R₂₈)_{n′}CN, (CR₁₀R₂₈)_{n′}S(O)ₘNR₈R₉, (CR₁₀R₂₈)_{n′}C(Z)R₁₃, (CR₁₀R₂₈)_{n′}C(Z)OR₁₃, (CR₁₀R₂₈)_{n′}C(Z)NR₈R₉, (CR₁₀R₂₈)_{n′}C(Z)NR₁₃OR₁₂, (CR₁₀R₂₈)_{n′}NR₁₀C(Z)R₁₃, (CR₁₀R₂₈)_{n′}NR₁₀C(Z)NR₈R₉, (CR₁₀R₂₈)_{n′}N(OR₂₁)C(Z)NR₈R₉, (CR₁₀R₂₈)_{n′}N(OR₂₁)C(Z)R₁₃, (CR₁₀R₂₈)_{n′}C(═NOR₂₁)R₁₃, (CR₁₀R₂₈)_{n′}NR₁₀C(═NR₂₇)NR₈R₉, (CR₁₀R₂₈)_{n′}OC(Z)NR₈R₉, (CR₁₀R₂₈)_{n′}NR₁₀C(Z)OR₁₀, (CR₁₀R₂₈)_{n′}NR₁₀C(Z)OR₁₀, 5-(R₂₅)-1,2,4-oxadiazol-3-yl or 4-(R₁₂)-5-(R₁₈R₁₉)-4,5-dihydro-1,2,4-oxadiazol-3-yl; wherein the cycloalkyl, cycloalkyl alkyl, aryl, arylalkyl, heteroaryl, heteroarylalkyl, heterocyclyl, or heterocyclylalkyl moieties can be optionally substituted;

R₃ is hydrogen or Q-(Y₁)ₜ;

Q is an aryl or heteroaryl group;

t is 1, 2, or 3;

Y₁ is independently selected from hydrogen, C₁₋₅ alkyl, halo-substituted C₁₋₅ alkyl, halogen, or —(CR₁₀R₂₀)ₙY₂;

Y₂ is OR₈, NO₂, S(O)_{m″}R₁₁, SR₈, S(O)_{m″}OR₈, S(O)ₘNR₈R₉, NR₈R₉, O(CR₁₀R₂₀)_{n′}NR₈R₉, C(O)R₈, CO₂R₈, CO₂(CR₁₀R₂₀)_{n′}CONR₈R₉, ZC(O)R₈, CN, C(Z)NR₈R₉, NR₁₀C(Z)R₈, C(Z)NR₈OR₉, NR₁₀C(Z)NR₈R₉, NR₁₀S(O)_{m″}R₁₁, N(OR₂₁)C(Z)NR₈R₉, N(OR₂₁)C(Z)R, C(═NOR₂₁)R₈, NR₁₀C(═NR₁₅)SR₁₁, NR₁₀C(═NR₁₅)NR₈R₉, NR₁₀C(═CR₁₄R₂₄)SR₁₁, NR₁₀C(═CR₁₄R₂₄)NR₈R₉, NR₁₀C(O)C(O)NR₈R₉, NR₁₀C(O)C(O)OR₁₀, C(═NR₁₃)NR₈R₉, C(═NOR₁₃)NR₈R₉, C(═NR₁₃)ZR₁₁, OC(Z)NR₈R₈, NR₁₀S(O)_{m″}CF₃,NR₁₀C(Z)OR₁₀, 5-(R₁₈)-1,2,4-oxadiazol-3-yl or 4-(R₁₂)-5-(R₁₈R₁₉)-4,5-dihydro-1,2,4-oxadiazol-3-yl;

R₄ is phenyl, naphth-1-yl or naphth-2-yl which is optionally substituted by one or two substituents, each of which is independently selected, and which, for a 4-phenyl, 4-naphth-1-yl or 5-naphth-2-yl substituent, is halo, nitro, cyano, C(Z)NR₇R₁₇, C(Z)OR₂₃, (CR₁₀R₂₀)ᵥCOR₃₆, SR₅, SOR₅, OR₃₆, halo-substituted-C₁₋₄ alkyl, C₁₋₄ alkyl, ZC(Z)R₃₆, NR₁₀C(Z)R₂₃, or (CR₁₀R₂₀)ᵥNR₁₀R₂₀ and which, for other positions of substitution, is halo, nitro, cyano, C(Z)NR₁₆R₂₆, C(Z)OR₈, (CR₁₀R₂₀)_{m″}COR₈, S(O)ₘR₈, OR₈, halo-substituted-C₁₋₄ alkyl, C₁₋₄ alkyl, (CR₁₀R₂₀)_{m″}NR₁₀C(Z)R₈, NR₁₀S(O)_{m′}R₁₁, NR₁₀S(O)_{m′}NR₇R₁₇, ZC(Z)R₈ or (CR₁₀R₂₀)_{m″}NR₁₆R₂₆;

R₇ and R₁₇ are each independently selected from hydrogen or C₁₋₄ alkyl, or R₇ and R₁₇ together with the nitrogen to which they are attached form a heterocyclic ring of 5 to 7 members, which ring optionally contains an additional heteroatom selected from oxygen, sulfur or NR₂₂;

R₈ is hydrogen, heterocyclyl, heterocyclylalkyl or R₁₁;

R₉ is hydrogen, C₁₋₁₀ alkyl, C₂₋₁₀ alkenyl, C₂₋₁₀ alkynyl, C₃₋₇ cycloalkyl, C₅₋₇ cycloalkenyl, aryl, arylalkyl, heteroaryl or heteroarylalkyl, or R₈ and R₉ can together with the nitrogen to which they are attached form a heterocyclic ring of 5 to 7 members, which ring optionally contains an additional heteroatom selected from oxygen, sulfur or NR₁₂;

R₁₀ and R₂₀ are each independently selected from hydrogen or C₁₋₄ alkyl;

R₁₁ is C₁₋₁₀ alkyl, halo-substituted C₁₋₁₀ alkyl, C₂₋₁₀ alkenyl, C₂₋₁₀ alkynyl, C₃₋₇ cycloalkyl, C₅₋₇ cycloalkenyl, aryl, arylalkyl, heteroaryl or heteroarylalkyl;

R₁₂ is hydrogen, —C(Z)R₁₃ or optionally-substituted C₁₋₄ alkyl, optionally-substituted aryl, optionally-substituted arylC₁₋₄ alkyl, or S(O)₂R₂₅;

R₁₃ is hydrogen, C₁₋₁₀ alkyl, C₃₋₇ cycloalkyl, heterocyclyl, heterocyclylClilo alkyl, aryl, arylC₁₋₁₀ alkyl, heteroaryl or heteroaryl C₁₋₁₀ alkyl, wherein all of these moieties can be optionally substituted;

R₁₄ and R₂₄ are each independently selected from hydrogen, alkyl, nitro or cyano;

R₁₅ is hydrogen, cyano, C₁₋₄ alkyl, C₃₋₇ cycloalkyl or aryl;

R₁₆ and R₂₆ are each independently selected from hydrogen or optionally-substituted C₁₋₄ alkyl, optionally-substituted aryl or optionally-substituted arylC₁₋₄ alkyl, or together with the nitrogen to which they are attached form a heterocyclic ring of 5 to 7 members, which ring optionally contains an additional heteroatom selected from oxygen, sulfur or NR₁₂;

R₁₈ and R₁₉ are each independently selected from hydrogen, C₁₋₄ alkyl, substituted alkyl, optionally-substituted aryl, optionally-substituted arylalkyl, or together denote an oxygen or sulfur;

R₂₁ is hydrogen, a pharmaceutically-acceptable cation, C₁₋₁₀ alkyl, C₃₋₇ cycloalkyl, aryl, arylC₁₋₄ alkyl, heteroaryl, heteroarylalkyl, heterocyclyl, aroyl, or C₁₋₁₀ alkanoyl;

R₂₂ is R₁₀ or C(Z)—C₁₋₄ alkyl;

R₂₃ is C₁₋₄ alkyl, halo-substituted-C₁₋₄ alkyl, or C₃₋₅ cycloalkyl;

R₂₅ is C₁₋₁₀ alkyl, C₃₋₇ cycloalkyl, heterocyclyl, aryl, arylalkyl, heterocyclyl, heterocyclylC₁₋₁₀ alkyl, heteroaryl or heteroarylalkyl;

R₂₇ is hydrogen, cyano, C₁₋₄ alkyl, C₃₋₇ cycloalkyl, or aryl;

R₂₈ is hydrogen, C₁₋₆ alkyl, C₃₋₇ cycloalkyl, aryl, arylC₁₋₄ alkyl, heteroaryl, heteroarylC₁₋₄alkyl, heterocyclyl, or heterocyclylC₁₋₄ alkyl moiety, all of which can be optionally substituted; and

R₃₆ is hydrogen or R₂₃;

or a pharmaceutically acceptable salt thereof

Exemplary compounds of the above formula include but are not limited to:
1-[3-(4-morpholinyl)propyl]-4-(4-fluorophenyl)-5-(4-pyridyl)imidazole;
1-(3-chloropropyl)-4-(4-fluorophenyl)-5-(4-pyridyl)imidazole;
1-(3-azidopropyl)-4-(4-fluorophenyl)-5-(4-pyridyl)imidazole;
1-(3-aminopropyl)-4-(4-fluorophenyl)-5-(4-pyridyl)imidazole;
1-(3-methylsulfonamidopropyl)-4-(4-fluorophenyl)-5-(4-pyridyl)imidazole;
1-[3-(N-phenylmethyl)aminopropyl]-4-(4-fluorophenyl)-5-(4-pyridyl)imidazole;
1-[3-(N-phenylmethyl-N-methyl)aminopropyl]-4-(4-fluorophenyl)-5-(4-pyridyl)imidazole;
1-[3-(1-pyrrolidinyl)propyl]-4-(4-fluorophenyl)-5-(4-pyridyl)imidazole;
1-(3-diethylaminopropyl)-4-(4-fluorophenyl)-5-(4-pyridyl)imidazole;
1-[3-(1-piperidinyl)propyl]-4-(4-fluorophenyl)-5-(4-pyridyl)imidazole;
1-[3-(methylthio)propyl]-4-(4-fluorophenyl)-5-(4-pyridyl)imidazole;
1-[2-(4-morpholinyl)ethyl]-4-(4-fluorophenyl)-5-(4-pyridyl)imidazole;
1-[3-(4-morpholinyl)propyl]-4-(3-methylthiophenyl)-5-(4-pyridyl)imidazole;
(+/−)-1-[3-(4-morpholinyl)propyl]-4-(3-methylsulfinylphenyl)-5-(4-pyridyl)imidazole;
1-[3-(N-methyl-N-benzyl)aminopropyl]-4-(3-methylthiophenyl)-5-(4-pyridyl)imidazole;
1-[3-(N-methyl-N-benzyl)aminopropyl]-4-(3-methylsulfinylphenyl)-5-(4-pyridyl)imidazole;
1-[4-(methylthio)phenyl]-4-(4-fluorophenyl)-5-(4-pyridyl)imidazole;
1-[4-(methylsulfinyl)phenyl]-4-(4-fluorophenyl)-5-(4-pyridyl)imidazole;
1-[3-(methylthio)phenyl]-4-(4-fluorophenyl)-5-(4-pyridyl)imidazole;
(+/−)-1-[3-(methylsulfinyl)phenyl]-4-(4-fluorophenyl)-5-(4-pyridyl)imidazole;
1-[2-(methylthio)phenyl]-4-(4-fluorophenyl)-5-(4-pyridyl)imidazole;
1-[2-(methylsulfinyl)phenyl]-4-(4-fluorophenyl)-5-(4-pyridyl)imidazole;
1-[4-(4-morpholinyl)butyl]-4-(4-fluorophenyl)-5-(4-pyridyl)imidazole;
1-cyclopropyl-4-(4-fluorophenyl)-5-(4-pyridyl)imidazole;
1-isopropyl-4-(4-fluorophenyl)-5-(4-pyridyl)imidazole;
1-cyclopropylmethyl-4-(4-fluorophenyl)-5-(4-pyridyl)imidazole;
1-tert-butyl-4-(4-fluorophenyl)-5-(4-pyridyl)imidazole;
1-(2,2-diethoxyethyl)-4-(4-fluorophenyl)-5-(4-pyridyl)imidazole;
1-formylmethyl-4-(4-fluorophenyl)-5-(4-pyridyl)imidazole;
1-hydroxyiminylmethyl-4-(4-fluorophenyl)-5-(4-pyridyl)imidazole;
1-cyanomethyl-4-(4-fluorophenyl)-5-(4-pyridyl)imidazole;
1-[3-(4-morpholinyl)propyl)-4-(4-fluorophenyl)-5-(2-methylpyrid-4-yl)imidazole;
4-(4-fluorophenyl)-1-[3-(4-morpholinyl)propyl]-5-(2-chloropyridin-4-yl)imidazole;
4-(4-fluorophenyl)-1-[3-(4-morpholinyl)propyl]-5-(2-amino-4-pyridinyl)imidazole;
1-(4-carboxymethyl)propyl-4-(4-fluorophenyl)-5-(4-pyridyl)imidazole;
1-(4-carboxypropyl)-4-(4-fluorophenyl)-5-(4-pyridyl)imidazole;
1-(3-carboxymethyl)ethyl-4-(4-fluorophenyl)-5-(4-pyridyl)imidazole;
1-(3-carboxy)ethyl-4-(4-fluorophenyl)-5-(4-pyridyl)imidazole;
1-(1-benzylpiperidin-4-yl)-4-(4-fluorophenyl)-5-(4-pyridyl)imidazole;
5-(2-aminopyrimidin-4-yl)-4-(4-fluorophenyl)-1-[3-(4-morpholinyl)propyl]imidazole;
5-(2-aminopyrimidin-4-yl)-4-(4-fluorophenyl)-1-(1-benzylpiperidin-4-yl)imidazole;
5-(2-aminopyrimidin-4-yl)-4-(4-fluorophenyl)-1-(2-propyl)imidazole;
5-(2-aminopyrimidin-4-yl)-4-(4-fluorophenyl)-1-(cyclopropylmethyl)imidazole;
5-(2-aminopyrimidin-4-yl)-4-(4-fluorophenyl)-1-(1-carboxyethyl-4-piperidinyl)imidazole;
5-(2-aminopyrimidin-4-yl)-4-(4-fluorophenyl)-1-(4-piperidinyl)imidazole;
1-methyl-4-phenyl-5-(4-pyridyl)imidazole;
1-methyl-4-[3-(chlorophenyl)]-5-(4-pyridinyl)imidazole;
1-methyl-4-(3-methylthiophenyl)-5-(4-pyridyl)imidazole;
(+/−)-1-methyl-4-(3-methylsulfinylphenyl)-5-(4-pyridyl)imidazole;
(+/−)-4-(4-fluorophenyl)-1-[3-(methylsulfinyl)propyl]-5-(4-pyridinyl)imidazole;
4-(4-fluorophenyl)-1-[(3-methylsulfonyl)propyl]-5-(4-pyridinyl)imidazole;
1-(3-phenoxypropyl)-4-(4-fluorophenyl)-5-(4-pyridinyl)imidazole;
1-[3-(phenylthio)propyl]-4-(4-fluorophenyl)-5-(4-pyridinyl)imidazole;
1-[3-(4-morpholinyl)propyl]-4-(4-fluorophenyl)-5-(4-quinolyl)imidazole;
(+/−)-1-(3-phenylsulfinylpropyl)-4-(4-fluorophenyl)-5-(4-pyridinyl)imidazole;
1-(3-ethoxypropyl)-4-(4-fluorophenyl)-5-(4-pyridinyl)imidazole;
1-(3-phenylsulfonylpropyl-4-(4-fluorophenyl)-5-(4-pyridinyl)imidazole;
1-[3-(4-morpholinyl)propyl]-4-(3-chlorophenyl)-5-(4-pyridyl)imidazole;
1-[3-(4-morpholinyl)propyl]-4-(3,4-dichlorophenyl)-5-(4-pyridyl)imidazole;
4-[4-(4-fluorophenyl)-1-[3-(4-morpholinyl)propyl]-5-(pyrimid-2-one-4-yl)imidazole;
4-(4-fluorophenyl)-5-[2-(methylthio)-4-pyrimidinyl]-1-[3-(4-morpholinyl)propyl]imidazole;
(+/−)-4-(4-fluorophenyl)-5-[2-(methylsulfinyl)-4-pyrimidinyl]-1-[3-(4-morpholinyl)propyl]imidazole;
1-(1-propenyl)-4-(4-fluorophenyl)-5-(4-pyridinyl)imidazole;
1-(2-propenyl)-4-(4-fluorophenyl)-5-(4-pyridinyl)imidazole;
5-[(2-N,N-dimethylamino)pyrimidin-4-yl]-4-(4-fluorophenyl)-1-[3-(4-morpholinyl)propyl]imidazole;
1-[3-(4-morpholinyl)propyl]-5-(4-pyridinyl)-4-[4-(trifluoromethyl)phenyl]imidazole;
1-[3-(4-morpholinyl)propyl]-5-(4-pyridinyl)-4-[3-(trifluoromethyl)phenyl]imidazole;
1-(cyclopropylmethyl)-4-(3,4-dichlorophenyl)-5-(4-pyridinyl)imidazole;
1-(cyclopropylmethyl)-4-(3-trifluoromethylphenyl)-5-(4-pyridinyl)imidazole;
1-(cyclopropylmethyl)-4-(4-fluorophenyl)-5-(2-methylpyrid-4-yl)imidazole;
1-[3-(4-morpholinyl)propyl]-5-(4-pyridinyl)-4-(3 ,5-bistrifluoromethylphenyl)imidazole;
5-[4-(2-aminopyrimidinyl)]-4-(4-fluorophenyl)-1-(2-carboxy-2,2-dimethylethyl)imidazole;
1-(1-formyl-4-piperidinyl)-4-(4-fluorophenyl)-5-(4-pyridinyl)imidazole;
5-(2-amino-4-pyrimidinyl)-4-(4-fluorophenyl)-1-(1-methyl-4-piperidinyl)imidazole;
1-(2,2-dimethyl-3-morpholin-4-yl)propyl-4-(4-fluorophenyl)-5-(2-amino-4-pyrimidinyl)imidazole;
4-(4-fluorophenyl)-5-(4-pyridyl)-1-(2-acetoxyethyl)imidazole;
5-(2-aminopyrimidin-4-yl)-4-(4-fluorophenyl)-1-(1-benzylpyrrolin-3-yl)imidazole;
5-(2-aminopyrimidin-4-yl)-4-(4-fluorophenyl)-1-(2,2,6,6-tetramethylpiperidin-4-yl)imidazole;
5-[4-(2-N-methylamino)pyrimidinyl]-4-(4-fluorophenyl)-1-(4-N-methylpiperidine)imidazole;
5-[4-(2-N-methylamino)pyrimidinyl]-4-(4-fluorophenyl)-1-(4-N-morpholino-1-propyl)imidazole;
5-[4-(2-N-methylamino)pyrimidinyl)-4-(4-fluorophenyl)-1-(4-piperidine)imidazole;
5-[(2-ethylamino)pyrimidin-4-yl]-4-(4-fluorophenyl)-1-(1-methylpiperidin-4-yl)imidazole;
4-(4-fluorophenyl)-5-[2-(isopropyl)aminopyrimidin-4-yl]-1-(1-methylpiperidin-4-yl)imidazole;
5-(2-acetamido-4-pyrimidinyl)-4-(4-fluorophenyl)-1-(4-N-morpholino-1-propyl)imidazole;
5-(2-acetamido-4-pyrimidinyl)-4-(4-fluorophenyl)-1-(1-methyl-4-piperidinyl)imidazole;
5-[4-(2-N-methylthio)pyrimidinyl]-4-(4-fluorophenyl)-1-(4-piperidine)imidazole;
4-(fluorophenyl)-1-(methyl-4-piperidinyl)-5-(2-methylthio-4-pyrimidinyl)imidazole;
4-(fluorophenyl)-1-(methyl-4-piperidinyl)-5-(2-methysulfinyl-4-pyrimidinyl)imidazole;
1-tert-butyl-4-(4-fluorophenyl)-5-(2-methysulfinyl-4-pyrimidinyl)imidazole;
5-[4-(2-aminopyrimidinyl)]-4-(4-fluorophenyl)-1-(2,2,6,6-tetramethyl-4-piperidinyl)imidazole;
5-[4-(2-N-methylamino-4-pyrimidinyl)]-4-(4-fluorophenyl)-1-(2,2,6,6-tetramethyl-4-piperidine)imidazole;
5-(2-amino-4-pyrimidinyl)-4-(4-fluorophenyl)-1-(tetrahydro-4-thiopyranyl)imidazole;
5-(2-amino-4-pyrimidinyl)-4-(4-fluorophenyl)-1-(tetrahydro-4-pyranyl)imidazole;
5-(2-methylamino-4-pyrimidinyl)-4-(4-fluorophenyl)-1-(2-cyanoethyl)imidazole;
5-(2-amino-4-pyrimidinyl)-4-(4-fluorophenyl)-1-(tetrahydro-4-sulfinylpyranyl)imidazole;
5-(2-amino-4-pyrimidinyl)-4-(4-fluorophenyl)-1-(tetrahydro-4-sulfonylpyranyl)imidazole;
5-(2-methylamino-4-pyrimidinyl)-4-(4-fluorophenyl)-1-(2,2,2-trifluoroethyl-4-piperidinyl)imidazole;
5-(2-amino-4-pyrimidinyl)-4-(4-fluorophenyl)-1-(trifluoroacetyl-4-piperidinyl)imidazole;
5-(4-pyridyl)-4-(4-fluorophenyl)-1-(4-piperidinyl)imidazole;
5-(4-pyridyl)-4-(4-fluorophenyl)-1-(1-t-butoxycarbonyl-4-piperidinyl)imidazole;
5-(2-amino-4-pyrimidinyl)-4-(4-fluorophenyl)-1-(4-(1,3-dioxycyclopentyl)cyclohexyl)imidazole;
5-(2-amino-4-pyrimidinyl)-4-(4-fluorophenyl)-1-(4-ketocyclohexyl)imidazole;
5-(2-amino-4-pyrimidinyl)-4-(4-fluorophenyl)-1-(4-cyclohexyl oxime) imidazole;
5-(2-amino-4-pyrimidinyl)-4-(4-fluorophenyl)-1-(4-cyclohexyl hydroxylamine) imidazole;
5-(2-amino-4-pyrimidinyl)-4-(4-fluorophenyl)-1-(trans-4-hydroxyurea) imidazole;
5-(2-amino-4-pyrimidinyl)-4-(4-fluorophenyl)-1-(cis-4-hydroxyurea) imidazole;
5-(2-amino-4-pyrimidinyl)-4-(4-fluorophenyl)-1-(4-hydroxycyclohexyl)imidazole;
5-[4-(2-N-methylamino)pyrimidinyl]-4-(4-fluorophenyl)-1-(4-ketocyclohexyl)imidazole;
5-[4-(2-N-methylamino)pyrimidinyl]-4-(4-fluorophenyl)-1-(trans-4-hydroxy-cyclohexyl)imidazole;
5-[4-(2-N-methylamino)pyrimidinyl]-4-(4-fluorophenyl)-1-(cis-4-hydroxycyclohexyl)imidazole;
5-[4-(2-N-methylamino)pyrimidinyl]-4-(4-fluorophenyl)-1-[4-(cis-pyrrolidinyl)cyclohexyl]imidazole;
5-[4-(2-N-methylamino)pyrimidinyl]-4-(4-fluorophenyl)-1-[4-(trans-1-pyrrolidinyl)cyclohexyl]imidazole;
5-[4-(2-N-methylamino)pyrimidinyl]-4-(4-fluorophenyl)-1-(4-ethynyl-4-hydroxy-cyclohexyl)imidazole;
5-[4-(2-N-methylamino)pyrimidinyl]-4-(4-fluorophenyl)-1-(4-(1-propynyl)-4-hydroxycyclohexyl)imidazole;
5-[4-(2-N-methylamino)pyrimidinyl]-4-(4-fluorophenyl)-1-(4-amino-4-methyl-cyclohexyl)imidazole;
5-[4-(2-N-methylamino)pyrimidinyl]-4-(4-fluorophenyl)-1-(4-acetamido-4-methylcyclohexyl)imidazole;
5-[4-(2-N-methylamino)pyrimidinyl]-4-(4-fluorophenyl)-1-(4-hydroxy-4-methylcyclohexyl)imidazole;
5-[4-(2-N-methylamino)pyrimidinyl]-4-(4-fluorophenyl)-1-(4-oxiranylcyclohexyl)imidazole;
5-[4-(2-N-methylamino)pyrimidinyl]-4-(4-fluorophenyl)-1-(4-cyanomethyl-4-hydroxycyclohexyl)imidazole;
5-[4-(2-N-methylamino)pyrimidinyl]-4-(4-fluorophenyl)-1-(4-hydroxy-4-hydroxymethylcyclohexyl)imidazole;
5-[4-(2-amino)pyrimidinyl]-4-(4-fluorophenyl)-1-[4-hydroxy-4-(1-propynyl)-cyclohexyl]imidazole;
5-[4-(2-amino)pyrimidinyl]-4-(4-fluorophenyl)-1-(4-hydroxy-4-methylcyclohexyl)imidazole;
5-[4-(2-N-methylamino)pyrimidinyl]-4-(4-fluorophenyl)-1-(4-hydroxy-4-isopropyl-cyclohexyl)imidazole;
5-[4-(2-N-methylamino)pyrimidinyl]-4-(4-fluorophenyl)-1-(4-hydroxy-4-phenyl-cyclohexyl)imidazole;
5-[4-(2-N-methylamino)pyrimidinyl]-4-(4-fluorophenyl)-1-(4-hydroxy-4-benzyl-cyclohexyl)imidazole;
5-[4-(2-N-methylamino)pyrimidinyl]-4-(4-fluorophenyl)-1-(4-hydroxy-4-cyanomethyl cyclohexyl)imidazole;
5-[4-(2-N-methylamino)pyrimidinyl]-4-(4-fluorophenyl)-1-(4-hydroxy-4-(2-cyanoethyl)cyclohexyl)imidazole;
5-[4-(2-N-methylamino)pyrimidinyl]-4-(4-fluorophenyl)-1-(4-hydroxy-4-(2-aminoethyl)cyclohexyl)imidazole;
5-[4-(2-N-methylamino)pyrimidinyl]-4-(4-fluorophenyl)-1-(4-hydroxy-4-(2-nitroethyl)-cyclohexyl)imidazole;
5-[4-(2-N-methylamino)pyrimidinyl]-4-(4-fluorophenyl)-1-(4-hydroxymethyl-4-amino-cyclohexyl)imidazole;
5-[4-(2-N-methylamino)pyrimidinyl]-4-(4-fluorophenyl)-1-(4-hydroxy-4-amino-cyclohexyl)imidazole;
5-[4-(2-N-methylamino)pyrimidinyl]-4-(4-fluorophenyl)-1-(4-aminocyclohexyl)imidazole;
5-[4-(2-N-methylamino)pyrimidinyl]-4-(4-fluorophenyl)-1-(4-hydroxy-4-thiomethyl cyclohexyl)imidazole;
5-[4-(2-N-methylamino)pyrimidinyl]-4-(4-fluorophenyl)-1-(4-hydroxy-4-hydroxy methylcyclohexyl)imidazole;
5-[4-(2-N-methylamino)pyrimidinyl]-4-(4-fluorophenyl)-1-(4-hydroxy-4-aminomethylcyclohexyl)imidazole;
5-[4-(2-amino)pyrimidinyl]-4-(4-fluorophenyl)-1-(4-amino-4-methyl-cyclohexyl)imidazole;
5-[4-(2-amino)pyrimidinyl]-4-(4-fluorophenyl)-1-(4-hydroxy-4-methyl-cyclohexyl)imidazole;
5-[4-(2-amino)pyrimidinyl]-4-(4-fluorophenyl)-1-(4-oxiranylcyclohexyl)imidazole;
4-(fluorophenyl)-1-(methyl-4-piperidinyl)-5-(2-methysulfinyl-4-pyrimidinyl)imidazole;
4-(fluorophenyl)-1-(methyl-4-piperidinyl)-5-(2-methylthio-4-pyrimidinyl)imidazole;
5-[(2-benzylamino)pyrimidin-4-yl]-4-(4-fluorophenyl)-1-(1-methylpiperidin-4-yl)imidazole;
4-(4-fluorophenyl)-1-(1-methylpiperidin-4-yl)-5-[2-(4-tetrahydrothiopyranyl)aminopyrimidin-4-yl]imidazole;
4-(4-fluorophenyl)-5-[(2-hydroxy)ethylamino]pyrimidin-4-yl-1-(1-methylpiperidin-4-yl)imidazole;
5-[(2-(3-chlorobenzylamino)pyrimidin-4-yl]-4-(4-fluorophenyl)-1-(1-methylpiperidin-4-yl)imidazole;
5-[(2-(1-naphthylmethylamino)pyrimidin-4-yl]-4-(4-fluorophenyl)-1-(1-methylpiperidin-4-yl)imidazole;
5-[(2-(1-benzyl-4-piperidinylamino)pyrimidin-4-yl]-4-(4-fluorophenyl)-1-(1-methylpiperidin-4-yl)imidazole;
4-(4-fluorophenyl)-1-(1-methylpiperidin-4-yl)-5-[2-[3-(morpholino)propyl]aminopyrimidin-4-yl]imidazole;
5-[2-[(3-bromophenyl)amino]pyrimidin-4-yl]-4-(4-fluorophenyl)-1-(1-methylpiperidin-4-yl)imidazole;
5-[(2-(piperonylamino)pyrimidin-4-yl]-4-(4-fluorophenyl)-1-(1-methylpiperidin-4-yl)imidazole;
5-[(2-(4-piperidinylamino)pyrimidin-4-yl]-4-(4-fluorophenyl)-1-(1-methylpiperidin-4-yl)imidazole;
5-[(2-(5-chlorotryptamino)pyrimidin-4-yl]-4-(4-fluorophenyl)-1-(1-methylpiperidin-4-yl)imidazole;
5-[(2-(2,2,6,6-tetramethylpiperidin-4-yl)aminopyrimidin-4-yl]-4-(4-fluorophenyl)-1-(1-methylpiperidin-4-yl)imidazole;
5-[(2-[1-ethoxycarbonyl)piperidin-4-yl]aminopyrimidin-4-yl]-4-(4-fluorophenyl)-1-(1-methylpiperidin-4-yl)imidazole;
1-(4-oxocyclohexyl)-4-(4-fluorophenyl)-5-[(2-methoxy)pyrimidin-4-yl]imidazole;
cis-1-(4-hydroxycyclohexyl)-4-(4-fluorophenyl)-5-[(2-methoxy)pyrimidin-4-yl]imidazole;
trans-1-(4-hydroxycyclohexyl)-4-(4-fluorophenyl)-5-[(2-methoxy)pyrimidin-4-yl]imidazole;
1-(4-oxocyclohexyl)-4-(4-fluorophenyl)-5-[(2-methylthio)pyrimidin-4-yl]imidazole;
trans-1-(4-hydroxycyclohexyl)-4-(4-fluorophenyl)-5-[(2methylthio)pyrimidin-4-yl]imidazole;
1-(4-oxocyclohexyl)-4-(4-fluorophenyl)-5-[(2-hydroxy)pyrimidin-4-yl]imidazole;
1-(4-oxocyclohexyl)-4-(4-fluorophenyl)-5-[(2-isopropoxy)pyrimidin-4-y]imidazole;
1-(4-hydroxycyclohexyl)-4-(4-fluorophenyl)-5-[(2-isopropoxy)pyrimidin-4-yl]imidazole;
trans-1-(4-hydroxy-4-methylcyclohexyl)-4-(4-fluorophenyl)-5-[(2-methoxy)pyrimidin-4-yl]imidazole;
cis-1-(4-hydroxy-4-methylcyclohexyl)-4-(4-fluorophenyl)-5-[(2-methoxy)pyrimidin-4-yl]imidazole;
trans-1-(4-hydroxycyclohexyl)-4-(4-fluorophenyl)-5-[(2-ethoxy)pyrimidin-4-yl]imidazole;
1-(4-piperidinyl)-4-(4-fluorophenyl)-5-(2-phenoxypyrimidin-4-yl)imidazole;
1-(4-piperidinyl)-4-(4-fluorophenyl)-5-(2-phenoxy-4-pyridinyl)imidazole;
1-(4-piperidinyl)-4-(4-fluorophenyl)-5-[2-(4-methoxyphenoxy)-4-pyridinyl]imidazole;
1-(4-piperidinyl)-4-(4-fluorophenyl)-5-[2-(4-fluorophenoxy)-4-pyridinyl]imidazole;
1-(piperidin-4-yl)-4-(4-fluorophenyl)-5-[2-(4-methoxyphenoxy)pyrimidin-4-yl]imidazole;
1-(piperidin-4-yl)-4-(4-fluorophenyl)-5-[2-(4-fluorophenoxy)pyrimidin-4-yl]imidazole;
1-(piperidin-4-yl)-4-(4-fluorophenyl)-5-[2-(4-aminocarbonylphenoxy)pyrimidin-4-yl]imidazole;
1-(piperidin-4-yl)-4-(4-fluorophenyl)-5-[2-(4-ethylphenoxy)pyrimidin-4-yl]imidazole;
1-(piperidin-4-yl)-4-(4-fluorophenyl)-5-[2-(4-benzyloxyphenoxy)pyrimidin-4-yl]imidazole;
1-(piperidin-4-yl)-4-(4-fluorophenyl)-5-[2-(4-cyanophenoxy)pyrimidin-4-yl]imidazole;
1-(piperidin-4-yl)-4-(4-fluorophenyl)-5-[2-(4-hydroxyphenoxy)pyrimidin-4-yl]imidazole;
1-(4-hydroxycyclohexyl)-4-(4-fluorophenyl)-5-[2-(phenoxy)pyrimidin-4-yl]imidazole;
1-(piperidin-4-yl)-4-(4-fluorophenyl)-5-[2-(2,6-dimethylphenoxy)pyridin-4-yl]imidazole;
1-(piperidin-4-yl)-4-(4-fluorophenyl)-5-[2-(4-methylphenoxy)pyridin-4-yl]imidazole;
1-(piperidin-4-yl)-4-(4-fluorophenyl)-5-[2-(4-chlorophenoxy)pyridin-4-yl]imidazole;
1-[3-(N-morpholino)propyl]-4-(4-fluorophenyl)-5-[2-(phenoxy)pyrimidin-4-yl]imidazole;
1-(piperidin-4-yl)-4-(4-fluorophenyl)-5-[2-(3-methoxyphenoxy)pyrimidin-4-yl]imidazole;
1-(piperidin-4-yl)-4-(4-fluorophenyl)-5-[2-(4-phenylphenoxy)pyrimidin-4-yl]imidazole;
1-(piperidin-4-yl)-4-(4-fluorophenyl)-5-[2-(4-phenoxyphenoxy)pyrimidin-4-yl]imidazole;
1-(piperidin-4-yl)-4-(4-fluorophenyl)-5-[2-(3-hydroxyphenoxy)pyrimidin-4-yl]imidazole;
1-(3-(N-morpholino)propyl)-4-(4-fluorophenyl)-5-[2-(4-fluorophenoxy)pyrimidin-4-yl]imidazole;
1-(piperidin-4-yl)-4-(4-fluorophenyl)-5-[2-(2-hydroxyphenoxy)pyrimidin-4-yl]imidazole;
1-(piperidin-4-yl)-4-(4-fluorophenyl)-5-[2-((3,4-methylenedioxy)phenoxy)pyrimidin-4-yl]imidazole;
1-(piperidin-4-yl)-4-(4-fluorophenyl)-5-[2-(3-fluorophenoxy)pyrimidin-4-yl]imidazole;
1-(piperidin-4-yl)-4-(4-fluorophenyl)-5-[2-(2-fluorophenoxy)pyrimidin-4-yl]imidazole;
1-(piperidin-4-yl)-4-(4-fluorophenyl)-5-[2-(2-methoxyphenoxy)pyrimidin-4-yl]imidazole;
1-(piperidin-4-yl)-4-(4-fluorophenyl)-5-[2-(3-trifluoromethylphenoxy)pyrimidin-4-yl]imidazole;
1-(piperidin-4-yl)-4-(4-fluorophenyl)-5-[2-(3,4-difluorophenoxy)pyrimidin-4-yl]imidazole;
1-(piperidin-4-yl)-4-(4-fluorophenyl)-5-[2-(4-methylsulfonylphenoxy)pyrimidin-4-yl]imidazole;
1-(4-piperidinyl)-4-(4-fluorophenyl)-5-(2-thiophenoxypyrimidin-4yl)imidazole;
1-(4-piperidinyl)-4-(4-fluorophenyl)-5-[2-(1-methyltetrazol-5-ylthio)pyridin-4-yl]imidazole;
5-[2-(2-hydroxyethoxy)pyrimidin-4-yl]-4-(4-fluorophenyl)-1-(4-oxocyclohexyl)imidazole;
5-[2-(2-hydroxyethoxy)]pyrimidin-4-yl)-4-(4-fluorophenyl)-1-(4-hydroxycyclohexyl)imidazole;
5-[2-(2-tert-butylamino)ethoxypyrimidin-4-yl]-4-(4-fluorophenyl)-1-(4-oxocyclohexyl)imidazole;
5-[2-(2-tert-butylamino)ethoxypyrimidin-4-yl]-4-(4-fluorophenyl)-1-(4-hydroxycyclohexyl)imidazole;
1-(4-piperidinyl)-4-(4-Fluorophenyl)-5-(2-isopropoxy-4-pyrimidinyl)imidazole;
1-(4-piperidinyl)-4-(4-Fluorophenyl)-5-(2-methoxy-4-pyrimidinyl)imidazole;
5-(2-hydroxy-4-pyrimidinyl)-4-(4-fluorophenyl)-1-(4-piperidinyl)imidazole;
5-(2-methoxy-4-pyridinyl)-4-(4-fluorophenyl)-1-(4-piperidinyl)imidazole;
5-(2-isopropoxy-4-pyridinyl)-4-(4-fluorophenyl)-1-(4-piperidinyl)imidazole;
5-(2-methylthio-4-pyrimidinyl)-4-(4-fluorophenyl)-1-(4-piperidinyl)imidazole;
5-(2-methylthio-4-pyrimidinyl)-4-(4-fluorophenyl)-1-[1-methyl-4-piperidinyl]imidazole;
5-(2-ethoxy-4-pyrimidinyl)-4-(4-fluorophenyl)-1-(4-piperidinyl)imidazole;
1-(1-ethylcarboxylpiperidin-4-yl)-3-(4-thiomethylphenyl)-5-[2-(thiomethyl)pyrimidin-4-yl]-imidazole;
1-(1-ethylcarbonylpiperidin-4-yl)-4-(4-methylsulfinylphenyl)-5-[(2-methylsulfinyl)pyrimidin-4-yl]imidazole;
2-(4-methylthiophenyl)-4-(4-fluorophenyl)-5-(2-methoxy-4-pyrimidinyl)imidazole;
2-(4-methylsulfinylphenyl)-4-(4-fluorophenyl)-5-(2-methoxy-4-pyrimidinyl)imidazole;
2-[(4-N,N-dimethyl)aminomethylphenyl]-4-(4-fluorophenyl)-5-(2-methoxy-4-pyrimidinyl)imidazole;
2-[(4-N,N-dimethyl)aminomethylphenyl]-4-(4-fluorophenyl)-5-(2-phenoxy-4-pyrimidinyl)imidazole;
(+/−)-2-(4-methylsulfinylphenyl]-4-(4-fluorophenyl)-5-(2-phenoxy-4-pyrimidinyl)imidazole;
2-(4-methylthiophenyl]-4-(4-fluorophenyl)-5-(2-phenoxy-4-pyrimidinyl)imidazole; and pharmaceutically acceptable salts thereof.

Compounds of a different scaffold that are also useful in the practice of the present invention include, but are not limited to, compounds of formula: 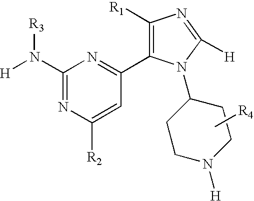
wherein

R₁ is hydrogen, C₁₋₅ alkyl, halogen, C₁₋₅ alkoxy, or arylC₁₋₅ alkyl;

R₂ and R₄ are independently hydrogen, C₁₋₅ alkyl, aryl, arylC₁₋₅ alkyl, heteroaryl, heteroarylC₁₋₅ alkyl, heterocyclic, or heterocyclicC₁₋₅ alkyl; and

R₃ is hydrogen or C₁₋₃ alkyl; or a pharmaceutically-acceptable salt thereof.

Another class of compounds that are useful in the practice of the present invention include, but are not limited to, compounds of formula: 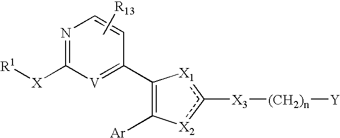
wherein

X is O, CH₂, S or NH, or the moiety X—R¹ is hydrogen;

R¹ is hydrogen, C₁₋₆ alkyl, C₃₋₇ cycloalkyl, aryl, arylC₁₋₆ alkyl, heterocyclyl, heterocyclylC₁₋₆ alkyl, heteroaryl, or heteroarylC₁₋₆ alkyl, any of which, except for hydrogen, can be optionally substituted;

V is CH or N;

Ar is an aryl or heteroaryl ring, either of which can be optionally substituted;

one of X₁ and X₂ is N, and the other is NR¹⁵, wherein R¹⁵ is hydrogen, C₁₋₆ alkyl, or arylC₁₋₆ alkyl;

X₃ is a covalent bond or C(R²)(R³);

R² and R³ independently represent optionally substituted C₁₋₆ alkyl, or R² and R³ together with the carbon atom to which they are attached form an optionally substituted C₃₋₇ cycloalkyl, C₃₋₇ cycloalkenyl, or 5- to 7-membered heterocyclyl ring containing up to three heteroatoms independently selected from N, O, and S;

n is 0,1,2,3, or 4;

Y is NR¹⁰R¹¹, NR¹⁰C(Z)NR¹⁰R¹¹, NR¹⁰COOR¹¹, NR¹⁰SO₂R¹¹, or C(O)NR⁴R⁵;

R⁴ and R⁵ independently represent hydrogen, C₁₋₆ alkyl, C₃₋₇ cycloalkyl, aryl, arylC₁₋₆ alkyl, heteroaryl, heteroarylC₁₋₆ alkyl, heterocyclyl, or heterocyclylC₁₋₆ alkyl, any one of which, except hydrogen, can be optionally substituted, or R⁴ and R⁵ together with the nitrogen atom to which they are attached form a 4- to 10-membered optionally-substituted monocyclic or bicyclic ring;

R¹³ is hydrogen, X—R¹, halogen, optionally-substituted C₁₋₆ alkylsulfinyl, CH₂OR¹⁴, di-C₁₋₆ alkylamino, N(R⁶)C(O)R⁷, N(R⁶)S(O)₂R⁸, or a 5- to 7-membered N-heterocyclyl ring which optionally contains an additional heteroatom selected from O, S, and NR⁹;

R¹⁴ is hydrogen, —C(Z)R¹² or optionally-substituted C₁₋₆ alkyl, optionally-substituted aryl, optionally-substituted arylC₁₋₆ alkyl or S(O)₂R⁸;

R⁶ is hydrogen or C₁₋₆ alkyl;

R⁷ is hydrogen, C₁₋₆ alkyl, C₃₋₇ cycloalkyl, aryl, arylC₁₋₆ alkyl, heteroaryl, heteroarylC₁₋₆ alkyl, heterocyclyl or heterocyclylC₁₋₄ alkyl;

R⁸ is C₁₋₆ alkyl, C₃₋₇ cycloalkyl, aryl, arylC₁₋₆ alkyl, heteroaryl, heteroarylC₁₋₆ alkyl, heterocyclyl or heterocyclylC₁₋₆ alkyl;

R⁹ is hydrogen, cyano, C₁₋₄ alkyl, C₃₋₇ cycloalkyl or aryl;

R¹⁰, R¹¹ and R¹² are independently selected from hydrogen, C₁₋₆ alkyl, C₃₋₇ cycloalkyl, heterocyclyl, heterocyclylC₁₋₆ alkyl, heterocyclylC₂₋₆ alkenyl, aryl, arylC₁₋₆ alkyl, arylC₂₋₆ alkenyl, heteroaryl, heteroarylC₁₋₆ alkyl and heteroarylC₂₋₆ alkenyl, any of which can be optionally substituted; or NR¹⁰R¹¹ can represent a 5- to 7-membered heterocyclyl ring optionally containing an additional heteroatom selected from O, N and S; and

Z is oxygen or sulfur;

or a pharmaceutically-acceptable salt thereof.

Further compounds useful in the practice of the present invention also include, but are not limited to, compounds of formulas: 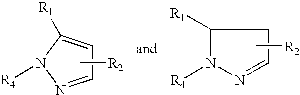
wherein

R₁ is a heteroaryl selected from 4-pyridyl, 4-pyrimidinyl, 4-quinolyl, 6-isoquinolinyl, quinazolin-4-yl, 1-imidazolyl, 1-benzimidazolyl, 4-pyridazinyl, and a 1,2,4-triazin-5-yl ring, which heteroaryl ring is substituted one to three times with Y, NHRₐ, optionally-substituted C₁₋₄ alkyl, halogen, hydroxyl, optionally-substituted C₁₋₄ alkoxy, optionally-substituted C₁₋₄ alkylthio, optionally-substituted C₁₋₄ alkylsulfinyl, CH₂OR₁₂, amino, mono- and di-C₁₋₆ alkyl-substituted amino, N(R₁₀)C(O)R_{b}, N(R₁₀)S(O)₂R_{d}, or an N-heterocyclyl ring which has from 5 to 7 members and optionally contains an additional heteroatom selected from oxygen, sulfur or NR₁₅;

Y is X₁-Rₐ;

X₁ is oxygen or sulfur;

Rₐ is C₁₋₆ alkyl, aryl, arylC₁₋₆ alkyl, heterocyclic, heterocyclylC₁₋₆ alkyl, heteroaryl, or heteroarylC₁₋₆ alkyl, wherein each of these moieties can be optionally substituted;

R_{b} is hydrogen, C₁₋₆ alkyl, C₃₋₇ cycloalkyl, aryl, arylC₁₋₄ alkyl, heteroaryl, heteroarylC₁₋₄ alkyl, heterocyclyl, or heterocyclylC₁₋₄ alkyl;

R_{d} is C₁₋₆ alkyl, C₃₋₇ cycloalkyl, aryl, arylC₁₋₄ alkyl, heteroaryl, heteroarylC₁₋₄ alkyl, heterocyclyl, or heterocyclylC₁₋₄ alkyl;

R₄ is phenyl, naphth-1-yl, naphth-2-yl, a heteroaryl or a fused phenyl-containing ring system, which is optionally substituted by one or two substituents, each of which is independently selected, and which, for a 4-phenyl, 4-naphth-1-yl, 5-naphth-2-yl or 6-naphth-2-yl substituent, is halogen, cyano, nitro, —C(Z)NR₇R₁₇, —C(Z)OR₁₆, —(CR₁₀R₂₀)ᵥCOR₁₂, —SR₅, —SOR₅, —OR₁₂, halo-substituted-C₁₋₄ alkyl, C₁₋₄ alkyl, —ZC(Z)R₁₂, —NR₁₀C(Z)R₁₆, or —(CR₁₀R₂₀)ᵥNR₁₀R₂₀ and which, for other positions of substitution, is halogen, cyano, nitro, phenyl, —C(Z)NR₁₃R₁₄, —C(Z)OR_{f}, —(CR₁₀R₂₀)_{m″}COR_{f}, —S(O)ₘR_{f}, —OR_{f}, halo-substituted C₁₋₄ alkyl, C₁₋₁₀ alkyl, —ZC(Z)R_{f}, optionally-substituted phenyl, —(CR₁₀R₂₀)_{m″}NR₁₀C(Z)R_{f}, —NR₁₀S(O)_{m′}R₈, —NR₁₀S(O)_{m′}NR₇R₁₇, —ZC(Z)R₁₂, or —(CR₁₀R₂₀)_{m″}NR₁₃R₁₄;

R_{f} is heterocyclyl, heterocyclylC₁₋₁₀ alkyl or R₈;

v is 0, 1, or 2;

m is 0, 1, or 2;

m′ is 1 or 2;

m″ is 0, 1, 2, 3, 4, or 5;

R₂ hydrogen, —(CR₁₀R₂₃)ₙOR₉, heterocylyl, heterocyclylC₁₋₁₀ alkyl, C₁₋₁₀ alkyl, halo-substituted C₁₋₁₀ alkyl, C₂₋₁₀ alkenyl, C₂₋₁₀ alkynyl, C₃₋₇ cycloalkyl, C₃₋₇ cycloalkylC₁₋₁₀ alkyl, C₅₋₇ cycloalkenyl, C₅₋₇cycloalkenylC₁₋₁₀ alkyl, aryl, arylC₁₋₁₀ alkyl, heteroaryl, heteroarylC₁₋₁₀ alkyl, (CR₁₀R₂₃)ₙOR₁₁, (CR₁₀R₂₃)ₙS(O)ₘR₁₈, (CR₁₀R₂₃)ₙNHS(O)₂R₁₈, (CR₁₀R₂₃)ₙNR₁₃R₁₄, (CR₁₀R₂₃)ₙNO₂, (CR₁₀R₂₃)ₙCN, (CR₁₀R₂₃)ₙS(O)_{m′}NR₁₃R₁₄, (CR₁₀R₂₃)ₙC(Z)R₁₁, (CR₁₀R₂₃)ₙOC(Z)R₁₁, (CR₁₀R₂₃)ₙC(Z)OR₁₁, (CR₁₀R₂₃)ₙC(Z)NR₁₃R₁₄, (CR₁₀R₂₃)ₙC(Z)NR₁₁OR₉, (CR₁₀R₂₃)ₙNR₁₀C(Z)R₁₁, (CR₁₀R₂₃)ₙNR₁₀C(Z)NR₁₃R₁₄, (CR₁₀R₂₃)ₙN(OR₆)C(Z)NR₁₃R₁₄, (CR₁₀R₂₃)ₙN(OR₆)C(Z)R₁₁, (CR₁₀R₂₃)ₙC(═NOR₆)R₁₁, (CR₁₀R₂₃)ₙNR₁₀C(═NR₁₉)NR₁₃R₁₄, (CR₁₀R₂₃)ₙOC(Z)NR₁₃R₁₄, (CR₁₀R₂₃)ₙNR₁₀C(Z)NR₁₃R₁₄, (CR₁₀R₂₃)ₙNR₁₀C(Z)OR₁₀, 5-(R₁₈)-1,2,4-oxadiazol-3-yl or 4-(R₁₂)-5-(R₁₈R₁₉)-4,5-dihydro-1,2,4-oxadiazol-3-yl; wherein the aryl, arylalkyl, heteroaryl, heteroaryl alkyl, cycloalkyl, cycloalkyl alkyl, heterocyclic and heterocyclic alkyl groups can be optionally substituted;

n is 0, or an integer having a value of 1 to 10;

Z is oxygen or sulfur;

R₅ is hydrogen, C₁₋₄ alkyl, C₂₋₄ alkenyl, C₂₋₄ alkynyl or NR₇R₁₇, excluding the moieties —SR₅ being —SNR₇R₁₇ and —S(O)R₅ being —SOH;

R₆ is hydrogen, a pharmaceutically-acceptable cation, C₁₋₁₀ alkyl, C₃₋₇ cycloalkyl, aryl, arylC₁₋₄ alkyl, heteroaryl, heteroarylC₁₋₄ alkyl, heterocyclyl, aroyl, or C₁₋₁₀ alkanoyl;

R₇ and R₁₇ are each independently selected from hydrogen or C₁₋₄ alkyl, or R₇ and R₁₇ together with the nitrogen to which they are attached form a heterocyclic ring of 5 to 7 members which ring optionally contains an additional heteroatom selected from oxygen, sulfur or NR₁₅;

R₈ is C₁₋₁₀ alkyl, halo-substituted C₁₋₁₀ alkyl, C₂₋₁₀ alkenyl, C₂₋₁₀ alkynyl, C₃₋₇ cycloalkyl, C₅₋₇ cycloalkenyl, aryl, arylC₁₋₁₀ alkyl, heteroaryl, heteroarylC₁₋₁₀ alkyl, (CR₁₀R₂₀)ₙOR₁₁, (CR₁₀R₂₀)ₙS(O)ₘR₁₈, (CR₁₀R₂₀)ₙNHS(O)₂R₁₈, or (CR₁₀R₂₀)ₙNR₁₃R₁₄, wherein the aryl, arylalkyl, heteroaryl, and heteroaryl alkyl can be optionally substituted;

R₉ is hydrogen, —C(Z)R₁₁, optionally-substituted C₁₋₁₀ alkyl, S(O)₂R₁₈, optionally-substituted aryl or optionally-substituted arylC₁₋₄ alkyl;

R₁₀ and R₂₀ are each independently selected from hydrogen or C₁₋₄ alkyl;

R₁₁ is hydrogen, C₁₋₁₀ alkyl, C₃₋₇ cycloalkyl, heterocyclyl, heterocyclylC₁₋₁₀ alkyl, aryl, arylC₁₋₁₀ alkyl, heteroaryl or heteroarylC₁₋₁₀ alkyl, wherein the aryl, arylalkyl, heteroaryl, heteroaryl alkyl, heterocyclyl or heterocyclylalkyl can be optionally substituted;

R₁₂ is hydrogen or R₁₆;

R₁₃ and R₁₄ are each independently selected from hydrogen or optionally-substituted C₁₋₄ alkyl, optionally-substituted aryl or optionally-substituted arylC₁₋₄ alkyl, or together with the nitrogen to which they are attached form a heterocyclic ring of 5 to 7 members which ring optionally contains an additional heteroatom selected from oxygen, sulfur or NR₉;

R₁₅ is hydrogen, C₁₋₄ alkyl or C(Z)—C₁₋₄ alkyl;

R₁₆ is C₁₋₄ alkyl, halo-substituted C₁₋₄ alkyl, or C₃₋₇ cycloalkyl;

R₁₈ is C₁₋₁₀ alkyl, C₃₋₇ cycloalkyl, heterocyclyl, aryl, arylC₁₋₁₀ alkyl, heterocyclyl, heterocyclylC₁₋₁₀ alkyl, heteroaryl or heteroarylC₁₋₁₀ alkyl, wherein the aryl, arylalkyl, heteroaryl, heteroaryl alkyl, heterocyclyl or heterocyclylalkyl can be optionally substituted;

R₁₉ is hydrogen, cyano, C₁₋₄ alkyl, C₃₋₇ cycloalkyl or aryl; and

R₂₃ is hydrogen, C₁₋₆ alkyl, C₃₋₇ cycloalkyl, aryl, arylC₁₋₄ alkyl, heteroaryl, heteroarylC₁₋₄ alkyl, heterocyclyl, or heterocyclylC₁₋₄ alkyl, all of which can be optionally substituted;

or a pharmaceutically-acceptable salt thereof.

Exemplary compounds of this class include but are not limited to:
4-[1-(4-fluorophenyl)-3-phenyl-1H-pyrazol-5-yl]pyridine
4-[4-bromo-1-(4-fluorophenyl)-3-phenyl-1H-pyrazol-5-yl]pyridine
4-[1-(4-fluorophenyl)-3-[4-(methylthio)phenyl]-1H-pyrazol-5-yl]pyridine
4-[1-(4-fluorophenyl)-3-[4-(methylsulfonyl)phenyl]-1H-pyrazol-5-yl]pyridine 4-[1-(4-fluorophenyl)-3-[4-(methylsulfinyl)phenyl]-1 H-pyrazol-5-yl]pyridine;
4-[1-(4-fluorophenyl)-4,5-dihydro-3-phenyl-1H-pyrazol-5-yl]pyridine
4-[1-(4-fluorophenyl)-4,5-dihydro-3-[4-(methylthio)phenyl]-1H-pyrazol-5-yl]pyridine
   and pharmaceutically acceptable salts thereof.

Similar compounds that are also useful in the practice of the present invention include, but are not limited to, compounds of formulas: 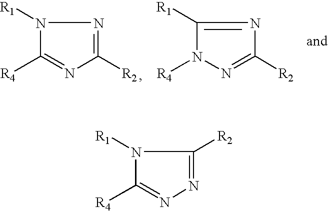
wherein

R₁ is 4-pyridyl or 4-pyrimidinyl ring, which ring is optionally substituted one or more times with Y, C₁₋₄ alkyl, halogen, hydroxyl, C₁₋₄ alkoxy, C₁₋₄ alkylthio, C₁₋₄ alkylsulfinyl, CH₂OR₁₂, amino, mono- and di-C₁₋₆ alkyl-substituted amino, N(R₁₀)C(O)R_{b}, or an N-heterocyclyl ring which has from 5 to 7 members and optionally contains an additional heteroatom selected from oxygen, sulfur or NR₁₅;

Y is X₁—Rₐ;

X₁ is oxygen, sulfur, or NH;

Rₐ is C₁₋₆ alkyl, aryl, arylC₁₋₆ alkyl, heterocyclic, heterocyclylC₁₋₆ alkyl, heteroaryl, or heteroarylC₁₋₆ alkyl, wherein each of these moieties can be optionally substituted;

R_{b} is hydrogen, C₁₋₆ alkyl, C₃₋₇ cycloalkyl, aryl, arylC₁₋₄ alkyl, heteroaryl, heteroarylC₁₋₄ alkyl, heterocyclyl, or heterocyclylC₁₋₄ alkyl, wherein each of these moieties can be optionally substituted;

R₄ is phenyl, naphth-1-yl, naphth-2-yl, or a heteroaryl, which is optionally substituted by one or two substituents, each of which is independently selected, and which, for a 4-phenyl, 4-naphth-1-yl, 5-naphth-2-yl or 6-naphth-2-yl substituent, is halogen, cyano, nitro, —C(Z)NR₇R₁₇, —C(Z)OR₁₆, —(CR₁₀R₂₀)ᵥCOR₁₂, —SR₅, —SOR₅, —OR₁₂, halo-substituted-C₁₋₄ alkyl, C₁₋₄ alkyl, —ZC(Z)R₁₂, —NR₁₀C(Z)R₁₆, or —(CR₁₀R₂₀)ᵥNR₁₀R₂₀ and which, for other positions of substitution, is halogen, cyano, —C(Z)NR₁₃R₁₄, —C(Z)OR_{f}, —(CR₁₀R₂₀)_{m″}COR_{f}, —S(O)ₘR_{f}, —OR_{f}, halo-substituted C₁₋₄ alkyl, C₁₋₄ alkyl, —ZC(Z)R_{f}, —(CR₁₀R₂₀)_{m″}NR₁₀C(Z)R_{f}, —NR₁₀S(O)_{m′}R₈, —NR₁₀S(O)_{m″}NR₇R₁₇, or —(CR₁₀R₂₀)_{m″}NR₁₃R₁₄;

R_{f} is heterocyclyl, heterocyclylC₁₋₁₀ alkyl or R₈;

v is 0, 1, or 2;

m is 0, 1, or 2;

m′ is 1 or 2;

m″ is 0,1,2,3,4, or 5;

R₂ hydrogen, C(HOURS)(A)(R₂₂), —(CR₁₀R₂₃)ₙOR₉, heterocylyl, heterocyclylC₁₋₁₀ alkyl, C₁₋₁₀ alkyl, halo-substituted C₁₋₁₀ alkyl, C₂₋₁₀ alkenyl, C₂₋₁₀ alkynyl, C₃₋₇ cycloalkyl, C₃₋₇ cycloalkylC₁₋₁₀ alkyl, C₅₋₇ cycloalkenyl, C₅₋₇cycloalkenylC₁₋₁₀ alkyl, aryl, arylC₁₋₁₀ alkyl, heteroaryl, heteroarylC₁₋₁₀ alkyl, (CR₁₀R₂₃)ₙOR₁₁, (CR₁₀R₂₃)ₙS(O)ₘR₁₈, (CR₁₀R₂₃)ₙNHS(O)₂R₁₈, (CR₁₀R₂₃)ₙNR₁₃R₁₄, (CR₁₀R₂₃)ₙNO₂, (CR₁₀R₂₃)ₙCN, (CR₁₀R₂₃)ₙS(O)_{m′}NR₁₃R₁₄, (CR₁₀R₂₃)ₙC(Z)R₁₁, (CR₁₀R₂₃)ₙOC(Z)R₁₁, (CR₁₀R₂₃)ₙC(Z)OR₁₁, (CR₁₀R₂₃)ₙC(Z)NR₁₃R₁₄, (CR₁₀R₂₃)ₙC(Z)NR₁₁OR₉, (CR₁₀R₂₃)ₙNR₁₀C(Z)R₁₁, (CR₁₀R₂₃)ₙNR₁₀C(Z)NR₁₃R₁₄, (CR₁₀R₂₃)ₙN(OR₆)C(Z)NR₁₃R₁₄, (CR₁₀R₂₃)ₙN(OR₆)C(Z)R₁₁, (CR₁₀R₂₃)ₙC(═NOR₆)R₁₁, (CR₁₀R₂₃)ₙNR₁₀C(═NR₁₉)NR₁₃R₁₄, (CR₁₀R₂₃)ₙOC(Z)NR₁₃R₁₄, (CR₁₀R₂₃)ₙNR₁₀C(Z)NR₁₃R₁₄, (CR₁₀R₂₃)ₙNR₁₀C(Z)OR₁₀, 5-(R₁₈)-1,2,4-oxadiazol-3-yl or 4-(R₁₂)-5-(R₁₈R₁₉)-4,5-dihydro-1,2,4-oxadiazol-3-yl; wherein the aryl, arylalkyl, heteroaryl, heteroaryl alkyl, cycloalkyl, cycloalkyl alkyl, heterocyclic and heterocyclic alkyl groups can be optionally substituted;

A is an optionally-substituted aryl, heterocyclyl or heteroaryl ring, or A is a substituted C₁₋₁₀ alkyl;

n is 0, or an integer having a value of 1 to 10;

Z is oxygen or sulfur;

R₅ is hydrogen, C₁₋₄ alkyl, C₂₋₄ alkenyl, C₂₋₄ alkynyl or NR₇R₁₇, excluding the moieties —SR₅ being —SNR₇R₁₇ and —S(O)R₅ being —SOH;

R₆ is hydrogen, a pharmaceutically-acceptable cation, C₁₋₁₀ alkyl, C₃₋₇ cycloalkyl, aryl, arylC₁₋₄ alkyl, heteroaryl, heteroarylC₁₋₄ alkyl, heterocyclyl, aroyl, or C₁₋₁₀ alkanoyl;

R₇ and R₁₇ are each independently selected from hydrogen or C₁₋₄ alkyl, or R₇ and R₁₇ together with the nitrogen to which they are attached form a heterocyclic ring of 5 to 7 members which ring optionally contains an additional heteroatom selected from oxygen, sulfur or NR₁₅;

R₈ is C₁₋₁₀ alkyl, halo-substituted C₁₋₁₀ alkyl, C₂₋₁₀ alkenyl, C₂₋₁₀ alkynyl, C₃₋₇ cycloalkyl, C₅₋₇ cycloalkenyl, aryl, arylC₁₋₁₀ alkyl, heteroaryl, heteroarylC₁₋₁₀ alkyl, (CR₁₀R₂₀)ₙOR₁₁, (CR₁₀R₂₀)ₙS(O)ₘR₁₈, (CR₁₀R₂₀)ₙNHS(O)₂R₁₈, or (CR₁₀R₂₀)ₙNR₁₃R₁₄, wherein the aryl, arylalkyl, heteroaryl, and heteroaryl alkyl can be optionally substituted;

R₉ is hydrogen, —C(Z)R₁₁, optionally-substituted C₁₋₁₀ alkyl, S(O)₂R₁₈, optionally-substituted aryl or optionally-substituted arylC₁₋₄ alkyl;

R₁₀ and R₂₀ are each independently selected from hydrogen or C₁₋₄ alkyl;

R₁₁ is hydrogen, C₁₋₁₀ alkyl, C₃₋₇ cycloalkyl, heterocyclyl, heterocyclylC₁₋₁₀ alkyl, aryl, arylC₁₋₁₀ alkyl, heteroaryl or heteroarylC₁₋₁₀ alkyl, wherein the aryl, arylalkyl, heteroaryl, heteroaryl alkyl, heterocyclyl or heterocyclylalkyl can be optionally substituted;

R₁₂ is hydrogen or R₁₆;

R₁₃ and R₁₄ are each independently selected from hydrogen or optionally-substituted C₁₋₄ alkyl, optionally-substituted aryl or optionally-substituted arylC₁₋₄ alkyl, or together with the nitrogen to which they are attached form a heterocyclic ring of 5 to 7 members which ring optionally contains an additional heteroatom selected from oxygen, sulfur or NR₉;

R₁₅ is R₁₀ or C(Z)C₁₋₄ alkyl;

R₁₆ is C₁₋₄ alkyl, halo-substituted C₁₋₄ alkyl, or C₃₋₇ cycloalkyl;

R₁₈ is C₁₋₁₀ alkyl, C₃₋₇ cycloalkyl, heterocyclyl, aryl, arylC₁₋₁₀ alkyl, heterocyclyl, heterocyclylC₁₋₁₀ alkyl, heteroaryl or heteroarylC₁₋₁₀ alkyl;

R¹⁹ is hydrogen, cyano, C₁₋₄ alkyl, C₃₋₇ cycloalkyl or aryl; and

R₂₃ is hydrogen, C₁₋₆ alkyl, C₃₋₇ cycloalkyl, aryl, arylC₁₋₄ alkyl, heteroaryl, heteroarylC₁₋₄ alkyl, heterocyclyl, or heterocyclylC₁₋₄ alkyl, all of which can be optionally substituted;

or a pharmaceutically-acceptable salt thereof.

Examples of this class of compounds include but are not limited to:
1-(pyrid-4-yl)-3-phenyl-5-(4-fluorophenyl)-1,2,4-triazole;
1-(6-aminopyrimidin-4-yl)-3-phenyl-5-(4-fluorophenyl)-1,2,4-triazole;
1-[4-(6,7-dimethoxyquinazoline)]-3-phenyl-5-(4-fluorophenyl)-1,2,4-triazole;
1-(4-fluorophenyl)-3-phenyl-5-(2-aminopyrimidin-4-yl)-1,2,4-triazole;
3-(4-fluorophenyl)-4-(2-aminopyrimidin-4-yl)-5-phenyl-1,2,4-triazole;
   and pharmaceutically acceptable salts thereof.

Yet another class of compounds that are useful in the practice of the present invention includes compounds of formula: 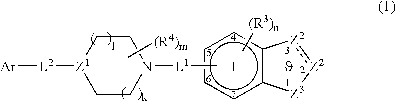
and the pharmaceutically acceptable salts thereof, or a pharmaceutical composition thereof, wherein 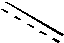
represents a single or double bond;

one Z² is CA or CR⁸A and the other is CR¹, CR¹₂, NR⁶ or N wherein each R¹, R⁶ and R⁸ is independently hydrogen or noninterfering substituent;

A is —CO(X)ⱼY wherein Y is COR² or an isostere thereof and R² is hydrogen or a noninterfering substituent, X is a spacer preferably of 2-6 Å, and j is 0 or 1;

Z³ is NR⁷ or O;

each R³ is independently a noninterfering substituent;

n is 0-3;

each of L¹ and L² is a linker;

each R⁴ is independently a noninterfering substituent;

m is 0-4;

Z¹ is CR⁵ or N wherein R⁵ is hydrogen or a noninterfering substituent;

each of l and k is an integer from 0-2 wherein the sum of l and k is 0-3;

Ar is an aryl group substituted with 0-5 noninterfering substituents, wherein two noninterfering substituents can form a fused ring; and the distance between the atom of Ar linked to L² and the center of the α ring is preferably less than 24 Å. As provided above, certain positions of the molecule are described as permitting “noninterfering substituents.” This terminology is where there is a wide variety of substituents possible in these positions as determined by those skilled in the art with the proviso however that the substituents must not impart a detrimental effect on the essential activity or application of the molecule when taken as a whole.

Exemplary Compounds of this formula include but are not limited to those in the following Table A

TABLE A

| Compd.# | STRUCTURE |
|---|---|
| 1 | 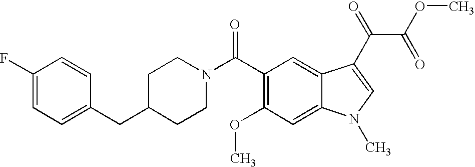 |
| 2 | 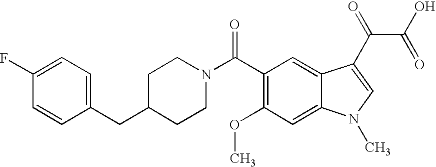 |
| 3 | 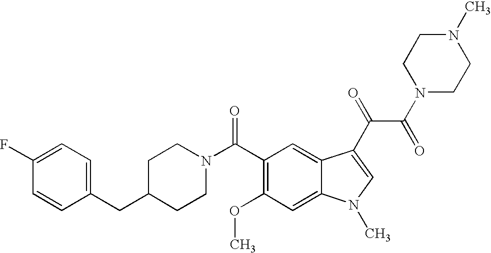 |
| 4 | 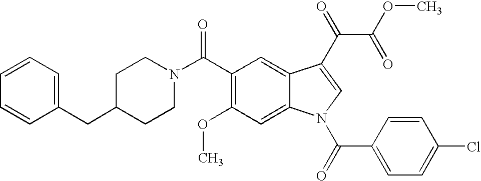 |
| 5 | 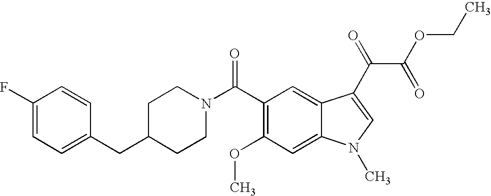 |
| 6 | 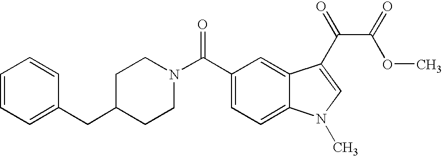 |
| 7 | 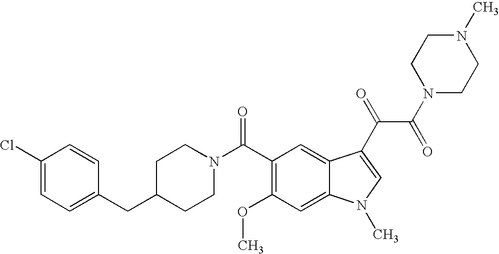 |
| 8 | 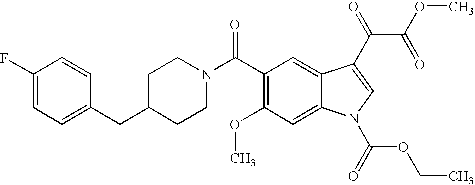 |
| 9 | 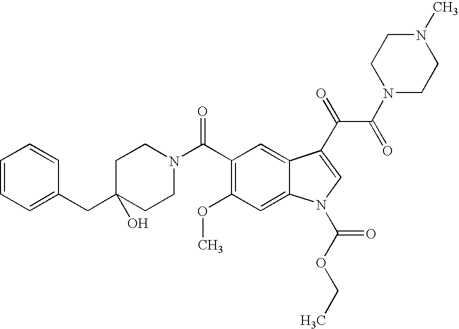 |
| 10 | 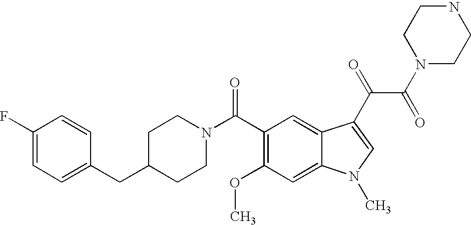 |
| 11 | 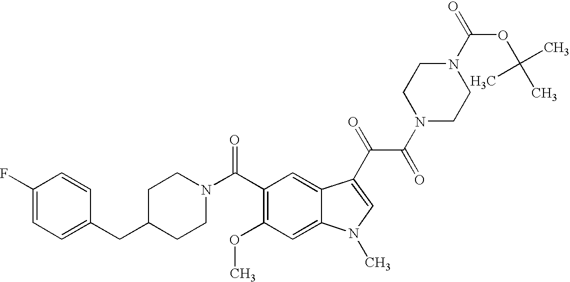 |
| 12 | 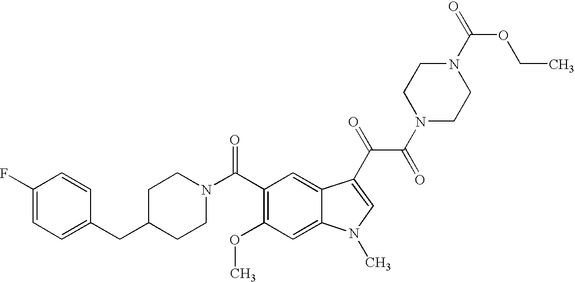 |
| 13 | 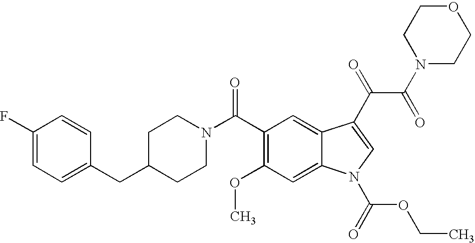 |
| 14 | 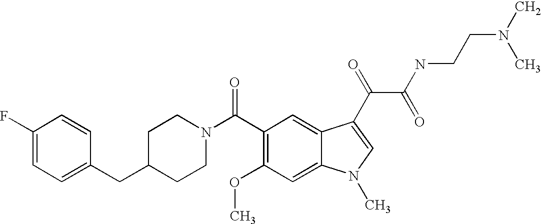 |
| 15 | 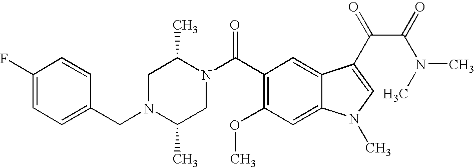 |
| 16 | 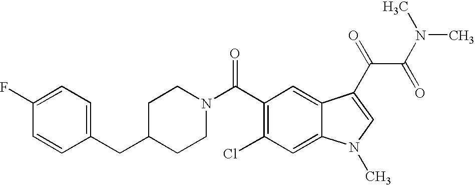 |
| 17 | 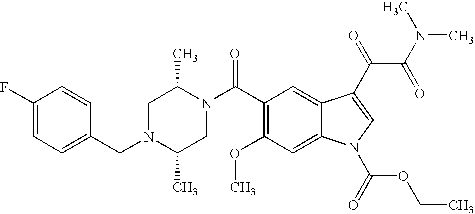 |
| 18 | 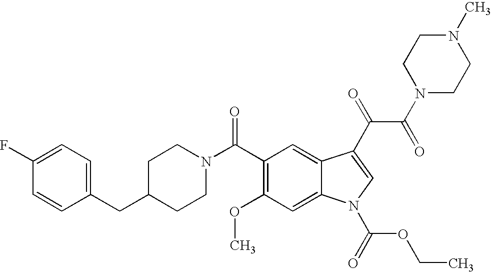 |
| 19 | 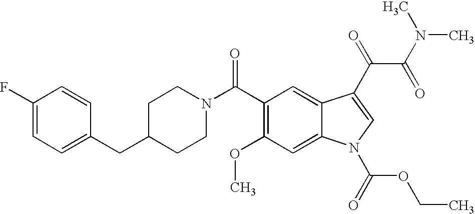 |
| 20 | 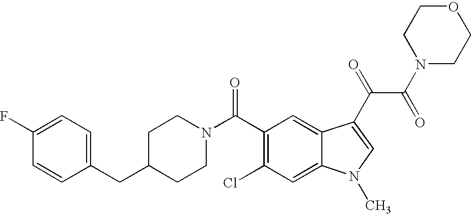 |
| 21 | 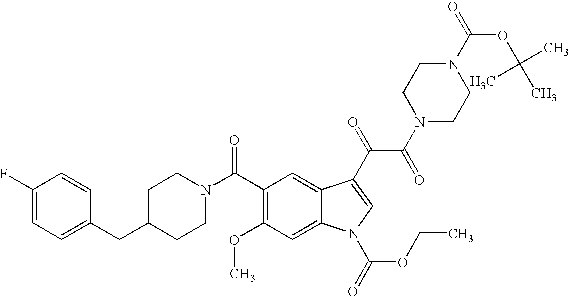 |
| 22 | 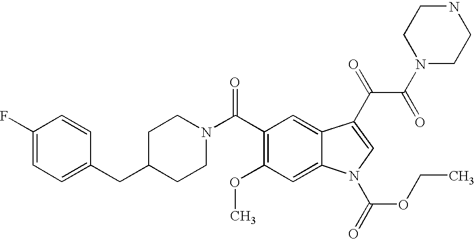 |
| 23 | 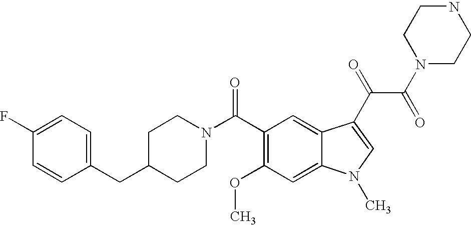 |
| 24 | 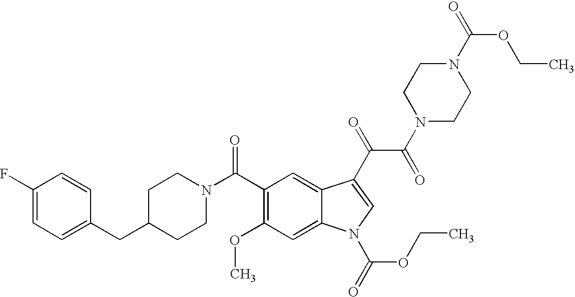 |
| 25 | 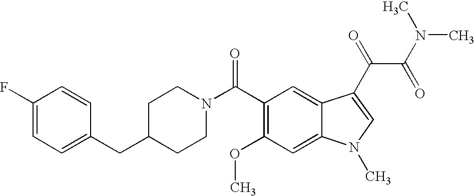 |
| 26 | 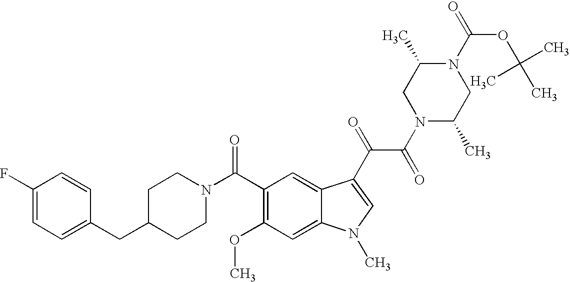 |
| 27 | 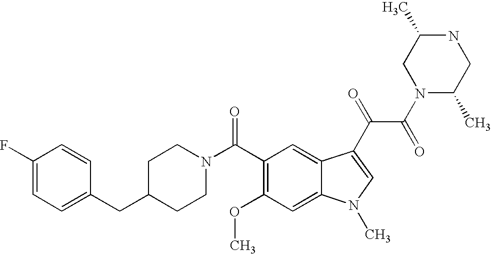 |
| 28 | 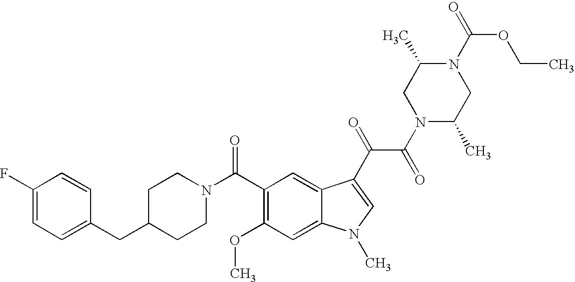 |
| 29 | 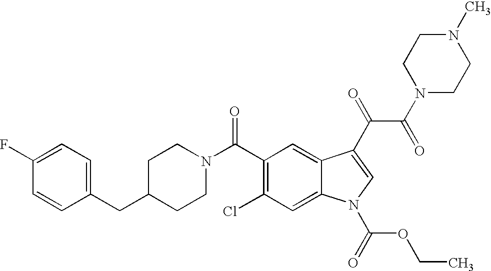 |
| 30 | 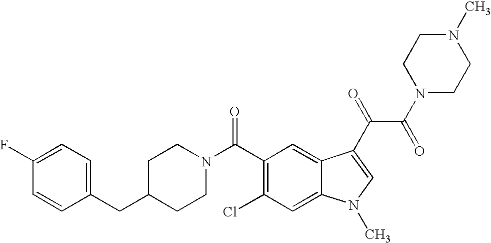 |
| 31 | 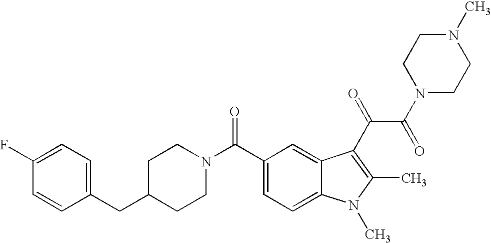 |
| 32 | 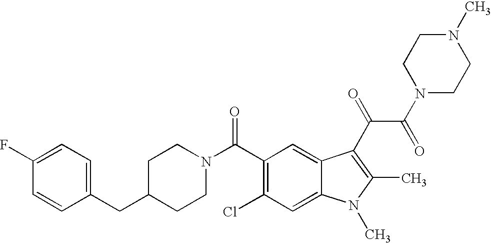 |
| 33 | 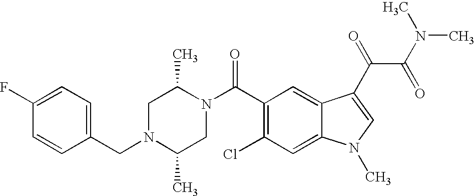 |
| 34 | 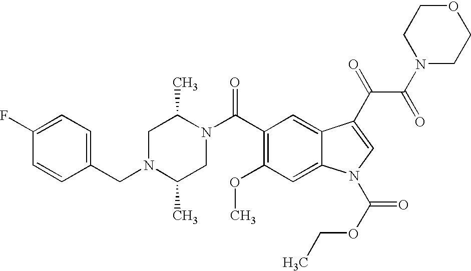 |
| 35 | 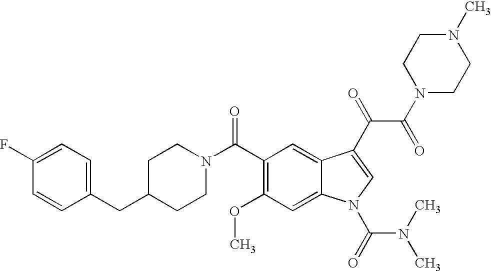 |
| 36 | 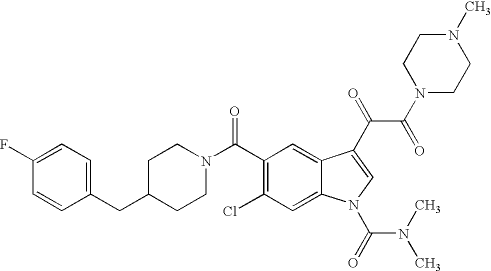 |
| 37 | 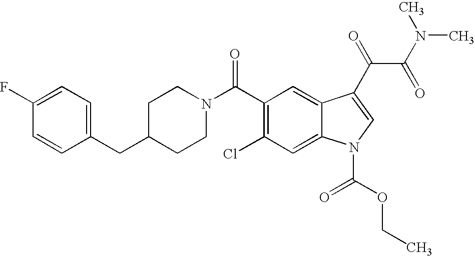 |
| 38 | 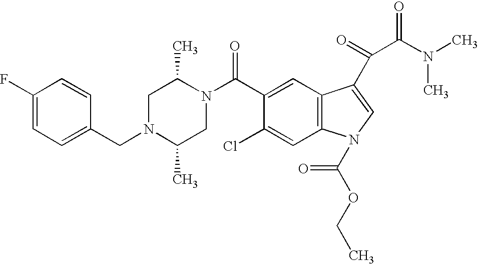 |
| 39 | 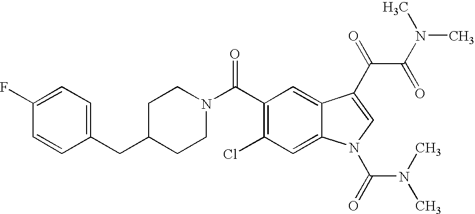 |
| 40 | 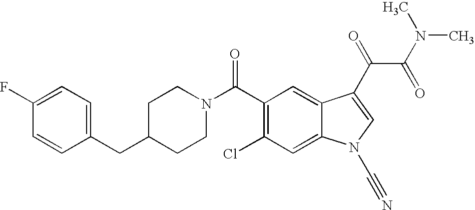 |
| 41 | 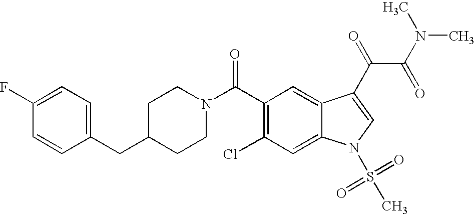 |
| 42 | 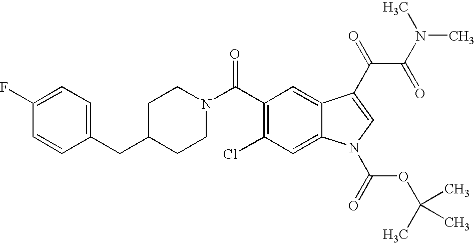 |
| 43 | 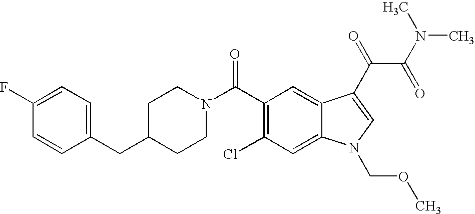 |
| 44 | 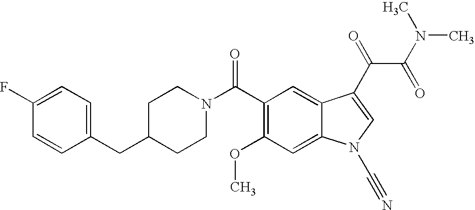 |
| 45 | 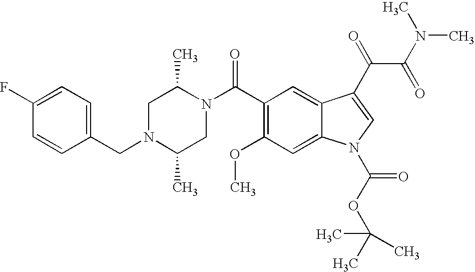 |
| 46 | 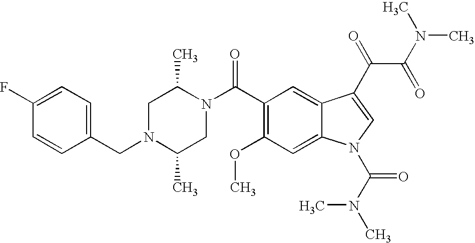 |
| 47 | 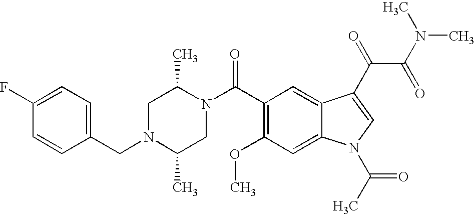 |
| 48 | 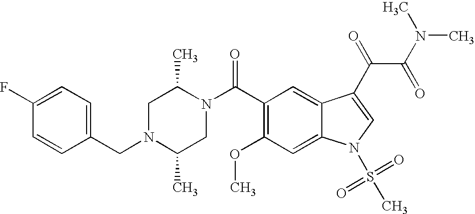 |
| 49 | 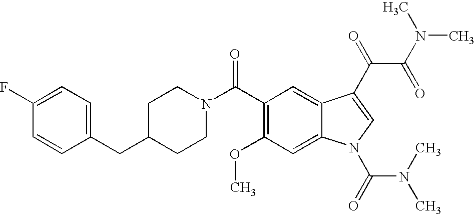 |
| 50 | 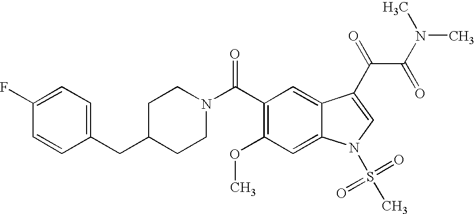 |
| 51 | 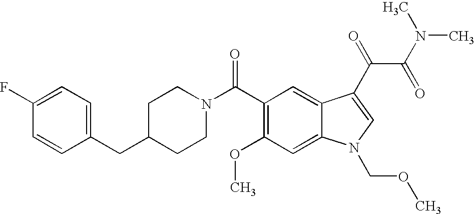 |
| 52 | 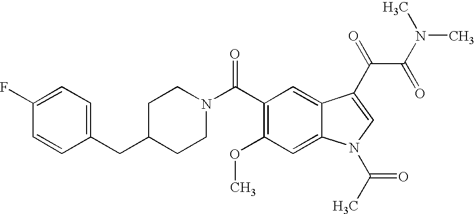 |
| 53 | 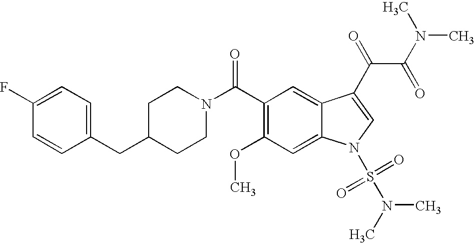 |
| 54 | 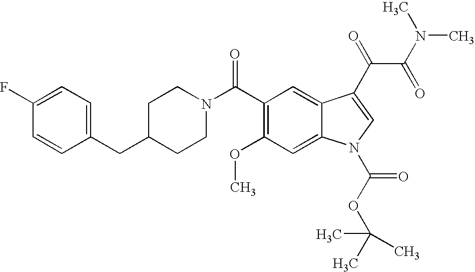 |
| 55 | 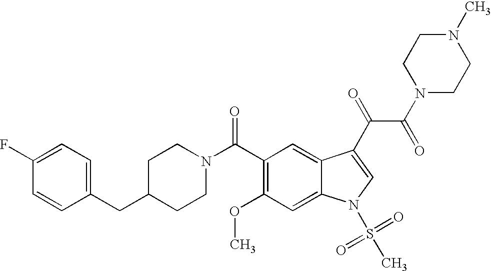 |
| 56 | 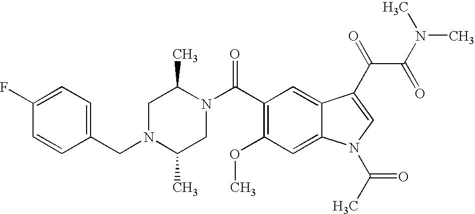 |
| 57 | 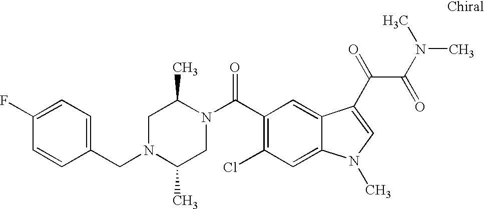 |
| 58 | 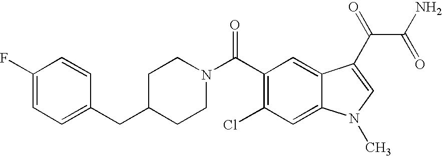 |
| 59 | 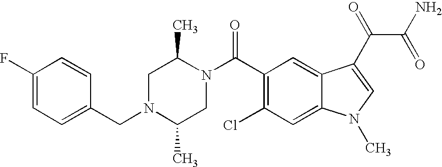 |
| 60 | 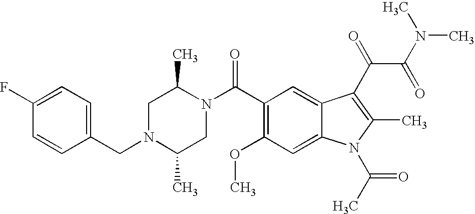 |
| 61 | 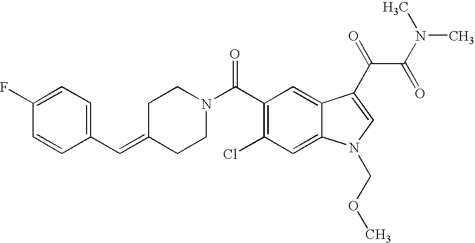 |
| 62 | 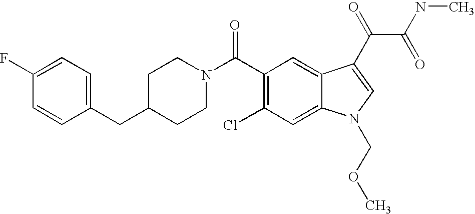 |
| 63 | 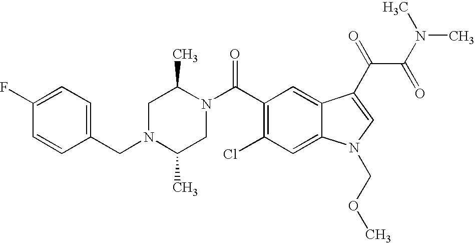 |
| 64 | 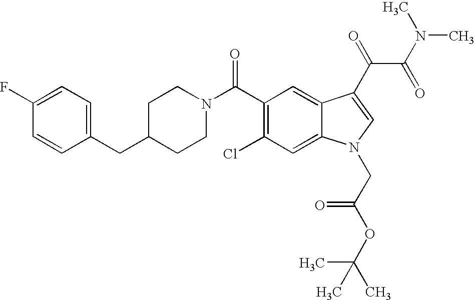 |
| 65 | 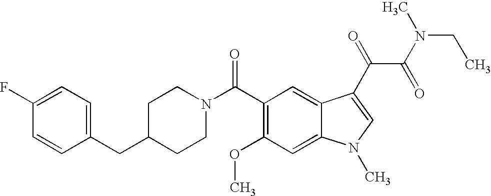 |
| 66 | 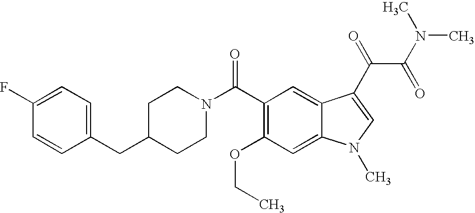 |
| 67 | 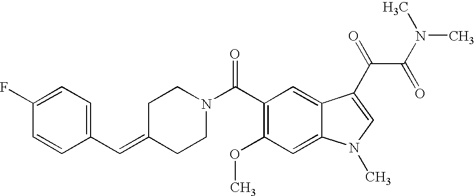 |
| 68 | 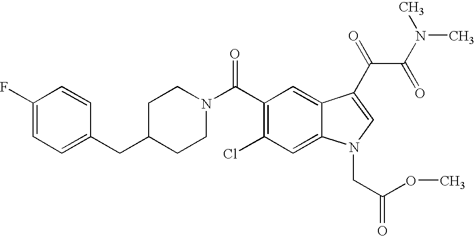 |
| 69 | 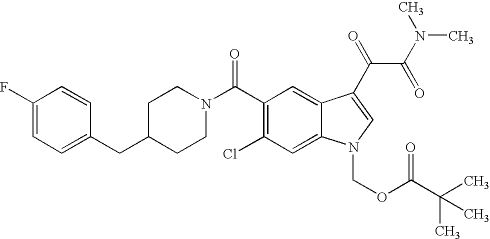 |
| 70 | 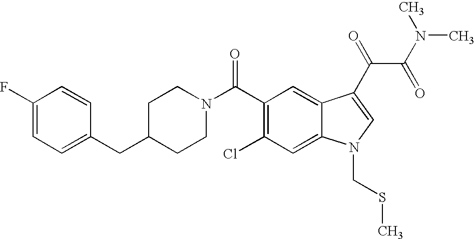 |
| 71 | 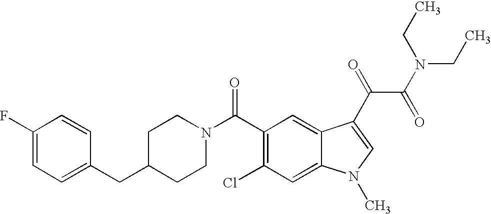 |
| 72 | 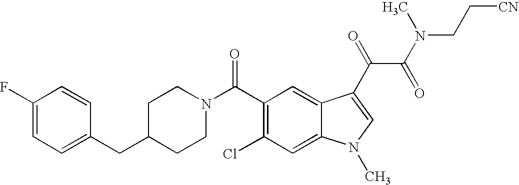 |
| 73 | 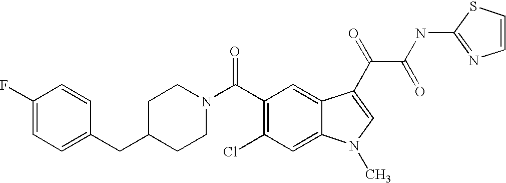 |
| 74 | 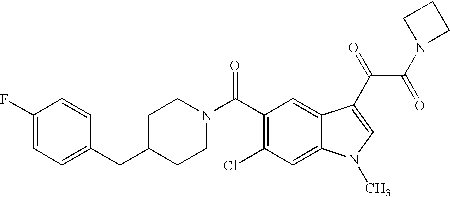 |
| 75 | 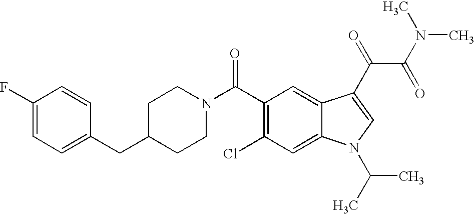 |
| 76 | 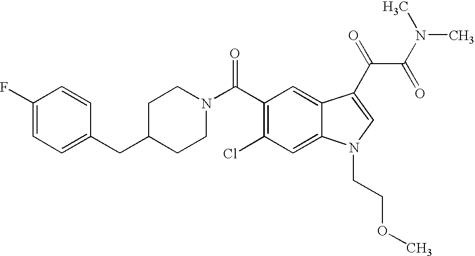 |
| 77 | 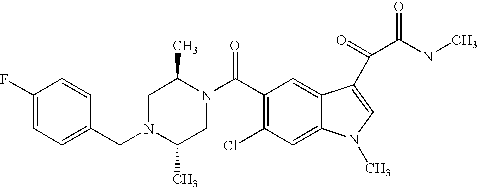 |
| 78 | 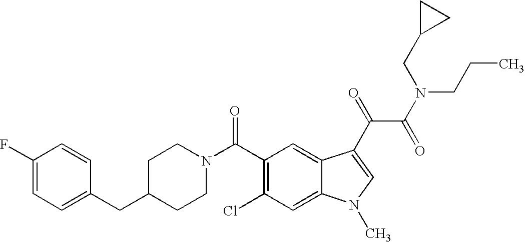 |
| 79 | 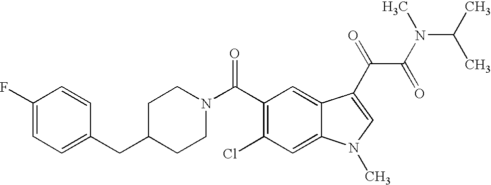 |
| 80 | 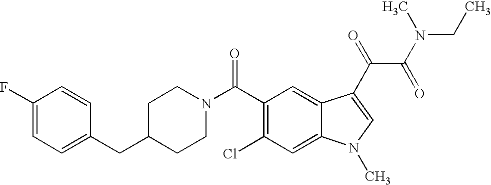 |
| 81 | 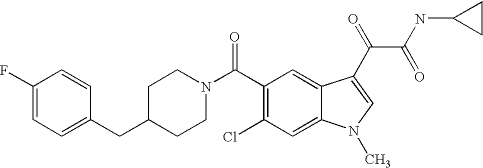 |
| 82 | 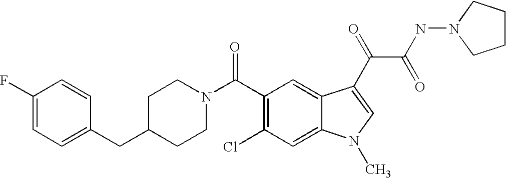 |
| 83 | 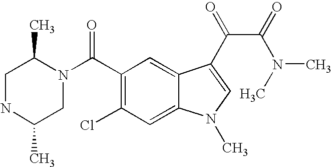 |
| 84 | 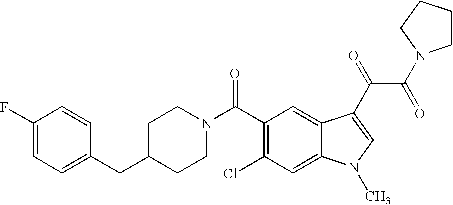 |
| 85 | 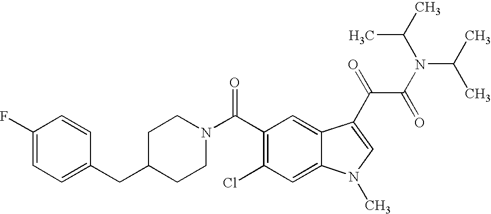 |
| 86 | 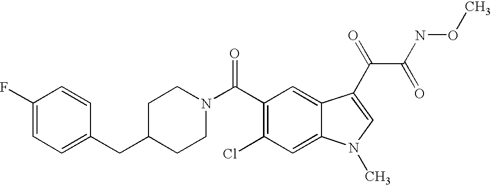 |
| 87 | 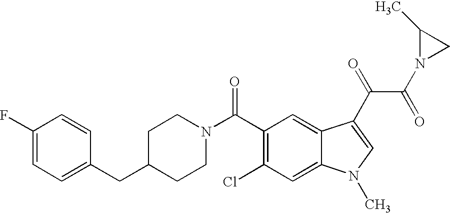 |
| 88 | 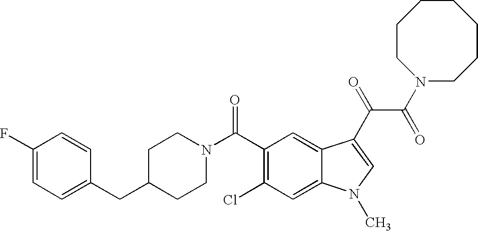 |
| 89 | 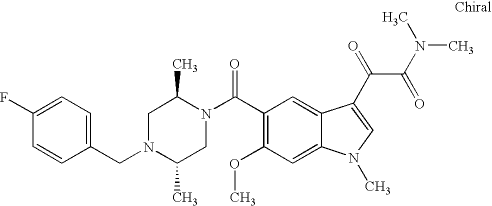 |
| 90 | 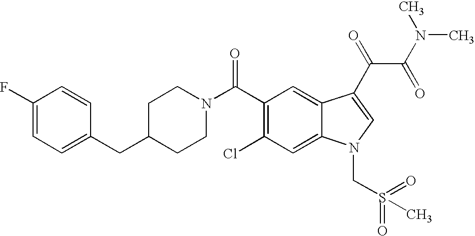 |
| 91 | 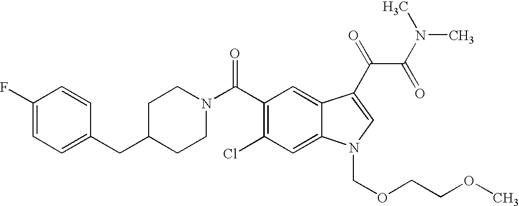 |
| 92 | 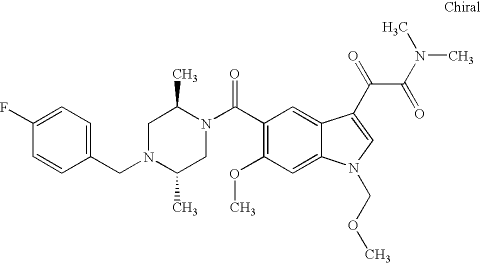 |
| 93 | 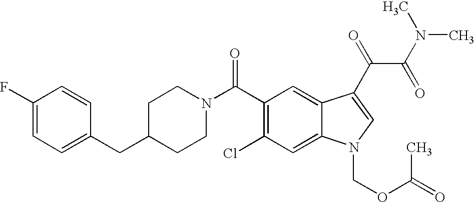 |
| 94 | 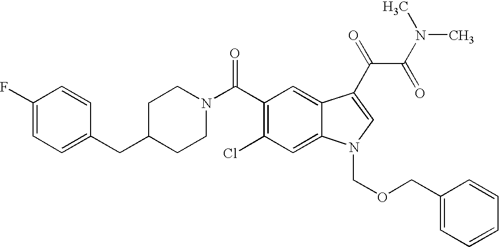 |
| 95 | 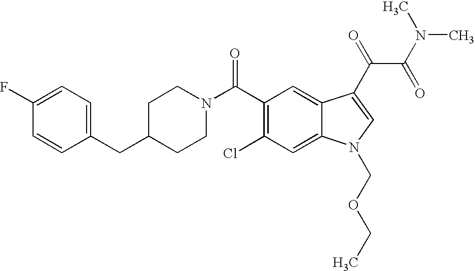 |
| 96 | 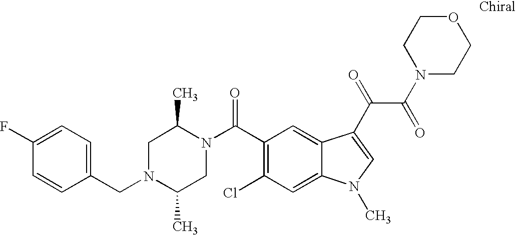 |
| 97 | 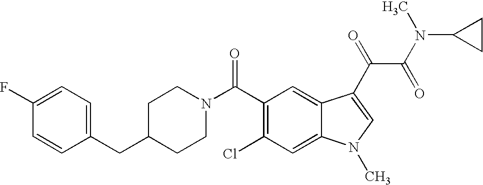 |
| 98 | 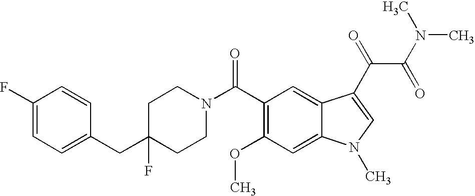 |
| 99 | 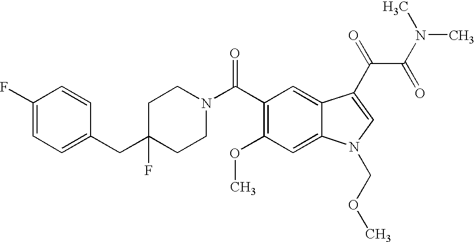 |
| 100 | 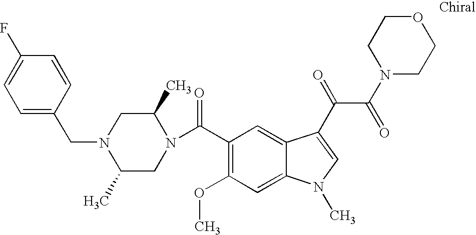 |
| 101 | 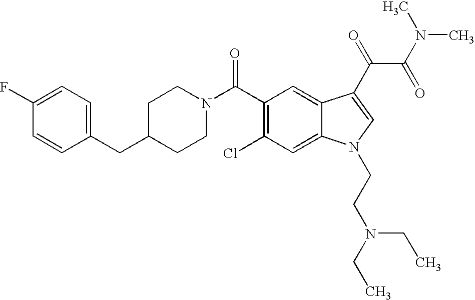 |
| 102 | 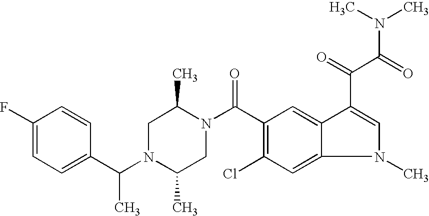 |
| 103 | 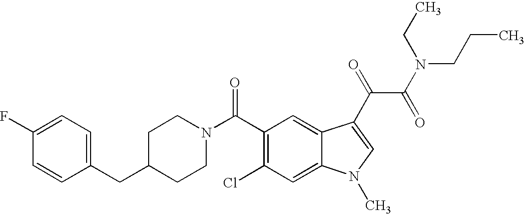 |
| 104 | 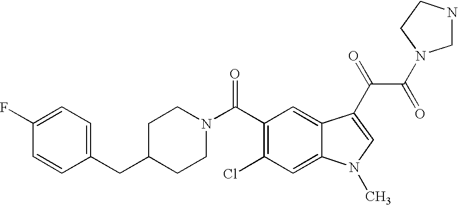 |
| 105 | 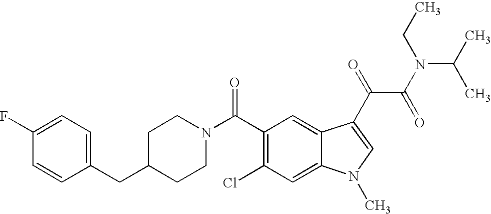 |
| 106 | 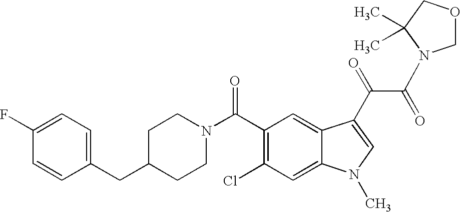 |
| 107 | 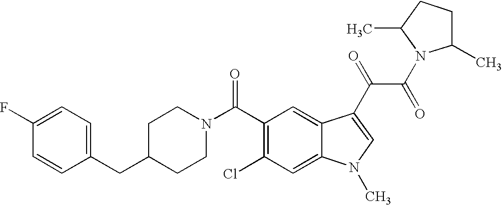 |
| 108 | 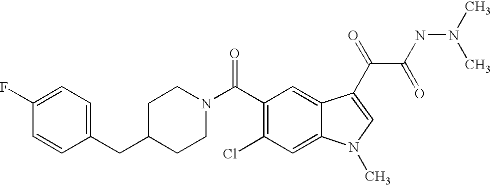 |
| 109 | 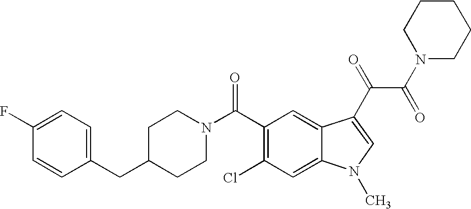 |
| 110 | 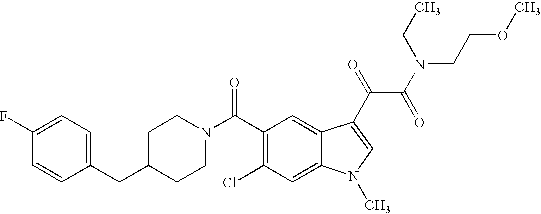 |
| 111 | 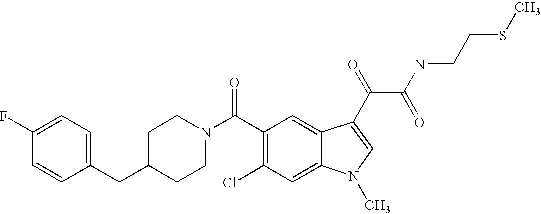 |
| 112 | 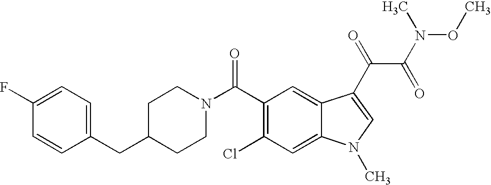 |
| 113 | 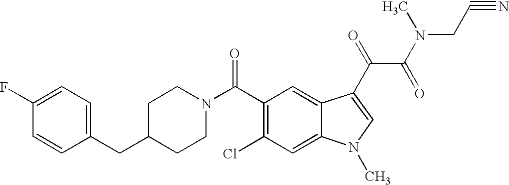 |
| 114 | 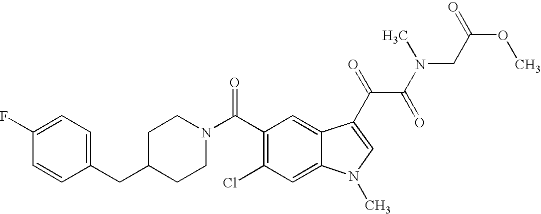 |
| 115 | 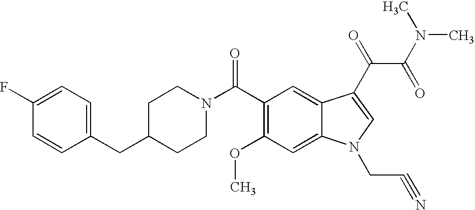 |
| 116 | 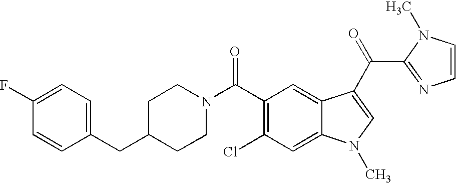 |
| 117 | 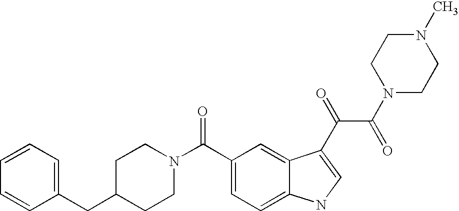 |
| 118 | 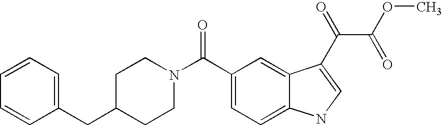 |
| 119 | 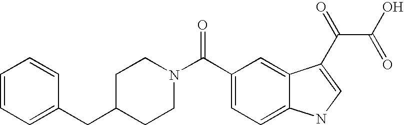 |
| 120 | 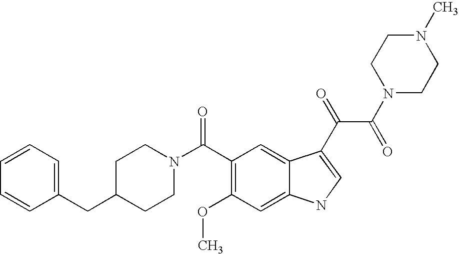 |
| 121 | 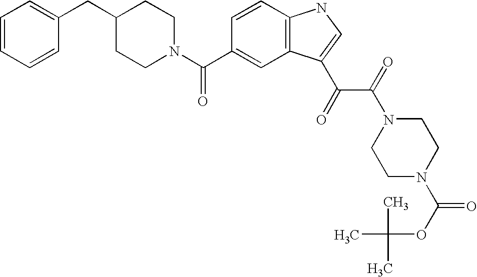 |
| 122 | 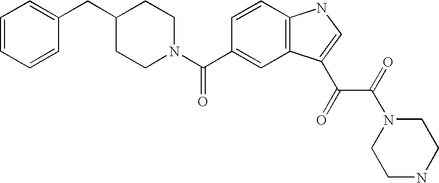 |
| 123 | 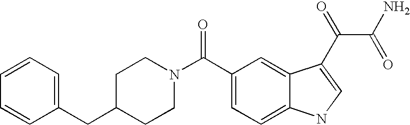 |
| 124 | 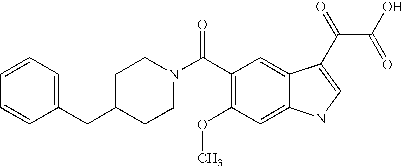 |
| 125 | 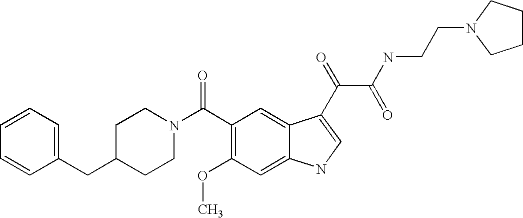 |
| 126 | 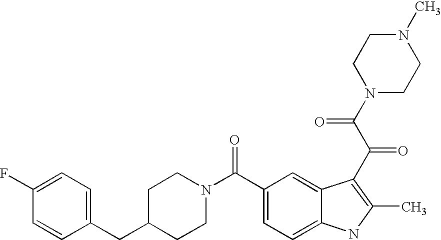 |
| 127 | 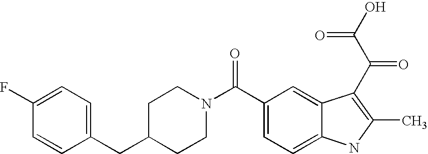 |
| 128 | 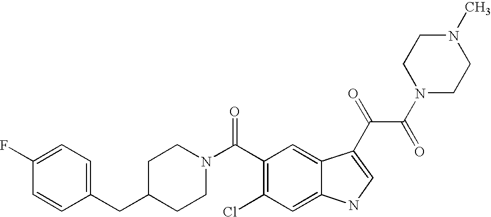 |
| 129 | 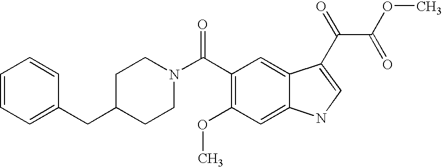 |
| 130 | 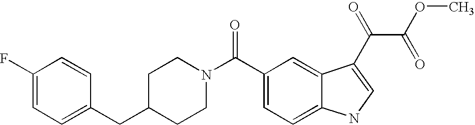 |
| 131 | 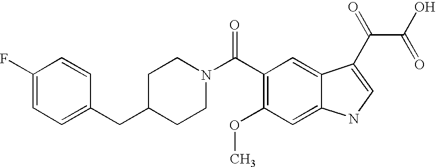 |
| 132 | 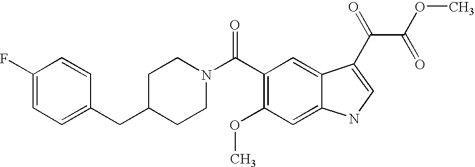 |
| 133 | 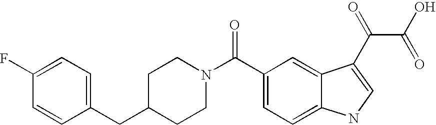 |
| 134 | 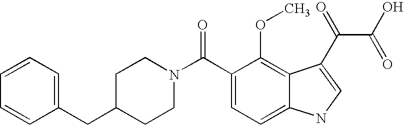 |
| 135 | 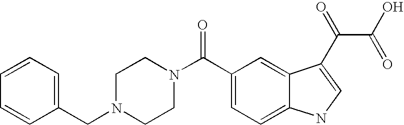 |
| 136 | 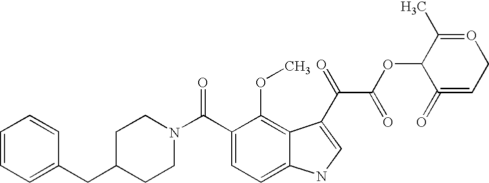 |
| 137 | 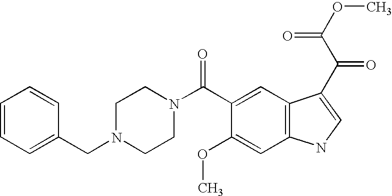 |
| 138 | 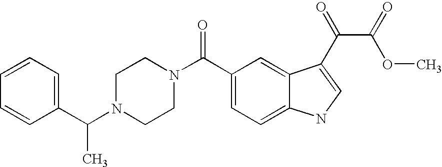 |
| 139 | 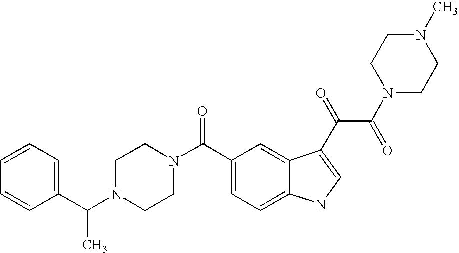 |
| 140 | 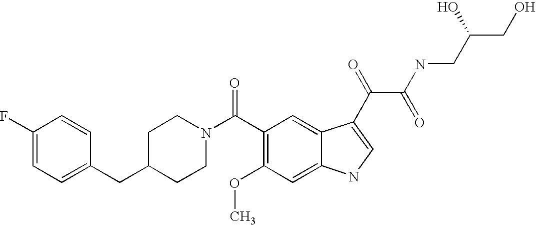 |
| 141 | 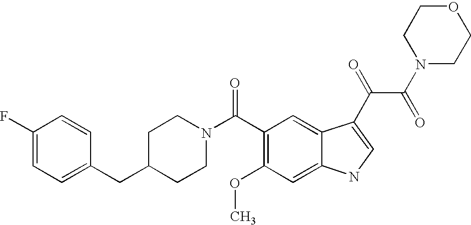 |
| 142 | 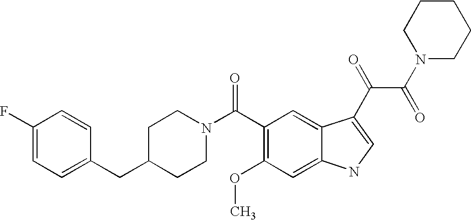 |
| 143 | 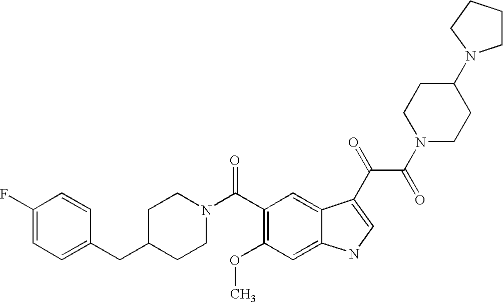 |
| 144 | 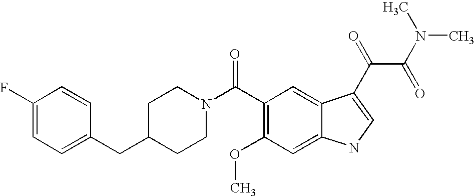 |
| 145 | 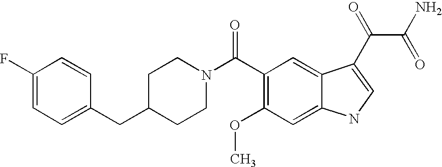 |
| 146 | 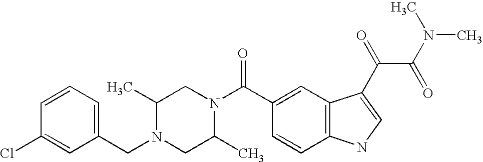 |
| 147 | 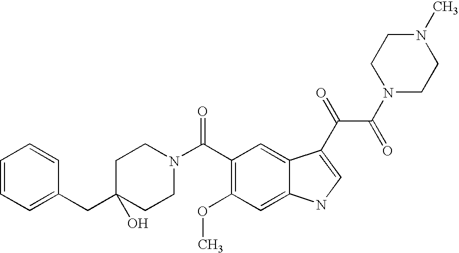 |
| 148 | 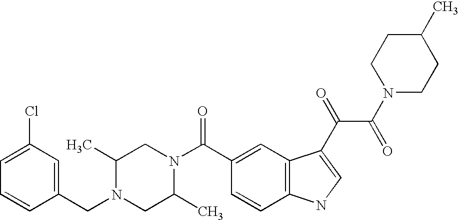 |
| 149 | 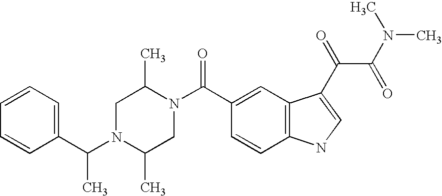 |
| 150 | 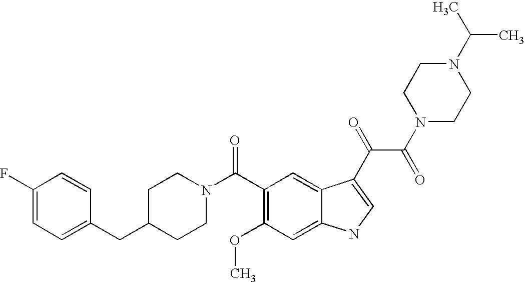 |
| 151 | 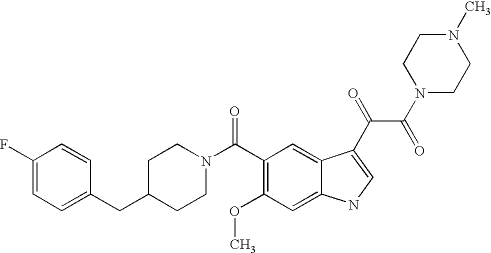 |
| 152 | 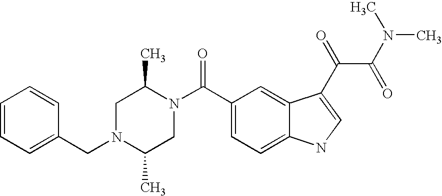 |
| 153 | 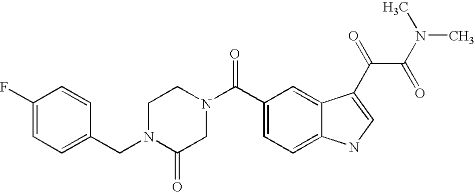 |
| 154 | 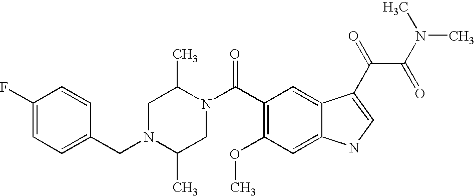 |
| 155 | 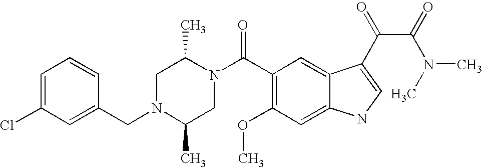 |
| 156 | 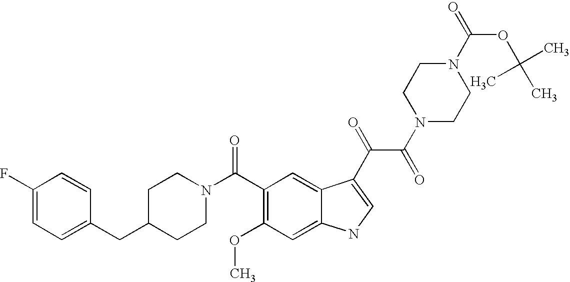 |
| 157 | 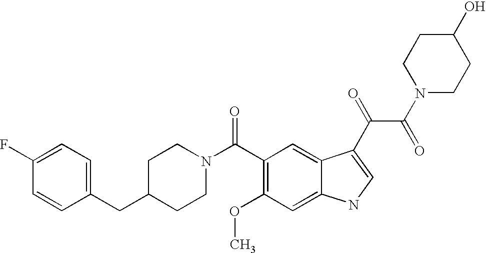 |
| 158 | 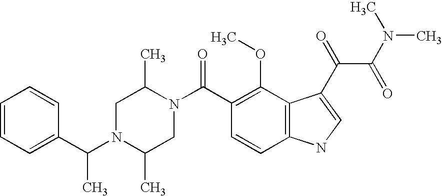 |
| 159 | 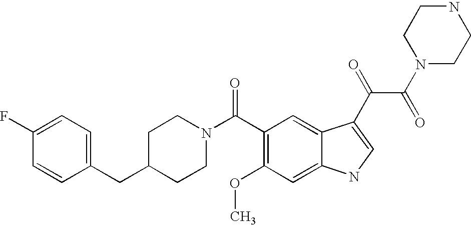 |
| 160 | 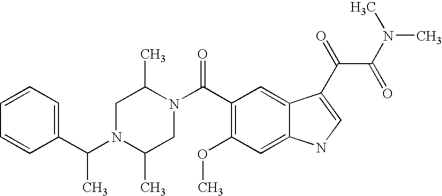 |
| 161 | 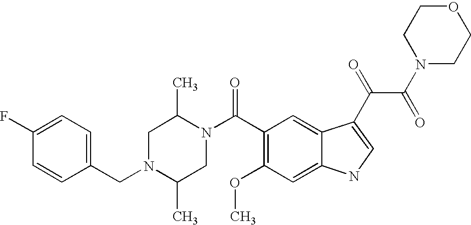 |
| 162 | 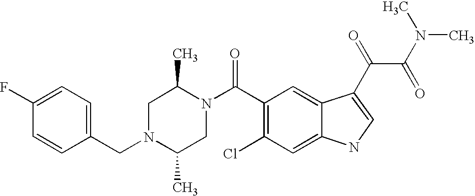 |
| 163 | 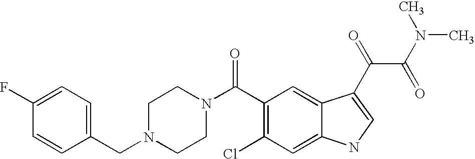 |
| 164 | 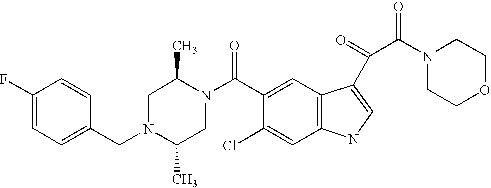 |
| 165 | 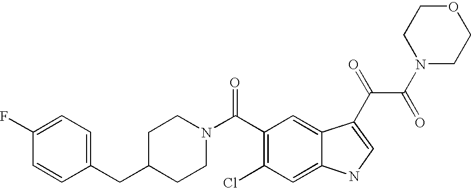 |
| 166 | 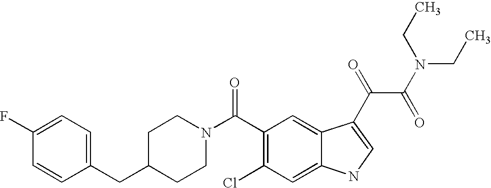 |
| 167 | 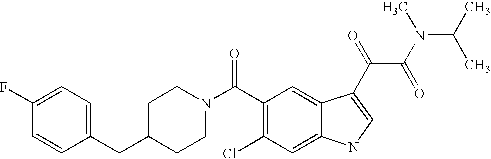 |
| 168 | 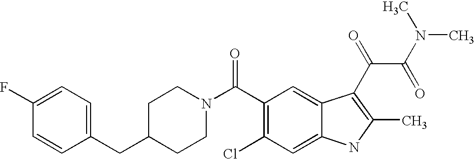 |
| 169 | 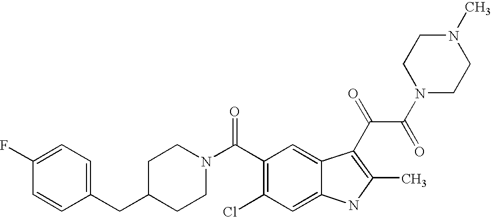 |

Compounds useful in the practice of the present invention also include, but are not limited to, compounds of formulas:

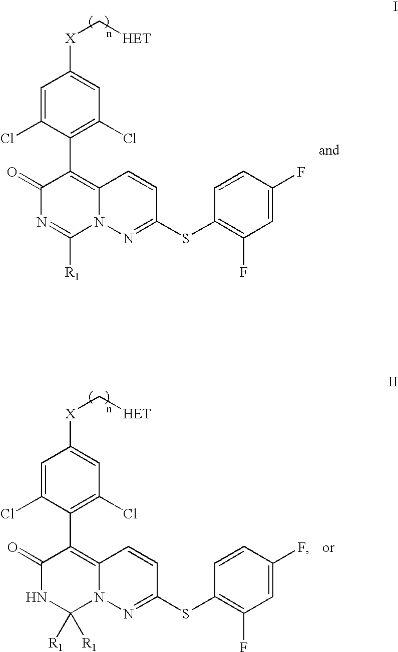

pharmaceutically acceptable salts thereof, wherein

HET is a 5-7 membered heterocycle with 1 to 4 N, S or O atoms, which heterocycle is substituted with 1 to 3 C₁-C₄ branched or straight chain alkyl groups. HET can optionally be substituted with halo, cyano, N(R′)₂, OR′, CO₂R′, CON(R′)₂, and SO₂N(R²)₂;

X is O or NR′;

n is 1 to 3;

R′ is selected from hydrogen, (C₁-C₃)-alkyl, (C₂-C₃)-alkenyl or alkynyl, phenyl or phenyl substituted with 1 to 3 substituents independently selected from halo, methoxy, cyano, nitro, amino, hydroxy, methyl or ethyl; or a 5-6 membered heterocyclic ring system optionally substituted with 1 to 3 substituents independently selected from halo, methoxy, cyano, nitro, amino, hydroxy, methyl or ethyl;

R₁ is selected from hydrogen, (C₁-C₃)-alkyl, hydroxy, or (C₁-C₃)-alkoxy;

R₂ is selected from hydrogen, (C₁-C₃)-alkyl, or (C₁-C₃)-alkenyloxy; each optionally substituted with —N(R′)₂, —OR′, —SR′, —C(O)—N(R′)₂, —S(O₂)—N(R′)₂, —C(O)—OR′, or R³; and

R³ is selected from 5-6 membered aromatic carbocyclic or heterocyclic ring systems.

Compounds useful in the practice of the present invention also include, but are not limited to, compounds of formulas: 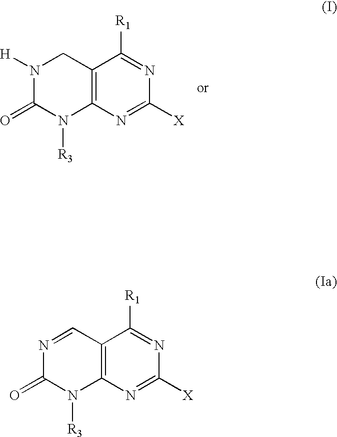
wherein

R₁ is an aryl or heteroaryl ring, which ring is optionally substituted;

R₂ is hydrogen, C₁₋₁₀ alkyl, C₃₋₇ cycloalkyl, C₃₋₇ cycloalkylC₁₋₁₀ alkyl, aryl, arylC₁₋₁₀ alkyl, heteroaryl, heteroarylC₁₋₁₀ alkyl, heterocyclic, or a heterocyclylC₁₋₁₀ alkyl moiety; and wherein each of these moieties, excluding hydrogen, are optionally substituted;

R₃ is a C₁₋₁₀ alkyl, C₃₋₇cycloalkyl, C₃₋₇ cycloalkylC₁₋₁₀alkyl, arylC₁₋₁₀alkyl, heteroaryl C₁₋₁₀alkyl, or heterocyclylC₁₋₁₀ alkyl moiety; and wherein each of these moieties are optionally substituted;

X is R₂, OR₂, S(O)ₘR₂ or (CH₂)ₙNR₄R₁₄, or (CH₂)ₙNR₂R₄;

n is 0 or an integer having a value of 1 to 10;

m is 0 or an integer having a value of 1 or 2;

R₄ and R₁₄ are each independently selected from hydrogen, optionally substituted C₁₋₁₄ alkyl, optionally substituted aryl, or an optionally substituted arylC₁₋₄alkyl, or R₄ and R₁₄ together with the nitrogen to which they are attached form a heterocyclic ring of 5 to 7 members, which ring optionally contains an additional heteroatom selected from oxygen, sulfur or NR₉, and which ring can be optionally substituted;

R₆ is hydrogen, C₁₋₁₀ alkyl, C₃₋₇ cycloalkyl, heterocyclyl, heterocyclylC₁₋₁₀alkyl, aryl, arylC₁₋₁₀ alkyl, heteroaryl or a heteroarylC₁₋₁₀ alkyl moiety; and wherein each of these moieties, excluding hydrogen, can be optionally substituted;

R₉ is hydrogen, C(Z)R₆, optionally substituted C₁₋₁₀ alkyl, optionally substituted aryl or optionally substituted arylC₁₋₄ alkyl;

Z is oxygen or sulfur;

or a pharmaceutically acceptable salt thereof.

Compounds useful in the practice of the present invention also include, but are not limited to, compounds of formulas: 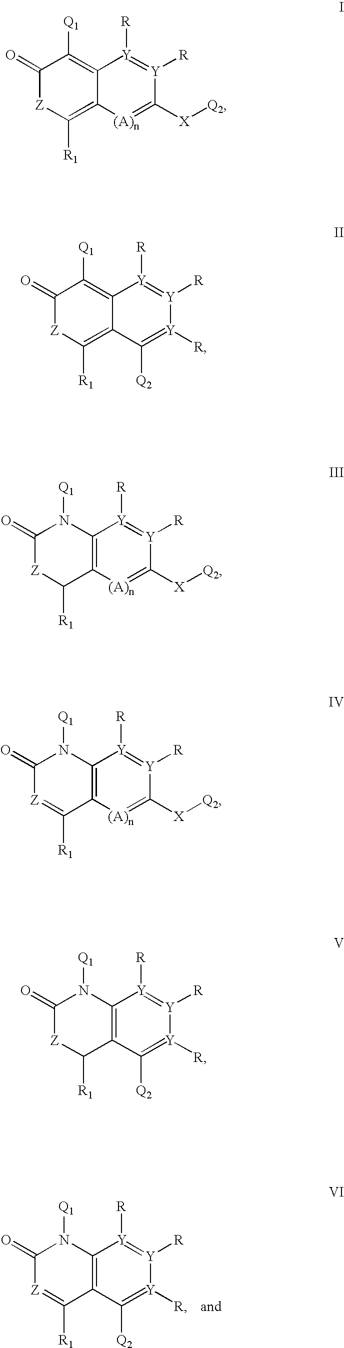 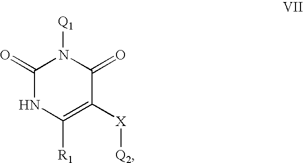
or pharmaceutically acceptable salts thereof, wherein

each of Q₁ and Q₂ are independently selected from 5-6 membered aromatic carbocyclic or heterocyclic ring systems, or 8-10 membered bicyclic ring systems comprising aromatic carbocyclic rings, aromatic heterocyclic rings or a combination of an aromatic carbocyclic ring and an aromatic heterocyclic ring;

the rings that make up Q₁ are substituted with 1 to 4 substituents, each of which is independently selected from halo; C₁-C₃ alkyl optionally substituted with NR′₂, OR′, CO₂R′ or CONR′₂; (C₁-C₃)-alkoxy optionally substituted with NR′₂, OR′, CO₂R′ or CONR′₂; NR′₂; OCF₃; CF₃; NO₂; CO₂R′; CONR′; SR′; S(O₂)N(R′)₂; SCF₃; CN; N(R′)C(O)R⁴; N(R′)C(O)OR⁴; N(R′)C(O)C(O)R⁴; N(R′)S(O₂)R⁴; N(R′)R⁴; N(R⁴)₂; OR⁴; OC(O)R⁴; OP(O)₃H₂; or N═C—N (R′)₂;

the rings that make up Q₂ are optionally substituted with up to 4 substituents, each of which is independently selected from halo; C₁-C₃ straight or branched alkyl optionally substituted with NR′₂, OR′, CO₂R′, S(O₂)N(R′)₂, N═C—N(R′)₂, R³, or CONR′₂; (C₁-C₃)-alkoxy optionally substituted with NR′₂, OR′, CO₂R′, S(O₂)N(R′)₂, N═C—N(R′)₂, R³, or CONR′₂; NR′₂, OCF₃; CF₃; NO₂; CO₂R′; CONR′; R³; OR³; NR³; SR³; C(O)R³; C(O)N(R′)R³; C(O)OR³; SR′; S(O₂)N(R′)₂; SCF₃; N═C—N(R′)₂; or CN;

R′ is selected from hydrogen, (C₁-C₃)-alkyl; (C₂-C₃)-alkenyl; (C₂-C₃) alkynyl; phenyl substituted with 1 to 3 substituents independently selected from halo, methoxy, cyano, nitro, amino, hydroxy, methyl or ethyl;

R³ is selected from 5-6 membered aromatic carbocyclic or heterocyclic ring systems;

R⁴ is (C₁-C₄)-alkyl optionally substituted with N(R′)₂, OR′, CO₂R′, CON(R′)₂, or SO₂N(R²)₂; or a 5-6 membered carbocyclic or heterocyclic ring system optionally substituted with N(R′)₂, OR′, CO₂R′, CON(R′)₂, or SO₂N(R²)₂; X, if present, is selected from —S—, —O—, —S(O₂)—, —S(O)—, —S(O₂)—N(R²)—, —N(R²)—S(O₂)—, —N(R²)—C(O)O—, —O—C(O)—N(R²), —C(O)—, —C(O)O—, —O—C(O)—, —C(O)—N(R²)—, —N(R²)—C(O)—, —N(R²)—, —C(R²₂—, or —C(OR²)₂—;

each R is independently selected from hydrogen, —R², —N(R²)₂, —OR², SR², —C(O)—N(R²)₂, —S(O₂)—N(R²)₂, or —C(O)—OR², wherein two adjacent R are optionally bound to one another and, together with each Y to which they are respectively bound, form a 4-8 membered carbocyclic or heterocyclic ring;

R² is selected from hydrogen, (C₁-C₃)-alkyl, or (C₁-C₃)-alkenyl; each optionally substituted with —N(R′)₂, —OR′, SR′, —C(O)—N(R′)₂, —S(O₂)—N(R′)₂, —C(O)—OR′, or R³;

Y is N or C;

Z, if present, is N, NH, or, if chemically feasible, O;

A, if present, is N or CR′;

n is 0 or 1; and

R₁ is selected from hydrogen, (C₁-C₃)-alkyl, hydroxy, or (C₁-C₃)-alkoxy. Compounds useful in the practice of the present invention also include, but are not limited to, compounds of formula: 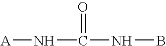
wherein A is 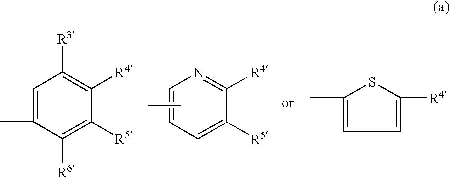
wherein

R^{3′}, R^{4′}, R^{5′} are each independently HOURS, C₁₋₁₀-alkyl, optionally substituted by halogen up to perhalo, C₁₋₁₀ alkoxy, optionally substituted by halogen, up to perhaloalkoxy, halogen; NO₂ or NH₂;

R^{6′} is HOURS, C₁₋₁₀-alkyl, C₁₋₁₀ alkoxy, —NHCOR¹; —NR¹COR¹; NO₂;

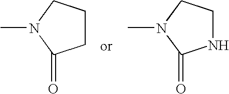

one of R^{4′}, R^{5′}, or R^{6′} can be —X—Y; or

2 adjacent R^{4′}—R^{6′} can together be an aryl or heteroaryl ring with 5-12 atoms, optionally substituted by C₁₋₁₀-alkyl, C₁₋₁₀ alkoxy, C₃₋₁₀ cycloalkyl, C₂₋₁₀ alkenyl, C₁₋₁₀ alkanoyl, C₆₋₁₂ aryl, C₅₋₁₂ heteroaryl or C₆₋₁₂ arakyl;

R¹ is C₁₋₁₀-alkyl optionally substituted by halogen, up to perhalo;

X is —CH₂—, —S—, —N(CH₃)—, —NHC(O)—, —CH₂—S—, —S—CH₂—, —C(O)—, or —O—;

X is additionally a single bond where Y is pyridyl;

Y is phenyl, pyridyl, naphthyl, pyridone, pyrazine, benzodioxane, benzopyridine, pyrimidine or benzothiazole, each optionally substituted by C₁₋₁₀-alkyl, C₁₋₁₀-alkoxy, halogen, OH, —SCH₃ or NO₂ or, where Y is phenyl, by 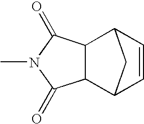
or a pharmaceutically-acceptable salt thereof; 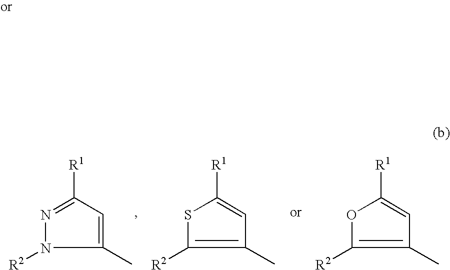
wherein

R¹ is selected from the group consisting of C₃-C₁₀ alkyl, C₃-C₁₀ cycloalkyl, up to per-halo substituted C₁-C₁₀ alkyl and up to per-halosubstituted C₃-C₁₀ cycloalkyl; and

R² is C₆-C₁₄ aryl, C₃-C₁₄ heteroaryl, substituted C₆-C₁₄ aryl or substituted C₃-C₁₄ heteroaryl;

wherein if R² is a substituted group, it is preferably substituted by one or more substituents independently selected from the group consisting of halogen, up to per-halosubstitution, and Vₙ, where n=0-3 and each V is independently selected from the group consisting of —CN, —OC(O)NR⁵R⁵′, —CO₂R⁵, —C(O)NR⁵R⁵′, —OR⁵, —SR⁵, —NR⁵R^{5′}, —C(O)R⁵, —NR⁵C(O)OR^{5′}, —SO₂R⁵—SOR⁵, —NR⁵C(O)R^{5′}, —NO₂, C₁-C₁₀ alkyl, C₃-C₁₀ cycloalkyl, C₆-C₁₄ aryl, C₃-C₁₃ heteroaryl, C₇-C₂₄ alkaryl, C₄-C₂₄ alkheteroaryl, substituted C₁-C₁₀ alkyl, substituted C₃-C₁₀ cycloalkyl, substituted C₆-C₁₄ aryl, substituted C₃-C₁₃ heteroaryl, substituted C₇-C₂₄ alkaryl and substituted C₄-C₂₄ alkheteroaryl;

wherein if V is a substituted group, it is substituted by one or more substituents independently selected from the group consisting of halogen, up to per-halosubstitution, —CN, —CO₂R⁵, —C(O)R⁵, —C(O)NR⁵R^{5′}, —NR⁵R^{5′}, —OR⁵, —SR⁵, —NR⁵C(O)R^{5′}, —NR⁵C(O)OR^{5′} and —NO₂; and

R⁵ and R^{5′} are independently selected form the group consisting of HOURS, C₁-C₁₀ alkyl, C₃-C₁₀ cycloalkyl, C₆-C₁₄ aryl, C₃-C₁₃ heteroaryl, C₇-C₂₄ alkaryl, C₄-C₂₃ alkheteroaryl, up to per-halosubstituted C₁-C₁₀ alkyl, up to per-halosubstituted C₃-C₁₀ cycloalkyl, up to per-halosubstituted C₆-C₁₄ aryl and up to per-halosubstituted C₃-C₁₃ heteroaryl;

or a pharmaceutically-acceptable salt thereof;

or

(c) a substituted moiety of up to 40 carbon atoms of the formula: —L-(M-L¹)_{q}, where L is a 5- or 6-membered cyclic structure bound directly to D, L¹, comprises a substituted cyclic moiety having at least 5 members, M is a bridging group having at least one atom, q is an integer of from 1-3; and each cyclic structure of L and L¹ contains 0-4 members of the group consisting of nitrogen, oxygen and sulfur;

L¹ is substituted by at least one substituent selected from the group consisting of —SO₂Rₓ, —C(O)Rₓ and —C(NR_{y})R_{z};

R_{y} is hydrogen or a carbon-based moiety of up to 24 carbon atoms optionally containing heteroatoms selected from N, S and O and optionally halosubstituted, up to perhalo;

R_{z} is hydrogen or a carbon-based moiety of up to 30 carbon atoms optionally containing heteroatoms selected from N, S and O and optionally substituted by halogen, hydroxy and carbon-based substituents of up to 24 carbon atoms, which optionally contain heteroatoms selected from N, S and O and are optionally substituted by halogen; and

Rₓ is R_{z} or NRₐR_{b} where Rₐ and R_{b} are

i) independently hydrogen,
a carbon-based moiety of up to 30 carbon atoms optionally containing heteroatoms selected from N, S and O and optionally substituted by halogen, hydroxy and carbon-based substituents of up to 24 carbon atoms, which optionally contain heteroatoms selected from N, S and O and are optionally substituted by halogen, or
—OSi(R_{f})₃ where R_{f} is hydrogen or a carbon-based moiety of up to 24 carbon atoms optionally containing heteroatoms selected from N, S and O and optionally substituted by halogen, hydroxy and carbon-based substituents of up to 24 carbon atoms, which optionally contain heteroatoms selected from N, S and O and are optionally substituted by halogen; or

ii) Rₐ and R_{b} together form a 5-7 member heterocyclic structure of 1-3 heteroatoms selected from N, S and O, or a substituted 5-7 member heterocyclic structure of 1-3 heteroatoms selected from N, S and O, substituted by halogen, hydroxy or carbon-based substituents of up to 24 carbon atoms, which optionally contain heteroatoms selected from N, S and O and are optionally substituted by halogen; or

iii) one of Rₐ or R_{b} is —C(O)—, a C₁-C₅ divalent alkylene group or a substituted C₁-C₅ divalent alkylene group bound to the moiety L to form a cyclic structure with at least 5 members, wherein the substituents of the substituted C₁-C₅ divalent alkylene group are selected from the group consisting of halogen, hydroxy, and carbon-based substituents of up to 24 carbon atoms, which optionally contain heteroatoms selected from N, S and O and are optionally substituted by halogen;

or a pharmaceutically-acceptable salt thereof; and

B is an unsubstituted or substituted, up to tricyclic, aryl or heteroaryl moiety with up to 30 carbon atoms with at least one 5- or 6-membered aromatic structure containing 0-4 members of the group consisting of nitrogen, oxygen and sulfur;

wherein if B is substituted, it is substituted by one or more substituents selected from the group consisting of halogen, up to per-halo, and Wₙ, wherein n is 0-3 and each W is independently selected from the group consisting of —CN, —CO₂R⁷, —C(O)NR⁷R⁷, —C(O)R⁷, —NO₂, —OR⁷, —SR⁷, —NR⁷R⁷, —NR⁷C(O)OR⁷, —NR⁷C(O)R⁷, C₁-C₁₀ alkyl, C₂₋₁₀-alkenyl, C₁-₁₀-alkoxy, C₃-C₁₀ cycloalkyl, C₆-C₁₄ aryl, C₇-C₂₄ alkaryl, C₃-C₁₃ heteroaryl, C₄-C₂₃ alkheteroaryl, substituted C₁-C₁₀ alkyl, substituted C₂₋₁₀-alkenyl, substituted C₁-₁₀-alkoxy, substituted C₃-C₁₀ cycloalkyl, substituted C₄-C₂₃ alkheteroaryl and —Q—Ar;

wherein if W is a substituted group, it is substituted by one or more substituents independently selected from the group consisting of —CN, —CO₂R⁷, —C(O)NR⁷R⁷, —C(O)R⁷, —NO₂, —OR⁷, —SR⁷, —NR⁷R⁷, —NR⁷C(O)OR⁷, —NR⁷C(O)R⁷ and halogen up to per-halo;

wherein each R⁷ is independently selected from HOURS, C₁-C₁₀ alkyl, C₂-₁₀-alkenyl, C₃-C₁₀ cycloalkyl, C₆-C₁₄ aryl, C₃-C₁₃ heteroaryl, C₇-C₂₄ alkaryl, C₄-C₂₃ alkheteroaryl, up to per-halosubstituted C₁-C₁₀ alkyl, up to per-halosubstituted C₂-₁₀-alkenyl, up to per-halosubstituted C₃-C₁₀ cycloalkyl, up to per- halosubstituted C₆-C₁₄ aryl and up to per-halosubstituted C₃-C₁₃ heteroaryl;

wherein Q is —O—, —S—, —N(R)⁷, —(CH₂)—ₘ, —C(O)—, —CH(OH)—, —NR⁷C(O)NR⁷R⁷—, —NR⁷C(O)—, —C(O)NR⁷—, —(CH₂)ₘO—, —(CH₂)ₘS—, —(CH₂)ₘN(R⁷)—, —O(CH₂)ₘ—, —CHX^{a}, —CX^{a}₂—, —S—(CH₂)ₘ— and —N(R⁷)(CH₂)ₘ—, where m =1-3, and X^{a} is halogen; and

Ar is a 5-10 member aromatic structure containing 0-4 members of the group consisting of nitrogen, oxygen and sulfur, which is unsubstituted or substituted by halogen up to per-halosubstitution and optionally substituted by Zₙ₁, wherein n1 is 0 to 3 and each Z substituent is independently selected from the group consisting of —CN, —CO₂R⁷, —C(O)NR⁷R⁷, —C(O)—NR⁷, —NO₂, —OR⁷, —SR⁷, —NR⁷R⁷, —NR⁷C(O)OR⁷, C(O)R⁷, —NR⁷C(O)R⁷, C₁-C₁₀ alkyl, C₃-C₁₀ cycloalkyl, C₆-C₁₄ aryl, C₃-C₁₃ heteroaryl, C₇-C₂₄ alkaryl, C₄-C₂₃ alkheteroaryl, substituted C₁-C₁₀ alkyl, substituted C₃-C₁₀ cycloalkyl, substituted C₇-C₂₄ alkaryl and substituted C₄-C₂₃ alkheteroaryl; wherein the one or more substituents of Z are independently selected from the group consisting of —CN, —CO₂R⁷, —C(O)NR⁷R⁷, —OR⁷, —SR⁷, —NO₂, —NR⁷R⁷, —NR⁷C(O)R⁷ and —NR⁷C(O)OR⁷;
or a pharmaceutically-acceptable salt thereof

Exemplary compounds of these formulas include:
N-(5-tert-butyl-2-methoxyphenyl)-N′-(4-phenyloxyphenyl)urea;
N-(5-tert-butyl-2-methoxyphenyl)-N′-(4-(4-methoxyphenyloxy)phenyl)urea; N-(5-tert-butyl-2-methoxyphenyl)-N′-(4-(4-pyridinyloxy)phenyl)urea;
N-(5-tert-butyl-2-methoxyphenyl)-N′-(4-(4-pyridinylmethyl)phenyl)urea;
N-(5-tert-butyl-2-methoxyphenyl)-N′-(4-(4-pyridinylthio)phenyl)urea;
N-(5-tert-butyl-2-methoxyphenyl)-N′-(4-(4-(4,7-methano-1H-isoindole-1, 3(2H)-dionyl)methyl)phenyl)urea;
N-(5-tert-butyl-2-phenylphenyl)-N′-(2,3-dichlorophenyl)urea;
N-(5-tert-butyl-2-(3-thienyl)phenyl)-N′-(2,3-dichlorophenyl)urea;
N-(5-tert-butyl-2-(N-methylaminocarbonyl)methoxyphenyl)-N′-(2,3-dichlorophenyl)urea;
N-(5-tert-butyl-2-(N-methylaminocarbonyl)methoxyphenyl)-N′-(1-naphthyl)urea;
N-(5-tert-butyl-2-(N-morpholinocarbonyl)methoxyphenyl)-N′-(2,3-dichlorophenyl)urea;
N-(5-tert-butyl-2-(N-morpholinocarbonyl)methoxyphenyl)-N′-(1-naphthyl)urea;
N-(5-tert-butyl-2-(3-tetrahydrofuranyloxy)phenyl)-N′-(2,3-dichlorophenyl)urea;
N-(5-tert-butyl-2-methoxyphenyl)-N′-(4-(3-pyridinyl)methylphenyl)urea;
N-(5-trifluoromethyl-2-methoxyphenyl)-N′-(4-methylphenyl)urea;
N-(5-trifluoromethyl-2-methoxyphenyl)-N′-(4-methyl-2-fluorophenyl)urea;
N-(5-trifluoromethyl-2-methoxyphenyl)-N′-(4-fluoro-3-chlorophenyl)urea;
N-(5-trifluoromethyl-2-methoxyphenyl)-N′-(4-methyl-3-chlorophenyl)urea;
N-(5-trifluoromethyl-2-methoxyphenyl)-N′-(4-methyl-3-fluorophenyl)urea;
N-(5-trifluoromethyl-2-methoxyphenyl)-N′-(2,4-difluorophenyl)urea;
N-(5-trifluoromethyl-2-methoxyphenyl)-N′-(4-phenyloxy-3,5-dichlorophenyl)urea;
N-(5-trifluoromethyl-2-methoxyphenyl)-N′-(4-(4-pyridinylmethyl)phenyl)urea;
N-(5-trifluoromethyl-2-methoxyphenyl)-N′-(4-(4-pyridinylthio)phenyl)urea;
N-(5-trifluoromethyl-2-methoxyphenyl)-N′-(4-(4-pyridinyloxy)phenyl)urea;
N-(5-trifluoromethyl-2-methoxyphenyl)-N′-(3-(4-pyridinylthio)phenyl)urea;
N-(5-trifluoromethyl-2-methoxyphenyl)-N′-(4-(3-(N-methylaminocarbonyl)phenyloxy)phenyl)urea;
N-(5-fluorosulfonyl)-2-methoxyphenyl)-N′-(4-methylphenyl)urea;
N-(5-(difluromethanesulfonyl)-2-methoxyphenyl)-N′-(4-methylphenyl)urea;
N-(5-(difluoromethanesulfonyl)-2-methoxyphenyl)-N′-(4-fluorophenyl)urea;
N-(5-(difluoromethanesulfonyl)-2-methoxyphenyl)-N′-(4-methyl-2-fluorophenyl)urea;
N-(5-(difluoromethanesulfonyl)-2-methoxyphenyl)-N′-(4-methyl-3-fluorophenyl)urea;
N-(5-(difluoromethanesulfonyl)-2-methoxyphenyl)-N′-(4-methyl-3-chlorophenyl)urea;
N-(5-(difluoromethanesulfonyl)-2-methoxyphenyl)-N′-(4-fluoro-3-chlorophenyl)urea;
N-(5-(difluoromethanesulfonyl)-2-methoxyphenyl)-N′-(4-fluoro-3-methylphenyl)urea;
N-(5-(difluoromethanesulfonyl)-2-methoxyphenyl)-N′-(2,3-dimethylphenyl)urea;
N-(5-(trifluoromethanesulfonyl)-2-methoxphenyl)-N′-(4-methylphenyl)urea;
N-(3-methoxy-2-naphthyl)-N′-(2-fluorophenyl)urea;
N-(3-methoxy-2-naphthyl)-N′-(4-methylphenyl)urea;
N-(3-methoxy-2-naphthyl)-N′-(3-fluorophenyl)urea;
N-(3-methoxy-2-naphthyl)-N′-(4-methyl-3-fluorophenyl)urea;
N-(3-methoxy-2-naphthyl)-N′-(2,3-dimethylphenyl)urea;
N-(3-methoxy-2-naphthyl)-N′-(1-naphthyl)urea;
N-(3-methoxy-2-naphthyl)-N′-(4-(4-pyridinylmethyl)phenyl)urea;
N-(3-methoxy-2-naphthyl)-N′-(4-(4-pyridinylthio)phenyl)urea;
N-(3-methoxy-2-naphthyl)-N′-(4-(4-methoxyphenyloxy)phenyl)urea;
N-(3-methoxy-2-naphthyl)-N′-(4-(4-(4,7-methano-1H-isoindole-1,3(2H)-dionyl)methyl)phenyl)urea;
N-(2-hydroxy-4-nitro-5-chlorophenyl)-N′-(phenyl)urea;
N-(2-hydroxy-4-nitro-5-chlorophenyl)-N′-(4-(4-pyridinylmethyl)phenyl)urea;
   and pharmaceutically acceptable salts thereof.

Such compounds are described in published PCT applications WO 96/21452, WO 96/40143, WO 97/25046, WO 97/35856, WO 98/25619, WO 98/56377, WO 98/57966, WO 99/32110, WO 99/32121, WO 99/32463, WO 99/61440, WO 99/64400, WO 00/10563, WO 00/17204, WO 00/19824, WO 00/41698, WO 00/64422, WO 00/71535, WO 01/38324, WO 01/64679, WO 01/66539, and WO 01/66540, each of which is herein incorporated by reference.

In all instances herein where there is an alkenyl or alkynyl moiety as a substituent group, the unsaturated linkage, i.e., the vinylene or acetylene linkage, is preferably not directly attached to the nitrogen, oxygen or sulfur moieties, for instance in ORf, or for certain R₂ moieties.

As used herein, “optionally substituted” unless specifically defined shall mean such groups as halogen, such as fluorine, chlorine, bromine or iodine; hydroxy; hydroxy-substituted C₁₋₁₀-alkyl; C₁₋₁₀ alkoxy, such as methoxy or ethoxy; S(O)ₘ alkyl, wherein m is 0, 1 or 2, such as methyl thio, methylsulfinyl or methyl sulfonyl; amino, mono and di-substituted amino, such as in the NR₇R₁₇ group; or where the R₇R₁₇ can together with the nitrogen to which they are attached cyclize to form a 5- to 7-membered ring which optionally includes an additional heteroatom selected from O, N, and S; C₁₋₁₀ alkyl, cycloalkyl, or cycloalkyl alkyl group, such as methyl, ethyl, propyl, isopropyl, t-butyl, etc. or cyclopropyl methyl; halo-substituted C₁₋₁₀ alkyl, such as CF₃; an optionally substituted aryl, such as phenyl, or an optionally substituted arylalkyl, such as benzyl or phenethyl, wherein these aryl moieties can also be substituted one to two times by halogen; hydroxy; hydroxy-substituted alkyl; C₁₋₁₀ alkoxy; S(O)ₘ alkyl; amino, mono- and di-substituted amino, such as in the NR₇R₁₇ group; alkyl, or CF₃.

Inhibitors useful in the present invention can be used with any pharmaceutically acceptable salt. The term “pharmaceutically acceptable salts” refers to salts prepared from pharmaceutically acceptable non-toxic bases or acids. When the compound utilized by the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Basic salts of inorganic and organic acids also include as hydrochloric acid, hydrobromic acid, sulphuric acid, phosphoric acid, methane sulphonic acid, ethane sulphonic acid, acetic acid, malic acid, tartaric acid, citric acid, lactic acid, oxalic acid, succinic acid, fumaric acid, maleic acid, benzoic acid, salicylic acid, phenylacetic acid and mandelic acid. In addition, pharmaceutically-acceptable salts of the above-described compounds can also be formed with a pharmaceutically-acceptable cation, for instance, if a substituent group comprises a carboxy moiety. Suitable pharmaceutically-acceptable cations are well known to those skilled in the art and include alkaline, alkaline earth, ammonium and quaternary ammonium cations.

Other pharmaceutically acceptable organic non-toxic bases from which salts can be formed include ion exchange resins such as, for example, arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

The inhibitors of p38 MAP kinase can be used as single therapeutic agents or in combination with other therapeutic agents. Drugs that could be usefully combined with these compounds include monoclonal antibodies targeting cells of the immune system, antibodies or soluble receptors or receptor fusion proteins targeting immune or non-immune cytokines, and small molecule inhibitors of cell division, protein synthesis, or mRNA transcription or translation, or inhibitors of immune cell differentiation, activation, or function (e.g., cytokine secretion).

Other compounds useful in the practice of the present invention include, but are not limited to, the compounds shown in Table B, below.

TABLE B

| | Citations, each of which is herein |
|---|---|
| Chemical Structure | incorporated by reference |
| 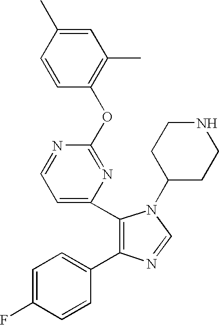 | WO-00166539, WO-00166540, WO-00166539, WO-00166540, WO-00064422, WO-00019824, WO-00010563, WO-09961440, WO-09932121, WO-09857966, WO-09856377, WO-09825619, WO-05756499, WO-09735856, WO-09725046, WO-09640143, WO-09621452; Gallagher, T.F., et. Al., Bioorg. Med. Chem. 5:49 (1997); Adams, J.L., et al., Bioorg. Med. Chem. Lett. 8:3111-3116 (1998) |
| 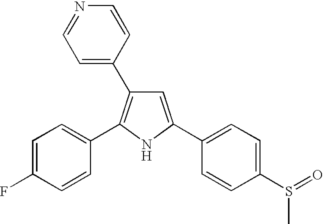 | De Laszlo, S.E., et. Al., Bioorg Med Chem Lett. 8:2698 (1998) |
| 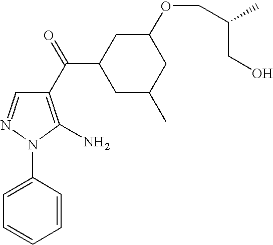 | WO-09957101; Poster presentation at the 5^{th} World Congress on Inflammation, Edinburgh, UK. (2001) |
| 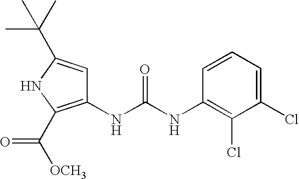 | WO-00041698, WO-09932110, WO-09932463 |
| 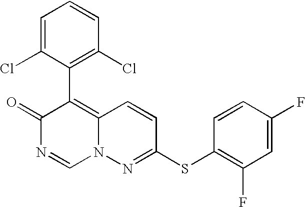 | WO-00017204, WO-09964400 |
| 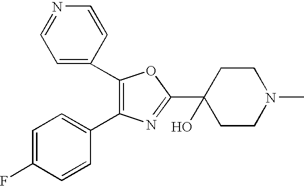 | Revesz. L., et. al., Bioorg Med Chem Lett. 10:1261 (2000) |
| 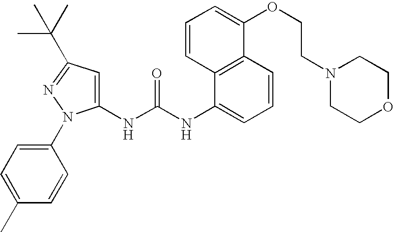 | WO-00207772 |
| 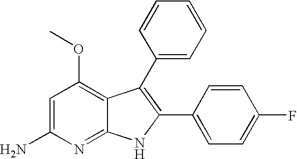 | Fijen, J.W., et al., Clin. Exp. Immunol. 124:16-20 (2001); Wadsworth, S.A., et. al., J. Pharmacol. Expt. Therapeut. 291:680 (1999) |
| 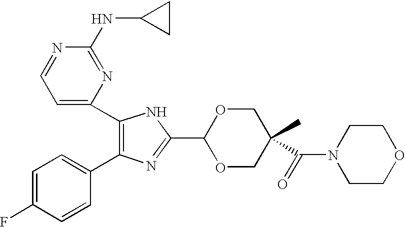 | Collis, A.J., et al.. Bioorg. Med. Chem. Lett. 11:693-696 (2001); McLay, L.M., et al., Bioorg Med Chem 9:537-554 (2001) |
| 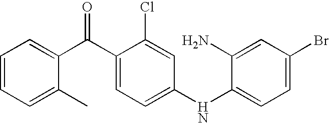 | WO-00110865, WO-00105749 |

All compounds provided herein are intended for guidance and exemplary purposes only. It should be understood that any inhibitor of p38 MAP kinase is useful for the invention provided that it exhibits activity relative to p38 to the extent adequate to exhibit a therapeutic effect in a reasonable dosage and concentration.

Formulations and Methods of Administration

A pharmaceutical composition useful in the present invention comprises a p38 MAP kinase inhibitor (such as those described above) and a pharmaceutically acceptable carrier, excipient, diluent and/or salt.

Pharmaceutically acceptable carrier, diluent, excipient, and/or salt means that the carrier, diluent, excipient and/or salt must be compatible with the other ingredients of the formulation, does not adversely affect the therapeutic benefit of the p38 MAP kinase inhibitor, and is not deleterious to the recipient thereof.

Administration of the compounds or pharmaceutical compositions thereof for practicing the present invention can be by any method that delivers the compounds systemically and/or locally (e.g., at the site of the disease, intravascular, etc.). These methods include oral routes, parenteral routes, intraduodenal routes, and the like. Preferably, the compositions are delivered parenterally.

For topical applications, the compound or pharmaceutical composition thereof can be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax, sugars such as lactose and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Depending on the particular condition, disorder or disease to be treated, additional therapeutic agents can be administered together with the p38 MAP kinase inhibitors. Those additional agents can be administered sequentially in any order, as part of a multiple dosage regimen, from the p38 MAP kinase inhibitor-containing composition (consecutive or intermittent administration). Alternatively, those agents can be part of a single dosage form, mixed together with the p38 MAP kinase inhibitor in a single composition (simultaneous or concurrent administration).

For oral administration, a pharmaceutical composition useful in the invention can take the form of solutions, suspensions, tablets, pills, capsules, powders, granules, semisolids, sustained release formulations, elixirs, aerosols, and the like. Tablets containing various excipients such as sodium citrate, calcium carbonate and calcium phosphate are employed along with various disintegrants such as starch, preferably potato or tapioca starch, and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

The choice of formulation depends on various factors such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The term “parenteral” as used herein refers to modes of administration which include intravenous, intramuscular, intraperitoneal, intrasternal, subcutaneous, intramedullary and intraarticular injection and infusion. Parenteral delivery is a preferred route of delivery for purposes of the present invention. A pharmaceutical composition for parenteral injection can comprise pharmaceutically acceptable sterile aqueous or nonaqueous solutions, dispersions, suspensions or emulsions as well as sterile powders for reconstitution into sterile injectable solutions or dispersions just prior to use. Aqueous solutions are especially suitable for intravenous, intramuscular, subcutaneous and intraperitoneal injection purposes. In this connection, the sterile aqueous media employed are all readily obtainable by standard techniques well-known to those skilled in the art. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

The pharmaceutical compositions useful in the present invention can also contain adjuvants such as, but not limited to, preservatives, wetting agents, emulsifying agents, and dispersing agents. Prevention of the action of microorganisms can be ensured by the inclusion of various antibacterial and antifungal agents, such as for example, paraben, chlorobutanol, phenol sorbic acid, and the like. It can also be desirable to include isotonic agents such as sugars, sodium chloride, and the like. Prolonged absorption of the injectable pharmaceutical form can be brought about by the inclusion of agents that delay absorption such as aluminum monostearate and gelatin.

In some cases, in order to prolong the effect of the drugs, it is desirable to slow the absorption from subcutaneous or intramuscular injection. This can be accomplished by the use of a liquid suspension of crystalline or amorphous material with poor water solubility. The rate of absorption of the drug then depends upon its rate of dissolution which, in turn, can depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide, polyglycolide, and polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use..

Suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

For purposes of transdermal (e.g., topical) administration, dilute sterile, aqueous or partially aqueous solutions (usually in about 0.1% to 5% concentration), otherwise similar to the above parenteral solutions, are prepared.

The pharmaceutical compositions useful in the invention can also be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, and/or other conventional solubilizing or dispersing agents.

In nonpressurized powder compositions, the active ingredients in finely divided form can be used in admixture with a larger-sized pharmaceutically acceptable inert carrier comprising particles having a size, for example, of up to 100 μm in diameter. Suitable inert carriers include sugars such as lactose. Desirably, at least 95% by weight of the particles of the active ingredient have an effective particle size in the range of 0.01 to 10 μm.

Alternatively, the composition can be pressurized and contain a compressed gas, such as, e.g., nitrogen, carbon dioxide or a liquefied gas propellant. The liquefied propellant medium and indeed the total composition are preferably such that the active ingredients do not dissolve therein to any substantial extent. The pressurized composition can also contain a surface active agent. The surface active agent can be a liquid or solid non-ionic surface active agent or can be a solid anionic surface active agent. It is preferred to use the solid anionic surface active agent in the form of a sodium salt.

Compositions for rectal or vaginal administration are preferably suppositories which can be prepared by mixing the compounds of the invention with suitable non-irritating excipients or carriers such as cocoa butter, polyethylene glycol or a suppository wax which are solid at room temperature but liquid at body temperature and therefore melt in the rectum or vaginal cavity and release the drugs.

The compositions useful in the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to the compounds of the invention, stabilizers, preservatives, excipients, and the like. The preferred lipids are the phospholipids and the phosphatidyl cholines (lecithins), both natural and synthetic. Methods to form liposomes are known in the art (see e.g., Prescott, E., Meth. Cell Biol. 14:33 (1976)).

Other pharmaceutically acceptable carrier includes, but is not limited to, a non-toxic solid, semisolid or liquid filler, diluent, encapsulating material or formulation auxiliary of any type, including but not limited to ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Solid pharmaceutical excipients include, but are not limited to, starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients can be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Methods of preparing various pharmaceutical compositions with a certain amount of active ingredient are known, or will be apparent in light of this disclosure, to those skilled in this art. Other suitable pharmaceutical excipients and their formulations are described in Remington's Pharmaceutical Sciences, edited by E. W. Martin, Mack Publishing Company, 19th ed. (1995).

Pharmaceutical compositions useful in the present invention can contain 0.1%-95% of the compound(s) of this invention, preferably 1%-70%. In any event, the composition or formulation to be administered will contain a quantity of a compound(s) according to this invention in an amount effective to treat the condition, disorder or disease of the subject being treated.

One of ordinary skill in the art will appreciate that pharmaceutically effective amounts of the p38 MAP kinase inhibitor can be determined empirically and can be employed in pure form or, where such forms exist, in pharmaceutically acceptable salt, ester or prodrug form. The agents can be administered to a patient as pharmaceutical compositions in combination with one or more pharmaceutically acceptable excipients. It will be understood that, when administered to, for example, a human patient, the total daily usage of the agents or composition of the present invention will be decided within the scope of sound medical judgment by the attending physician. The specific pharmaceutically effective dose level for any particular patient will depend upon a variety of factors: the type and degree of the cellular response to be achieved; activity of the specific agent or composition employed; the specific agents or composition employed; the age, body weight, general health, sex and diet of the patient; the time of administration, route of administration, and rate of excretion of the agent; the duration of the treatment; drugs used in combination or coincidental with the specific agent; and like factors well known in the medical arts. For example, it is well within the skill of the art to start doses of the agents at levels lower than those required to achieve the desired therapeutic effect and to gradually increase the dosages until the desired effect is achieved.

For example, satisfactory results are obtained by oral administration of the compounds at dosages on the order of from 0.05 to 500 mg/kg/day, preferably 0.1 to 100 mg/kg/day, more preferably 1 to 50 mg/kg/day, administered once or, in divided doses, 2 to 4 times per day. On administration parenterally, for example, by i.v. bolus, drip or infusion, dosages on the order of from 0.01 to 1000 mg/kg/day, preferably 0.05 to 500 mg/kg/day, and more preferably 0.1 to 100 mg/kg/day, can be used. Suitable daily dosages for patients are thus on the order of from 2.5 to 500 mg p.o., preferably 5 to 250 mg p.o., more preferably 5 to 100 mg p.o., or on the order of from 0.5 to 250 mg i.v., preferably 2.5 to 125 mg i.v. and more preferably 2.5 to 50 mgi.v.

Dosaging can also be arranged in a patient specific manner to provide a predetermined concentration of the agents in the blood, as determined by techniques accepted and routine in the art (HPLC is preferred). Thus patient dosaging can be adjusted to achieve regular on-going blood levels, as measured by HPLC, on the order of from 50 to 5000 ng/ml, preferably 100 to 2500 ng/ml.

Kits

The invention also relates to combining separate pharmaceutical compositions in kit form. The kit can have a carrier means being compartmentalized in close confinement to receive two or more container means therein, having (1) a first container means containing a pharmaceutically effective amount of a p38 MAP kinase inhibitor and (2) a second container means containing a pharmaceutically effective amount of carrier, excipient or diluent. Optionally, the kit can have additional container mean(s) containing a pharmaceutically effective amount of additional agents.

The kit comprises a container for containing the separate compositions such as a divided bottle or a divided foil packet, however, the separate compositions can also be contained within a single, undivided container. Typically the kit comprises directions for administration of the separate components. The kit form is particularly advantageous when the separate components are preferably administered in different dosage forms (e.g., oral and parenteral) or at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It can be desirable to provide a memory aid on the kit, e.g., in the form of numbers next to the tablets or capsules whereby the numbers correspond with the days of the regimen which the dosage form so specified should be ingested. Another example of such a memory aid is a calendar printed on the card e.g., “First Week, Monday, Tuesday . . . Second Week, Monday, Tuesday. . .” etc. Other variations of memory aids will be readily apparent. A “daily dose” can be a single tablet or capsule or several tablets or capsules to be taken on a given day. Also, a daily dose of the compound, a prodrug thereof, or a pharmaceutically acceptable salt of the compound can consist of one tablet or capsule while a daily dose of the second compound can consist of several tablets or capsules and vice versa. The memory aid should reflect this.

It will be readily apparent to one of ordinary skill in the relevant art that other suitable modifications and adaptations to the methods and applications described herein can be made without departing from the scope of the invention or any embodiment thereof.

The following examples are offered to illustrate but not to limit the invention.

### EXPERIMENTAL

It is contemplated that a variety of compounds will exhibit an effect for the treatment of acute coronary syndrome. For example, in Table A provided above, compounds 2, 3, 15, 33, 57, 67, 84, 92, 96, 141, 154, 162, or 169 generally exhibit p38 MAP kinase activity as observed in an assay similar to the phosphorylation assay described above (see Kumar).

### Example 1

Cryopreserved human peripheral blood monocytes (HPBMCs, from Biowitker, Walkersvill, Md.) were thawed and washed twice in RPMI 1640. Cells were plated in tissue culture plates and incubated in 5% CO2 at 37 C. After 1 hr media were removed and cells were treated with various treatments. After 2 to 20 hours conditioned media and/or cell lysates were collected for the analyses of secreted cytokines, protein or mRNA expression. 10 μg/ml of purified human CRP (Calbiochem, San Diego, Calif.) was used in most of the treatments. In some experiments fetal calf serum (FCS from ATCC) was added to a final concentration of 10% together with CRP. In the examples provided herein, including those assessing the effects of p38 antagonism, cells were pre-treated with SD, a p38 small molecule inhibitor (Compound 162 in Table A) for 1 hour prior to CRP treatment as described above. In a parallel series of experiments, the effect of vehicle (DMSO) was tested. For TaqMan analyses of various gene expression cells were lysed in ice-cold RLT lysis buffer (Qiagen, Valencia, Calif.) and the lysates were kept in −80° C. until the analyses. For Western analyses cells were lysed in RIPA buffer (50 mM Tris-HCl, pH 7.5, 150 mM NaCl, 1 mM EDTA, 1% NP-40) containing phosphatase inhibitor cocktails and protease inhibitor cocktail (Promega, Madison, Wis.).

P38 MAP Kinase Activation

Level of activated p38 MAP kinase was determined by Western analysis of phosphorylated p38 MAP kinase. HPBMCs (1.5×10⁶ cell suspension in RPMI 1640) were plated in each 48-well tissue culture well. After 1 hr media were replaced with RPMI 1640 medium, medium containing 10 μg/ml of purified human CRP, 10% FCS or 10 μg/ml of purified human CRP plus 10% FCS. After 5 minutes media were removed from the wells and cells lysed in 100 μl of RIPA buffer. Insoluble materials were precipitated by centrifugation at 14,000 rpm for 10 minute in microfuge and supernatants were saved for Western analyses. Protein concentrations of the lysates were determined by BCA assay (Promega, Madison, Wis.). 10 μg total proteins were resolved on LDS-PAGE (In Vitrogen, Carlsbad, Calif.), and Western analyses for phospho-p38 MAP kinase was performed employing antibodies directed to diphospho-p38 (Biosource International, Camarillo, Calif.). For normalization, GAPDH levels were used. GAPDH level was determined by Western using antibody toward GAPDH (Biogenesis, Kingston, N.H.). Goat anti-rabbit IgG-HRP and goat anti-mouse IgG-HRP were from Cell Signaling (Beverly, Mass.).

Cytokine ELISA.

Conditioned media from the treated cells were collected (6 to 20 hrs), and the level of secreted interleukin 6 (IL-6), interleukin 1 beta (IL-1 β), tumor necrosis factor alpha (TNFα), and interleukin 8 (IL-8) was determined by ELISA according to the manufacturer's instruction (Biosource International, Camarillo, Calif.). All determinations were performed in duplicates.

Real-time PCR

RNA was extracted from the cells using Qiagen RNA extraction kit according to the manufacturer's instruction. The expression of pro-inflammatory genes IL-1β, IL-6, IL-8, TNFα, COX-2, ET-1, TF were assessed by real-time PCR. All determinations were performed in triplicates.

Real-time RT-PCR was performed in a two-step manner. cDNA synthesis and real-time detection were carried out in a PTC-100™ Thermal Cycler (MJ Research Inc, Waltham, Mass.) and an ABI Prism™ 7700 Sequence Detection System (Applied Biosystems, Foster City, Calif.), respectively. Random hexamers (Qiagen, Valencia, Calif.) were used to generate cDNA from 200 ng RNA as described in Applied Biosystems User Bulletin #2. TaqMan™ PCR Core Reagent Kit or TaqMan™ Universal PCR Master Mix (Applied Biosystems) were used in subsequent PCR reactions according to the manufacturer's protocols. Relative quantitation of gene expression was performed using the relative standard curve method.

Sequence specific primers and probes were designed using Primer Express Version 2 software (Applied Biosystems). Sequences of primers and probes can be found in Table 1. Expression levels were normalized to 18S rRNA.

TABLE 1
Sequence of oligonucleotide primers and probes used for real-time RT-PCR

| | | | | |
|---|---|---|---|---|
| Gene | Acession | Forward | Probe | Reverse |

| | | | | |
|---|---|---|---|---|
| 18S | M10098 | GCCGCTAGAGGTGAAATTCTTG | ACCGGCGCAAGACGGACCAG | CATTCTTGGCAAATGCTTTCG |
| COX2 | NM_000963 | GCTCAAACATGATGTTTGCATTC | TTGCCCAGCACTTCACGCATCAG | GCCCTCGCTTATGATCTGTCTT |
| Endothelin I | NM_001955 | TGCCACCTGGACATCATTTG | ACACTCCCGAGCACGTTGTTCCG | GACCTAGGGCTTCCAAGTCCAT |
| IL 1β | NM_000576 | TCAGCCAGGACAGTCAGCTCT | CCTTTCAGGGCCAATCCCCAGC | AGAGGCCTGGCTCAACAAAAG |
| IL6 | NM_000600 | ATGTAGCATGGGCACCTCAGAT | TGGTCAGAAACCTGTCCACTGGGCA | TAACGCTCATACTTTTAGTTCTCCATAGA |
| IL8 | NM_000584 | GCTCTCTTGGCAGCCTTCCT | ACCTTCACACAGAGCTGCAGAAA | TTAGCACTCCTTGGCAAAACTG |
| Tissue Factor | NM_001993 | CACCGACGAGATTGTGAAGGA | TGAAGCAGACGTACTTGGCACGGGT | AGAACCGGTGCTCTCCACAT |
| TNFα | NM_000594 | CCTCATCTACTCCCAGGTCCTCTT | CACCCATGTGCTCCTCACCCACAC | CTCTTGATGGCAGAGAGGAGGTT |

Tissue Factor Western

HPBMCs were treated as described above (p38 MAP kinase activation). After 16 hours media were removed and cells lysed as described. 10 μg total proteins were resolved on LDS-PAGE (In Vitrogen, Carlsbad, Calif.), and tissue factor protein expression was determined by Western using antibody against human tissue factor (American Diagnostica, Stamford, Conn.). Signals were normalized by GAPDH Western signal.

Results

CRP Activates p38 MAP Kinase.

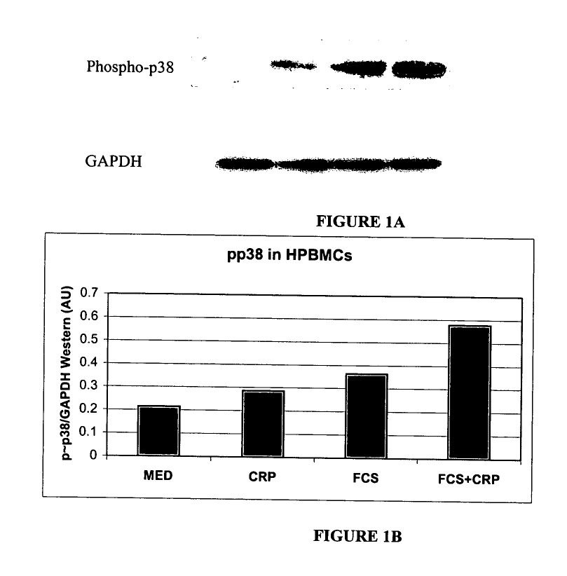

CRP was found to directly activate the p38 MAP kinase. As demonstrated in FIG. 1, at 5 minutes, CRP augmented the phosphorylation of p38 MAP kinase compared to control. FCS also increased p38 phosphorylation. Co-treatment of HPBMCs with FCS and CRP showed further phosphorylation of p38 above either CRP or FCS alone.

CRP Promotes Cytokine IL-6 Secretion via p38 Signaling.

IL-6 is the major cytokine that stimulates hepatic production of CRP upon acute inflammation setting. CRP in turn has been reported to induce IL-6 in monocytes and endothelial cells. Increased production of IL-6 represents a positive feedback mechanism for the production of CRP from the liver. IL-6 has been implicated in the pathogenesis and clinical course of atherosclerotic vascular disease.

IL-6 increases adhesion molecules and chemokines by the endothelium, increases hepatic release of fibrogen and has procoagulant effects on platelets. IL-6, by binding to sIL-6R, exerts effects in cells that lack the IL-6R per se and potentiate its own biological activity. The IL-6/sIL-6R complex stimulates leukocyte recruitment and promotes endothelial cell inflammatory responses. CRP is a physiological regulator of sIL-6R shedding in human neutrophils and markedly increases the formation of the sIL-6R/IL-6 complex.

IL-6 has been reported to mediate the CRP augmented macrophage uptake of LDL.

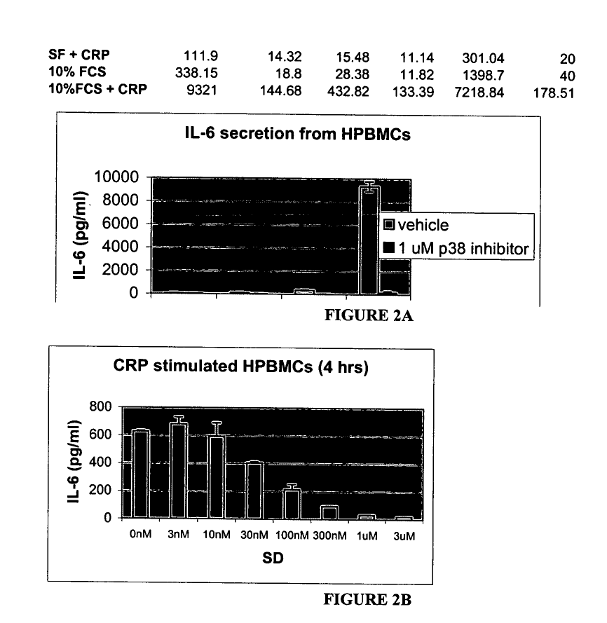

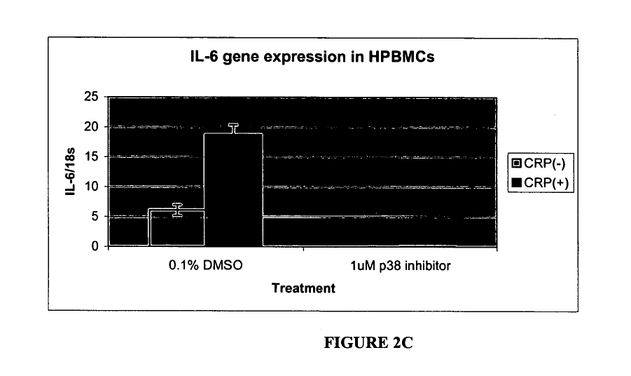

As part of the studies described herein, an investigation was done as to whether the CRP invoked IL-6 secretion from HPBMCs is dependent on p38 MAP kinase signaling. This was accomplished through the use of the p38 selective small molecule inhibitor SD. The effect of addition of 10% fetal calf serum (FCS) during the CRP treatment on IL-6 secretion was also assessed. As shown in FIG. 2a CRP alone, in the absence of FCS, slightly augmented the secretion of IL-6 from HPBMCs at 6 hours compared to that of control. FCS alone also induced CRP secretion moderately from HPBMCs. When the HPBMCs were incubated with CRP together with 10% FCS, the secretion of IL-6 was dramatically augmented. The level of IL-6 from the co-treatment of HPBMCs with CRP and FCS was greater than added levels of IL-6 induced by CRP and FCS, showing the synergy of FCS and CRP on IL-6 secretion in HPBMCs. CRP invoked IL-6 secretion appears to be very sensitive to p38 antagonism. As shown in FIG. 2a treatment of HPBMCs with 1 PM SD almost completely attenuated both basal and the CRP-induced IL-6 secretion. Similar inhibition was shown on FCS induced IL-6. IL-6 secretion induced by CRP plus 10% FCS was also dramatically inhibited with 1 μM SD. FIG. 2b shows the dose dependent inhibition of IL-6 secretion invoked by CRP in the presence of FCS. Less than 50 nM of SD was effective to inhibit 50% of IL-6 secretion and 3 μM SD almost completely inhibited the IL-6 secretion. As the strong activity of CRP is shown in the presence of FCS further experiments of CRP treatment were carried out in the presence of 10% FCS. Transcriptional effect of CRP on IL-6 gene expression was examined by real-time PCR. As shown in FIG. 2c, CRP increased the IL-6 mRNA level over FCS alone at 6 hours. Both FCS and CRP invoked IL-6 mRNA expression were almost completely blocked by 1 μM SD. These results suggest that CRP induced IL-6 secretion is mediated by p38 MAP kinase signaling, and p38 MAP kinase seems to regulate the transcription of IL-6 mRNA.

CRP Promotes Chemokine IL-8 Secretion via p38 Signaling.

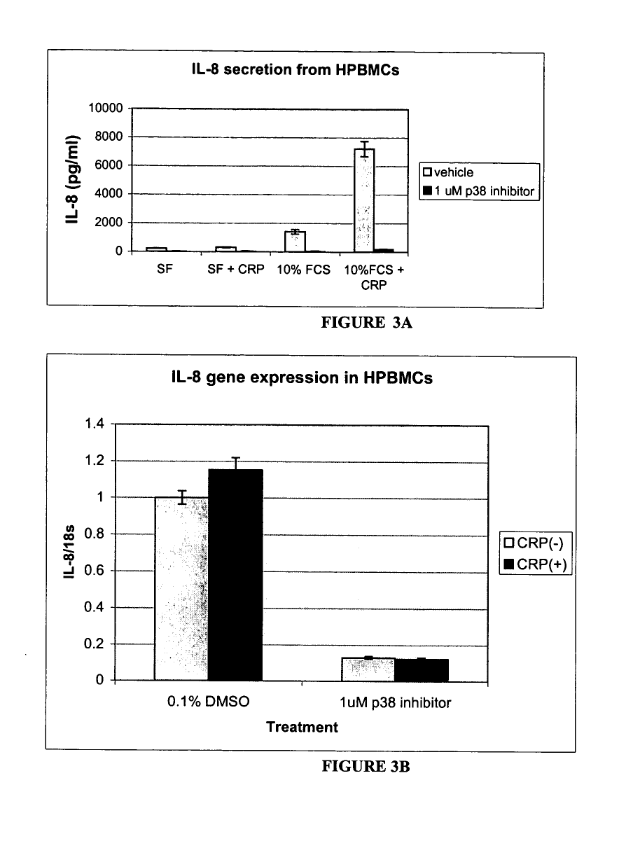

Elevated IL-8 has been reported in macrophage rich plaque area of atheroma. This chemokine may recruit further leukocytes to the plaques area, amplifying the local inflammation in affected area. IL-8 also has been shown to promote firm adhesion of monocytes to endothelial cells. CRP exerts similar effects on IL-8 secretion in HPBMCs as on IL-6. As shown in FIG. 3a CRP alone moderately augmented IL-8 secretion but strongly augmented IL-8 secretion when co-treated with FCS, showing synergistic effects with added FCS. When pre-treated with 1 μM SD IL-8 secretion was almost completely inhibited from HPBMCs in all the conditions. These results suggest that IL-8 secretion invoked by CRP is dependent on p38 signaling. Similar to IL-6, CRP increases the IL-8 mRNA level at 6 hours. This effect was almost completely inhibited by 1 μM SD (FIG. 3b). FCS invoked IL-8 expression was also almost completely inhibited by 1 μM SD.

### Example 2

CRP Induces TNFα and IL-1β Secretion via p38 Pathway.

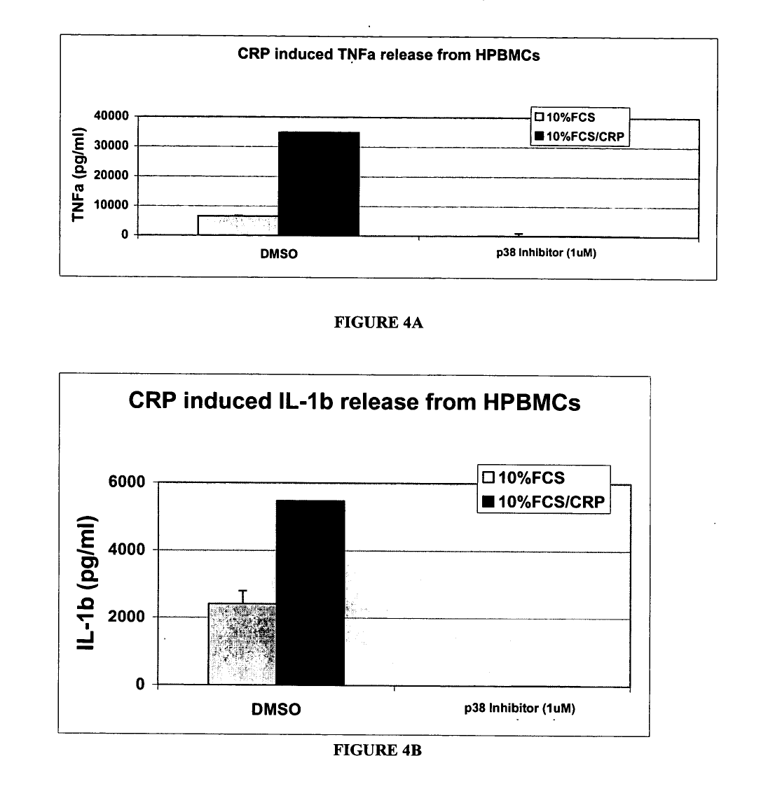

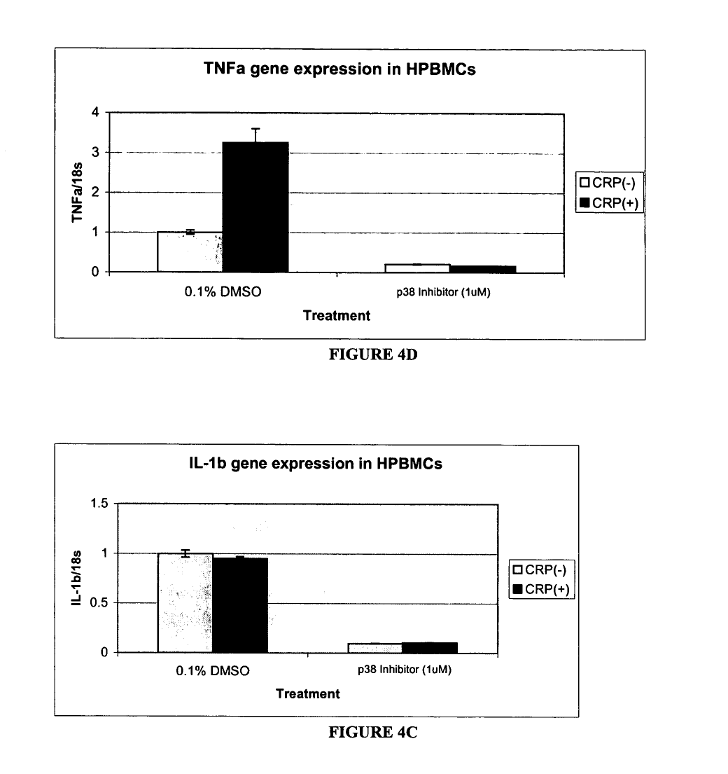

TNFα and IL-β are initial inflammatory cytokines produced when monocytes are activated during infalmmation. These cytokines activate variety of cells to produce further pro-inflammatory mediators and growth factors, amplifying the inflammation cascade. TNFα and IL-1 β are able to regulate the hepatic synthesis of CRP and induce synthesis and secretion of IL-6. These cytokines are implicated in the inflammatory process in atherosclerotic plaque and in acute coronary syndromes. Elevated CRP, TNFα and IL-1β have been reported in plaque area of atheroma. CRP has been reported to induce these cytokines in vitro. As pert of the studies referenced herein, an investigation as to whether CRP induced TNFα and IL-1β in HPBMCs are regulated via p38 MAP kinase signaling. HPBMCs were incubated with either 10% FCS or CRP plus 10% FCS for 6 hours and secreted cytokines TNFα and IL-1β were measured by ELISA. For p38 antagonism HPBMCs were treated with 1 μM SD prior to CRP treatment. FIG. 4a shows that CRP, in the presence of FCS, augments the secretion of TNFα in HPBMCs compared to that induced by FCS alone at 6 hours. Treatment with 1 μM SD strongly inhibited CRP-invoked TNFα secretion, suggesting that TNFα secretion induced by CRP is dependent on p38 signaling. FCS induced TNFα was also sensitive to p38 inhibition. At transcriptional level CRP, at 2 hours, increased the TNFα mRNA level (FIG. 4b). This CRP augmented TNFα transcription was completely attenuated with 1 μM SD. FCS induced TNFα transcription was also sensitive to SD treatment. Similar to TNFα secretion, CRP treatment augmented IL-1β secretion over FCS treatment alone at 6 hours (FIG. 4c). However, unlike CRP induced TNFα transcription, transcription of IL-1β was not augmented by CRP treatment above the level induced by 10% FCS at 2 hrs (FIG. 4d). FCS induced expression of IL-1β gene was attenuated by 1 μM SD. These results suggest that CRP induced TNFa secretion is regulated by p38 MAP kinase at transcriptional level, however, CRP induced IL-1β secretion may be regulated by p38 MAP kinase post-transcriptionally.

### Example 3

CRP Augments COX-2 Gene Expression via p38 Signaling.

COX-2 is important inflammatory mediator. COX-2 mRNA levels are reported to be higher in plaque tissue than in normal artery. Selective COX-2 inhibition seems to improve endothelium-dependent vasodilation and reduces low-grade chronic inflammation and oxidative stress in coronary artery disease. Potential benefits of selective COX-2 inhibitor in patients with acute coronary syndromes have been reported.

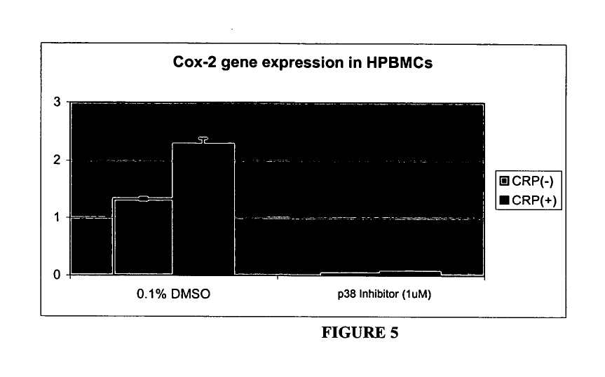

To investigate whether CRP promotes COX-2 expression in monocytes and the expression is mediated via p38 MAP kinase signaling HPBMCs were incubated either with 10% FCS or CRP plus 10% FCS in the presence or absence of SD, and the COX-2 mRNA level assessed by real-time PCR. At 6 hours, addition of CRP augmented COX-2 gene expression in HPBMCs (FIG. 5). Pre-treatment of HPBMCs with 1 μM SD almost completely blocked the CRP-invoked COX-2 mRNA expression. FCS induced COX-2 mRNA was also completely inhibited by 1 μM SD.

### Example 4

CRP Augments Potent Vasoactive Gene Endothelin-1 Expression via p38 Signaling.

ET-1 is one of the most potent endogenous vasoconstrictors and mediates a host of responses, including endothelial dysfunction, vasomotor contraction, leukocyte and platelet activation, and cellular proliferation. Additionally, it augments the vascular actions of other vasoactive substances, such as A-II, norepinephrine, and serotonin.

In endothelial cells, CRP is reported to partially exert its inflammatory function via endothelin, which can induce IL-6 secretion, amplifying the inflammatory cascade. Antagonism of ET-1 and IL-6 attenuates the expression of vascular cell adhesion moecule (VCAM-1), intracellular adhesion molecule (ICAM-1) and MCP-1. ET-1 has been reported to mediate the CRP augmented macrophage uptake of LDL.

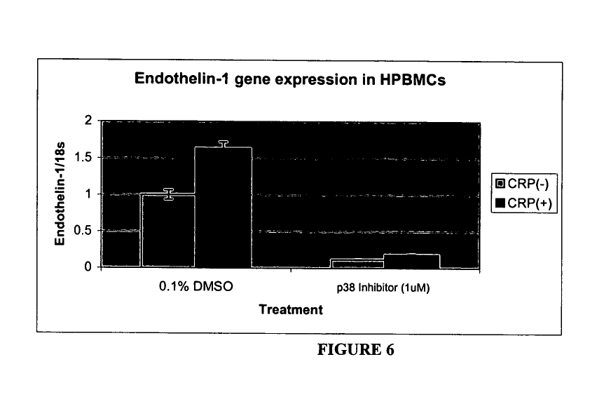

To investigate whether CRP can induce endothelin-1 in monocytes and whether it is mediated via p38 MAP kinase signaling HPBMCs were treated with CRP in the presence or absence of 1 μM SD, and ET-1 mRNA level was determine by real-time PCR. As shown in FIG. 6, addition of CRP augmented ET-1 gene expression moderately (1.7 fold) in HPBMCs at 6 hours. Pre-treatment of HPBMCs with 1 μM SD almost completely blocked the CRP-invoked ET-1 mRNA expression. FCS induced ET-1 mRNA was also completely inhibited by 1 μM SD

### Example 5

CRP Augments Prothrombotic Tissue Factor Expression via p38 Signaling.

The acute inflammatory response is frequently accompanied by serious thrombotic events. Tissue factor activates extrinsic coagulation pathway and thus may lead to thrombosis, vascular occlusion and myocardial infarction. Tissue factor level is increased in plasma from patients with acute coronary syndrome and TF protein is found in atheromatous plaques. In patients with disseminate intravasular coagulation TF mRNA in leukocytes is significantly higher in patients with higher CRP. In vitro CRP induces procoagulant activity via tissue factor expression in monocytes.

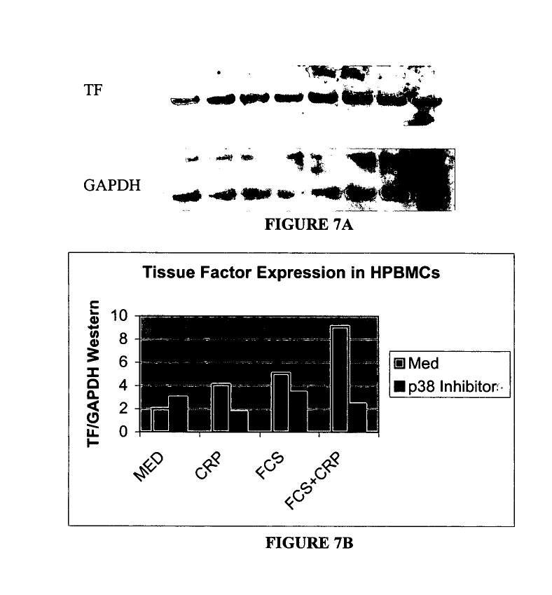

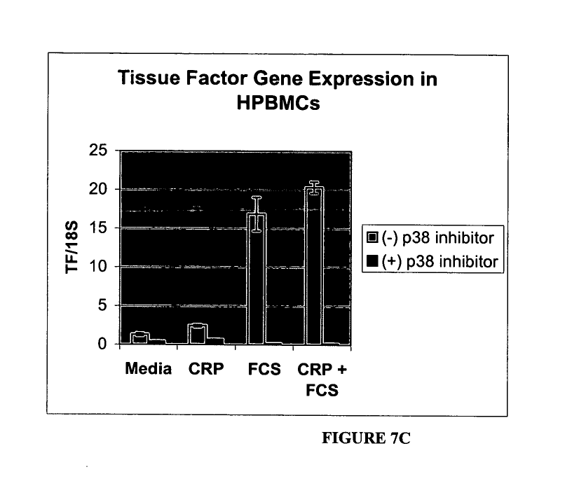

An evaluation was done as to whether this CRP invoked tissue factor expression is mediated via p38 MAP kinase signaling in HPBMCs. As shown in FIGS. 7a and 7(b), CRP alone increased the expression of TF protein moderately. 10% FCS also augments the TF protein expression. CRP plus FCS further augmented TF protein level. The increase in TF protein shows very similar pattern shown in phospho-p38 level with the same treatments. Treatment with 1 μM SD reduced TF protein expression invoked by CRP, FCS and CRP plus FCS to control level. TF expression is regulated by p38 MAP kinase at mRNA level. As shown in FIG. 7c CRP moderately increased TF mRNA expression in the presence or absence of FCS. Both basal and CRP induced TF mRNA expression were almost completely inhibitied with 1 μM SD.

Results

The results from the examples above suggest that CRP induces pro-inflammatory and prothrombotic responses in HPBMCs via p38 signaling, and p38 regulates these mediator expressions at transcriptional and/or post-transcriptional level. They demonstrate that CRP directly activates p38 MAP kinase, promotes the production and expression of several pro-inflammatory mediators that are important in coronary vascular disease, and the process is mediated by p38 MAP kinase signaling. CRP-invoked activation of p38 MAP kinase in HPBMCs was rapid. Activation of p38 MAP kinase has been closely linked to variety of inflammatory cascade in monocytes. Also demonstrated is that CRP directly facilitated the production of cytokines IL-6, IL-1β, and TNFα and chemokine IL-8 from HPBMCs via p38 MAP kinase signaling. CRP showed great synergy with FCS in the production of IL-6 and IL-8. Additional activation of p38 MAP kinase by CRP in the presence of FCS cannot explain dramatic increase in the level of IL-6 and IL-8 production. This suggests that CRP not only simply activates p38 MAP kinase but also amplifies inflammation by promoting synergy with other unknown factors. Synergy between CRP and other factors has been shown in various settings of inflammation.

In monocytes CRP showed a synergistic effect on LPS-dependent IL-1β production. CRP also acts synergistically with lipopolysaccharide and interferon γ to induce tissue factor production by monocytes. CRP also acts synergistically with lipopolysaccharide in the activation of endothelial cells.

In the clinical setting enhanced response of blood monocytes, when challenged with LPS in vitro, was observed in patients with recurrent unstable angina with elevated CRP. This suggests that CRP may amplify the intensity of the inflammatory response by further inducing proinflammatory mediators and working synergistically with other mediators.

As shown herein, CRP augmented potent prothrombotic protein TF expression in HPBMCs and TF protein and mRNA expressions were blocked by SD. CRP augmented other inflammatory genes COX-2 and ET-1 expression in HPBMCs. Both of them are known to promote inflammation and vasoconstriction. Constriction of the vessel may render the vulnerable plaque to rupture. These gene expressions were almost completely inhibited by SD. CRP, by increasing TF expression and increasing the risk of plaque rupture via TNFα, IL-1β, IL-6, Cox-2 and ET-1, can greatly increase the risk of thrombosis that can be catastrophic. Increased thrombosis after arterial injury has been observed in transgenic mice that express human CRP (Danenberg, 2003). P38 inhibition seems to control the underlying inflammation invoked by CRP at multiple levels.

CRP directly facilitates expressions of cytokines IL-6, IL-8, IL-1β and TNFα, and COX-2 and vasoactive gene ET-1 and prothrombotic gene TF in HPBMCs. These effects seem to occur via p38 signaling and are attenuated during pharmacological intervention with SD. The data presented herein is a novel demonstration that CRP induced inflammation in HPBMC cells involve p38 MAP kinase signaling. Understanding the mechanisms and mediators of the pro-inflammatory activities of CRP suggest a novel therapeutic strategy. p38 inhibition offers such an opportunity for the treatment of cardiovascular disease.

What is claimed is:

1. A method of treating or preventing vascular endothelial disease in a patient in need of such treatment, said method comprising administering a pharmaceutically effective amount of a p38 inhibitor to said patient.

2. A method of treating or preventing atherosclerosis in a patient in need of such treatment, said method comprising administering a pharmaceutically effective amount of a p38 inhibitor to said patient.

3. A method of treating or preventing arterial inflammation in a patient in need of such treatment, said method comprising administering a pharmaceutically effective amount of a p38 inhibitor to said patient.

4. A method to treat or prevent acute coronary syndrome in a patient in need of such treatment, said method comprising administering a pharmaceutically effective amount of a p38 inhibitor to said patient

5. The method of claim 2 wherein said method results in the stabilization of plaque associated with atherosclerosis.

6. A method for treating or preventing ischmemia associated with atherosclerosis in a patient in need thereof, said method comprising administering a pharmaceutically effective amount of a p38 inhibitor to said patient.


SEQUENCE LISTING (24 sequences)

SEQ ID NO: 1 - DNA, 22 bp, Homo sapiens
gccgctagaggtgaaattcttg

SEQ ID NO: 2 - DNA, 20 bp, Homo sapiens
accggcgcaagacggaccag

SEQ ID NO: 3 - DNA, 21 bp, Homo sapiens
cattcttggcaaatgctttcg

SEQ ID NO: 4 - DNA, 24 bp, Homo sapiens
cgctcaaacatgatgtttgcattc

SEQ ID NO: 5 - DNA, 23 bp, Homo sapiens
ttgcccagcacttcacgcatcag

SEQ ID NO: 6 - DNA, 22 bp, Homo sapiens
gccctcgcttatgatctgtctt

SEQ ID NO: 7 - DNA, 20 bp, Homo sapiens
tgccacctggacatcatttg

SEQ ID NO: 8 - DNA, 23 bp, Homo sapiens
acactcccgagcacgttgttccg

SEQ ID NO: 9 - DNA, 22 bp, Homo sapiens
gacctagggcttccaagtccat

SEQ ID NO: 10 - DNA, 21 bp, Homo sapiens
tcagccaggacagtcagctct

SEQ ID NO: 11 - DNA, 22 bp, Homo sapiens
cctttcagggccaatccccagc

SEQ ID NO: 12 - DNA, 21 bp, Homo sapiens
agaggcctggctcaacaaaag

SEQ ID NO: 13 - DNA, 22 bp, Homo sapiens
atgtagcatgggcacctcagat

SEQ ID NO: 14 - DNA, 25 bp, Homo sapiens
tggtcagaaacctgtccactgggca

SEQ ID NO: 15 - DNA, 29 bp, Homo sapiens
taacgctcatacttttagttctccataga

SEQ ID NO: 16 - DNA, 20 bp, Homo sapiens
gctctcttggcagccttcct

SEQ ID NO: 17 - DNA, 23 bp, Homo sapiens
accttcacacagagctgcagaaa

SEQ ID NO: 18 - DNA, 22 bp, Homo sapiens
ttagcactccttggcaaaactg

SEQ ID NO: 19 - DNA, 21 bp, Homo sapiens
caccgacgagattgtgaagga

SEQ ID NO: 20 - DNA, 25 bp, Homo sapiens
tgaagcagacgtacttggcacgggt

SEQ ID NO: 21 - DNA, 20 bp, Homo sapiens
agaaccggtgctctccacat

SEQ ID NO: 22 - DNA, 24 bp, Homo sapiens
cctcatctactcccaggtcctctt

SEQ ID NO: 23 - DNA, 24 bp, Homo sapiens
cacccatgtgctcctcacccacac

SEQ ID NO: 24 - DNA, 23 bp, Homo sapiens
ctcttgatggcagagaggaggtt